SEQUENCE LISTING (10 sequences)

SEQ ID NO: 1 - protein, 8 aa, Artificial Sequence
  Synthetic
SIINFEKL

SEQ ID NO: 2 - protein, 13 aa, Mus musculus
CMRYGNYWYFDVW

SEQ ID NO: 3 - protein, 14 aa, Mus musculus
CMRYGSSYWYFDVW

SEQ ID NO: 4 - protein, 13 aa, Mus musculus
CMRYSNYWYFDVW

SEQ ID NO: 5 - protein, 13 aa, Mus musculus
CMRYGNYWYFDVW

SEQ ID NO: 6 - protein, 8 aa, Mus musculus
CARSFAYW

SEQ ID NO: 7 - protein, 12 aa, Mus musculus
CARGLLYAMDYW

SEQ ID NO: 8 - protein, 10 aa, Mus musculus
CARLYYFDYW

SEQ ID NO: 9 - protein, 11 aa, Mus musculus
CARRDYGFAYW

SEQ ID NO: 10 - protein, 7 aa, Mus musculus
CARFAYW

(19) United States
(12) Patent Application Publication
(10) Pub. No.: US 2024/0209313 A1
(43) Pub. Date: Jun. 27, 2024

(54) B-CELL BASED IMMUNOTHERAPY FOR THE TREATMENT OF GLIOBLASTOMA AND OTHER CANCERS

(71) Applicant: Northwestern University, Evanston, IL (US)

(72) Inventors: Catalina Lee-Chang, Skokie, IL (US); Maciej S. Lesniak, Evanston, IL (US)

(73) Assignee: Northwestern University, Evanston, IL (US)

(21) Appl. No.: 18/021,687

(22) PCT Filed: Aug. 17, 2021
(86) PCT No.: PCT/US21/46331

§ 371 Date: Feb. 16, 2023

Related U.S. Application Data
(60) Provisional application No. 63/066,533, filed on Aug. 17, 2020.

(51) Int. Cl. C12N 5/0781; A61P 35/00

(52) U.S. Cl. CPC C12N 5/0635; A61P 35/00; C12N 2501/24

(57) ABSTRACT

The present invention provides methods of making anti-cancer compositions by activating 4-1BBL+ B cells using a CD40 agonist and IFNγ. The anti-cancer compositions produced by these methods may be used as an immunotherapy for the treatment of cancer. either alone or in combination with radiation, chemotherapeutics and/or checkpoint blockade.

### CROSS-REFERENCE TO RELATED PATENT APPLICATIONS

This application claims the benefit of priority under 35 U.S.C. § 119(e) to U.S. Provisional Application No. 63/066,533 filed Aug. 17, 2020, the content of which is incorporated herein by reference in its entirety.

### STATEMENT REGARDING FEDERALLY SPONSORED RESEARCH

This invention was made with government support under Grant Numbers P50 CA221747, R35 CA197725, and R01 NS093903 awarded by the National Institutes of Health. The government has certain rights in this invention.

### SEQUENCE LISTING

A Sequence Listing accompanies this application and is submitted as an ASCII text file of the sequence listing named “702581_02007_ST25.txt” which is 2009 bytes in size and was created on Aug. 17, 2021. The sequence listing is electronically submitted via EFS-Web with the application and is incorporated herein by reference in its entirety.

### BACKGROUND

Despite the tremendous effort in basic, translational, and clinical research, the standard-of-care of patients with glioblastoma (GBM) has been virtually unchanged for the past two decades (Stupp et al., 2017), aside from tumor treating fields (Taphoorn et al., 2018). GBM is one of the immunologically “coldest tumors” where T-cell exclusion is at its maximum, and myeloid infiltration predominates (Thorsson et al., 2018). This is due to profound immunosuppression (Raychaudhuri et al., 2011; Wintterle et al., 2003), the metabolically hostile microenvironment (Li et al., 2009), and the low mutational burden of these tumors (Iranzo et al., 2018). Together, these barriers have hindered the development of effective immunotherapies (Vega et al., 2008; Wainwright et al., 2012b).

The first immunotherapies tested in the clinic used dendritic cell (DC)-based vaccines as a way to promote endogenous immunity against GBM (Eagles et al., 2018; Prins et al., 2013; Wen et al., 2019). In many of these trials, DCs were pulsed with autologous tumor lysate, while others injected immunogenic epitopes against tumor-associated antigens (Weller et al., 2017). To date, these approaches have only met with limited success, although many more trials are still underway (Eagles et al., 2018). Several emerging therapeutic strategies, such as checkpoint blockade (Maxwell et al., 2017) or adoptive transfer of chimeric antigen-receptor (CAR) T cells targeting GBM-antigens, are also being explored (Pituch et al., 2018). However, their effectiveness remains to be determined. While immunotherapy remains an attractive approach for GBM patients, immunotherapeutic approaches have yet to significantly promote anti-tumor immunity in these patients to achieve a clinical benefit.

The B-cell-based vaccine is a promising yet under-investigated approach to boost anti-cancer immunity (Kim et al., 2014; Schultze et al., 1997). There are three main advantages of B cells as cellular-based vaccines: i) they can be readily manufactured ex-vivo; ii) they can share cognate antigen (Ag)-specificity with T cells (Wennhold et al., 2017); iii) they have high mobility, which allows their homing to key secondary lymphoid organs as well as tumor (Gonzalez et al., 2015).

However, a key reason why B-cell anti-tumor vaccines have not garnered more interest is that B cells can quickly switch between anti- to pro-tumorigenic phenotype within the surrounding microenvironment. For example, B cells become immunosuppressive within GBM and represent about 10% of infiltrating immune cells (Lee-Chang et al., 2019). Yet tumor-infiltrating B cells do show an anti-tumor effect in a variety of cancers (Tsou et al., 2016). Their function has been linked to the production of anti-tumor antibodies (Garaud et al., 2019), and their antigen-presenting cell (APC) function and activation of effector T cells (Bruno et al., 2017; Nielsen et al., 2012). Thus, there remains an unmet need in the art for B-cell anti-tumor vaccines that are resistant to tumor immunosuppression.

### SUMMARY

Provided herein are methods of making an anti-cancer composition by (a) collecting 4-1BBL+(CD137L+) B cells; (b) incubating the B cells with a CD40 agonist; (c) adding IFN-γ to the B cells; and (d) contacting the B cells with tumor-derived antigens.

Also provided are compositions including the B cells made by the method. The compositions include 4-1BBL+B cells activated in vitro with a CD40 agonist to increase expression of CD86 and IFN-γ receptor and further incubated with IFN-γ for at least 20 hours and finally pulsed with tumor-derived antigen, such that the B cells are effective tumor-derived antigen presenting cells.

In a further aspect, the invention provides methods of using the B cell compositions to treat a subject with cancer by administering an effective amount of the composition to the subject.

### BRIEF DESCRIPTION OF THE DRAWINGS

FIG. 1 demonstrates that 4-1BBL⁺ B cells in GBM patients' peripheral blood have antigen-presenting function. (A) Box-plot showing the expression of 4-1BBL in CD19⁺CD20⁺B cells from newly diagnosed GBM patients' PBMC (n=90). (B) Histograms representing the intracellular expression of TNFα and IFNγ, and surface expression of CD69 and CD86 by 4-1BBL⁻ (black line) and 4-1BBL⁺ (blue line) B cells. (C) Linear regression analysis of 4-1BBL expression by B cells and CD69 expression by CD8⁺ T cells in newly diagnosed GBM patient's PBMC (n=68). (D-E) CD8⁺ T-cell co-stimulation assay using CD8⁺ T cells activated with anti-CD3 and IL2 mixed with autologous 4-1BBL⁻ and 4-1BBL⁺ B cells from peripheral blood of 3 newly diagnosed GBM patients (NU00856, NU01006, and NU00429). CD8⁺ T-cell activation was measured as cellular expansion (D) and expression of intracellular IFNγ and GzmB (E). The experiment was performed in triplicate. (F) B_{Vax} from GL261-OVA mice were pulsed with SIINFEKL (SEQ ID NO: 1) (B_{Vax(SIINFEKL)}) and evaluated for the SIINFEKL (SEQ ID NO: 1) presentation by MHC class I (H-2K^{b+} SIINFEKL (SEQ ID NO: 1) antibody) and the co-expression of MHC class I (H-2K^{b}) and co-stimulatory molecules CD86 and 4-1BBL. Shown are representative experiments of 3 independent experiments. (G) B_{Vax} were tested for their ability to uptake AlexaFluor488-conjugated OVA [B_{Vax(OVA)}], and (H) present SIINFEKL (SEQ ID NO: 1) peptide via MHC class I (H-2K^{b}). Surface transport of the H-2K^{b}+SIINFEKL (SEQ ID NO:

1) complex was inhibited using Brefeldin A (BFA). Shown, a representative experiment of 3 independent experiments. (I) B_{Naive}, B_{Naive}⁺IFNγ, B_{Vax} and DCs were pulsed with OVA and tested for their ability to promote OT-I CD8⁺ T-cell activation assessed by cell proliferation (expansion index, X-axis) and intracellular expression of GzmB (Y-axis). The experiment was performed in triplicate. Shown, a representative experiment of 2 independent experiments. (J) OT-I CD8⁺ T cells cultured with B_{Naive}, B_{Vax} pulsed with OVA and isotype control, B_{Vax(OVA)}⁺ IC, or with MHC class I blocking Ab, B_{Vax(OVA)}⁺ anti-H2K_{b}, and tested for their cellular expansion. Shown, a representative experiment of 2 independent experiments. All histograms are shown as mean±SD. Statistical significance is depicted as ns: not statistically significant, *p<0.05, **p<0.01, ***p<0.001, ****p<0.0001.

FIG. 2 characterizes B_{Vax} antigen-presenting cell (APC) function in vivo. (A) Rag1 deficient mice were challenged intracranially with GL261 overexpressing Ovalbumin (GL261-OVA). Nine days after, mice received intravenously B_{Naive} or B_{Vax} pulsed with OVA protein. eFluor450-labeled CD8⁺ T cells from WT C57BL/6 mice were concomitantly injected with B cells (n=4 mice/group). Seven days after the cell adoptive transfer, eFluor450⁺ CD8⁺ T cells were evaluated by flow cytometry in the tumor-bearing brains and the deep cervical lymph nodes (dCLN). Shown, a representative experiment of 2 independent experiments. (B) B-cell deficient (BKO) mice were challenged intracranially with GL261-OVA. Nine days after mice received intravenously B_{Naive} or B_{Vax} pulsed with OVA protein. A group of B_{Vax(OVA)} was pretreated with pertussis toxin (PTX) before injection (n=4 mice/group). Seven days after the cell adoptive transfer, SIINFEKL (SEQ ID NO: 1) -specific CD8⁺ T cells were analyzed in the tumor-bearing brains by flow cytometry using SIINFEKL (SEQ ID NO: 1)-tetramer. Shown, a representative experiment of 2 independent experiments. (C) B-cell deficient (BKO) mice were challenged intracranially with CT2A cells. Nine days after mice received intravenously B_{Naive} and B_{Vax} pulsed with CT2A tumor lysates pretreated with or without PTX (n=5 mice/group). Seven days after the cell adoptive transfer, CD8⁺ T cells were evaluated for the intracellular expression of GzmB and IFNγ in the tumor-bearing brain, blood, and dCLN. Shown, a representative experiment of 3 independent experiments. (D) Rag1 deficient (KO) mice were challenged intracranially with CT2A cells. Nine days after, mice received intravenously and concomitantly both Cell Tracker® red CMPTX B_{Vax} (red) cells and CellTracker® green CMFDA-labeled CD8⁺ T cells (green). B_{Vax} and CD8⁺ T-cell splenic localization were analyzed by fluorescent microscopy. Bars represent 100 μm (left image, 20× magnification) and 50 μm (right image, 63× magnification). Images are representative of spleen and CLN of 3 mice. Histograms are shown as mean±SD. Statistical significance is depicted as ns: not statistically significant, *p<0.05, **p<0.01, ***p<0.001, ****p<0.0001.

FIG. 3 demonstrates that radiotherapy favors B-cell adaption in vivo. (A) CT2A-bearing mice were irradiated (RT) and CD45.1⁺ B_{Vax} were adoptively transferred intravenously. CD45.1⁺ cells were analyzed by flow cytometry in the spleen, dCLN and tumor-bearing brains (n=4 mice/group). (B) Irradiated CT2A-bearing mice were bled 36 hours and 5 days after (n=3 mice/group) after RT. Serum BAFF levels were analyzed and compared to non-irradiated (Tumor) and non-tumor-bearing mice serum (No tumor). (C) The same experiment as in (A) was performed using CD45.1⁺ B_{Naive}, B_{Act} (4-1BBL⁻ B cells activated with anti-CD40 and IFNγ) or B_{Vax}. CD45.1⁺ cells were analyzed by flow cytometry in the spleen and dCLN. B-cell proliferative status was assessed by the expression of Ki67. Depicted a representative animal for each group (n=3-4 mice/group). The experiment was repeated twice independently. (D) Alternatively, a group of mice received B cells pretreated with BAFF receptor blocking Ab. The treatment was also administered to the mice intravenously for 3 consecutive days after B-cell transfer. (E) Irradiated CT2A-bearing mice received intravenously with vehicle (Mock, black line), CD8⁺ T cells (gray line), pulsed B_{Vax} (black dashed line) or combined pulsed B_{Vax}+CD8⁺ T cells (pink dotted line). The experiment was performed using n=10 mice/group. Shown, a representative experiment of 3 independent experiments. (F) Irradiated CT2A-bearing mice received intravenously with vehicle (Mock, black line), CD8⁺ T cells (gray line), CD8⁺ T cells intravenously+pulsed DC_{Vax} administered either intradermally (DC_{Vax(id)}, dashed black line) or intravenously (DC_{Vax(iv)}, blue line), pulsed B_{Vax}+CD8⁺ T cells (pink dotted line). The experiment was performed using n=10 mice/group. Shown, a representative experiment of 2 independent experiments. (G) Irradiated CT2A-bearing mice received intravenously eFluor450-labeled B_{Vax} or DCs. DCs were administered either intravenously (DC₍ᵢᵥ₎) or intradermally (DC_{(id)}). Seven days after cell transfer eFluor450⁺ cells were analyzed in the dCLN and spleen (n=3-4 mice/group). Shown, a representative experiment of 2 independent experiments. Histograms are shown as mean±SD. Statistical significance is depicted as ns: not statistically significant, *p<0.05, **p<0.01, ***p<0.001, ****p<0.0001.

FIG. 4 demonstrates that B_{Vax} facilitate CD8⁺ T-cell tumor infiltration and proliferation. (A) CT2A-bearing mice were irradiated 7 days after tumor implantation. Forty-eight hours after mice received intravenously with DC or B_{Vax} together with CellTracker deep red-labeled CD8⁺ T cells. Far-red signal emitted by CD8⁺-cells was monitored at different time points (24, 30, 50 and 72 hours). The experiment was performed using 4 mice/group. One mouse did not receive any lymphocyte and was used as a blank. In all experiments, mice were randomized and were grouped by treatment for the sole purpose of image capture. (B) Alternatively, mice received DC or B_{Vax} (pulsed with CT2A lysates) concomitantly with CD8⁺ T cells from CD45.1 congenic mice. Forty-eight hours after, mice were evaluated for the proliferative status of adoptively transferred CD45.1⁺CD8⁺ T cells by measuring the expression of Ki67. This experiment was performed using 5 mice/group. (C) CT2A-bearing mice were irradiated 7 days after tumor injection. Twenty-four hours after irradiation, mice received intravenously with Cell Tracker® red CMPTX-labeled CD8⁺ T cells±B_{Vax} pulsed with CT2A protein lysates intravenously±anti-PD-L1 intraperitoneally. Seven days after, Cell Tracker® red CMPTX-labeled CD8⁺ T-cell persistence (% of CellTracker⁺ CD8⁺ T cells/total CD45⁺ leukocytes) was analyzed by flow cytometry in the tumor-bearing brains, the dCLN and superficial cervical lymph nodes (sCLN). (D) Adoptively transfer CD8⁺ T cells used in (C) were also phenotyped for CD44, CD62L, GzmB and IFNγ in the dCLN (after injection). The phenotype was compared to that before injection. All histograms are shown as mean±SD. Statistical significance is depicted as ns: not statistically significant, *p<0.05, **p<0.01, ***p<0.001, ****p<0.0001.

FIG. 5 demonstrates that B_{Vax} potentiate the therapeutic effect of combined RT+CD8⁺ T+PD-L1 blockade. (A) Irradiated CT2A-bearing mice received vehicle (Mock, black line), 3 injections of anti-PD-L1 (dotted black line), 3 injections of CD8⁺ T cells and anti-PD-L1 (gray line), or 3 injections of pulsed B_{Vax}+CD8⁺ T cells, and anti-PD-L1 (pink dotted line). A non-irradiated group was kept as control (No RT, dashed black line). Seventy-five days after tumor challenge (arrow), surviving mice were re-challenged with CT2A cells in the left hemisphere, opposite of the initial tumor injection site. (B) Long-term survivors (LTS) were sacrificed and checked for the presence of tumor mass using H&E staining. Non-tumor-bearing but skull drilled (no tumor) and age-matched CT2A-bearing mice (Control) were used as controls. Sections were performed as depicted in the cartoons. For LTS treated with B_{Vax} and CD8⁺ T cells (LTS-B_{Vax}+CD8), brains were sectioned both in the right hemisphere (1^{st} site of injection, LTS-B_{Vax}+CD8 R) and left hemisphere (2^{nd} site of injection—rechallenge, LTS-B_{Vax}+CD8 L). H&E sections images are representative of 3 LTS-B_{Vax}+CD8, 2 control and 1 no tumor brains. (C) The same brains utilized in (B) were used to stain for infiltrating CD8⁺ T cells. Representative images of one LTS-R section where the choroid plexus, site of injection, the pons (arrows) and the cerebellum (arrows) are magnified. Bars represent 100 μm. (D) Freshly dissected brains from no tumor (n=2), control (n=3), LTS that only received CD8⁺ T cells and PD-L1 blockade (LTS-CD8, n=4) and LTS-B_{Vax}+CD8 (n=5) mice were analyzed for lymphocytes phenotype. All histograms and flow cytometry data are shown as mean±SD. Statistical significance is depicted as ns: not statistically significant, *p<0.05, **p<0.01, ***p<0.001, ****p<0.0001.

FIG. 6 demonstrates that B_{Vax} enhanced animal survival in combination with GBM standard-of-care and PD-L1 blockade. (A) Mice received whole brain radiotherapy (B-RT) for 3 consecutive days (D7-D10, 3Gy each day) followed by 50mg/Kg of temozolomide (TMZ) for 5 consecutive days (D11-D16). Serum BAFF levels were measured by ELISA 36 hours after termination of the chosen therapy. (B) B_{Vax} from congenic CD45.1⁺ mice were adoptively transferred into CD45.2⁺ CT2A-bearing mouse (n=4 mice/group). Seven days after cell adoptive transfer, CD45.1⁺ cells were analyzed by flow cytometry in the spleen, dCLN and tumor-bearing brains (n=4 mice/group). (C) B_{Vax}+CD8⁺ T-cell therapeutic effect was tested in mice that received whole brain radiotherapy (B-RT). Non-irradiated (Mock, black line), irradiated (B-RT, grey line) were used as controls. Experimental groups received intravenously either CD8⁺ T cells (B-RT+CD8⁺ T cells, dashed black line), B_{Vax} (B-RT+B_{Vax}, dashed grey line) or both (B-RT+CD8⁺ T cells+B_{Vax}, dashed blue line). The experiment was performed using n=10 mice/group. (D) B_{Vax}+CD8⁺ T-cell therapeutic effect was tested in mice that treated with temozolomide (TMZ). Untreated (Mock, black line), and TMZ treated (TMZ, grey line) were used as controls. Experimental groups received intravenously either CD8⁺ T cells (TMZ+CD8⁺ T cells, dashed black line), B_{Vax} (TMZ+B_{Vax}, dashed grey line) or both (TMZ+CD8⁺ T cells+B_{Vax}, dashed blue line). The experiment was performed using n=10 mice/group. (E) B_{Vax}+CD8³⁰ T-cell therapeutic effect was tested in mice treated with B-RT and TMZ. Cellular therapy was administered 24 hours after TMZ treatment termination (D19 after tumor challenge). Untreated mice (Mock, black line), mice that only received 2 intravenous injections of B_{Vax}+CD8⁺ T cells (B_{Vax}+CD8⁺ T, grey line) and irradiated mice that received TMZ (B-RT+TMZ, dashed black line) were used as controls. Experimental groups treated with B-RT and TMZ received 2 intravenous injections of B_{Vax}+CD8⁺ T cells (B-RT+TMZ+B_{Vax}+CD8⁺ T, dashed grey line), 2 intraperitoneal injections of PD-L1 blockade (B-RT+TMZ+aPDL1, dashed blue line), 2 intravenous injections of CD8⁺ T cells and 2 intraperitoneal injections of PD-L1 blockade (B-RT+TMZ+CD8⁺ T+aPDL1, grey line), or 2 intravenous injections of B_{Vax}+CD8⁺ T cells and 2 intraperitoneal injections of PD-L1 blockade (B-RT+TMZ+B_{Vax}+CD8⁺ T+aPDL1, dashed pink line). The experiment was performed using n=10-11 mice/group. Histograms are shown as mean±SD. Statistical significance is depicted as ns: not statistically significant, *p<0.05, **p<0.01, ***p<0.001, ****p<0.0001.

FIG. 7 demonstrates that GBM patient-derived B_{Vax} promote anti-tumor CD8⁺ T cells. (A) Paired fresh peripheral blood and tumor were collected from newly diagnosed GBM patients (n=4). B_{Vax} were generated and pulsed with tumor lysates and co-cultured with autologous eFluor450-labeled CD8⁺ T cells. CD8⁺ T-cell activation was assessed by cell proliferation (eFluor450 fluorescence dilution measured as expansion index) and intracellular expression of GzmB. (B and C) Paired samples from primary GBM IDH WT (case NU 02120, B) and recurrent GBM IDH WT (NU02265, C). B_{Vax}-activated autologous CD8⁺ T cells were obtained as shown in (A) and tested for their ability to kill autologous glioma cells. Cell killing measurement were taken periodically for 12.5 hours using the IncuCyte S3 Live Cell Analysis System. Histograms are shown as mean±SD. Statistical significance is depicted as ns: not statistically significant, *p<0.05, **p<0.01, ***p<0.001, ****p<0.0001.

FIG. 8 demonstrates that B_{Vax} produce tumor-reactive antibodies with therapeutic effects. (A) CD45.1⁺ B_{Vax} were adoptively transferred into B-cell deficient CT2A-bearing mice. Seventy-two hours after, CD45.1⁺ B_{Vax} were evaluated for the expression of plasmablast marker CD138 by flow cytometry in the dCLN (n=4/group). (B) Schema of B_{Vax}-derived serum immunoglobulin (Ig) obtainment. (C) Diagram representing the distribution of different Ig subtypes from serum antibodies derived from B_{Naive}, B_{Act} and B_{Vax}. Ig subtype measurement of serum samples was performed by ELISA, and mean total Ig concentration is shown in the bottom of the diagram (mg/ml). (D) B-cell subsets IgG reactivity was measured by immunofluorescence (IF). Serum samples were incubated on tumor-bearing brains sections from B-cell deficient mice (BKO). Binding IgG was detected using anti-mouse IgG Cy5 (red) secondary antibody. Nuclei was detected using DAPI (blue), and myeloid cells were evaluated by using anti-mouse CD11b AF488 antibody (green). Bars represent 100 μm. Shown, a representative experiment of 4 different mice. (E) B_{Naive}, B_{Act} and B_{Vax} were generated from GL261 overexpressing ovalbumin (GL261-OVA) tumor-bearing mice. B cells were allowed to produce antibodies in GL261-OVA-bearing B-cell deficient mice (BKO). Serum samples were collected and IgG were purified and tested for their reactivity against OVA peptide SIINFEKL (SEQ ID NO: 1) by ELISA. Semi-quantitative measurement is shown and optical density (O.D). Serum from B-cell deficient mice and C57BL/6 SIINFEKL (SEQ ID NO: 1)-immunized mice were used as negative and positive control respectively (n=4/group). (F) Purified IgG were tested for their therapeutic effect in the CT2aA model. IgG were delivered intracranially for 3 consecutives days (12.5 μg/mouse/injection). Untreated mice (black line) were used as controls. Experimental groups received either B_{Naive}-derived IgG (B_{Naive} IgG, blue line) or B_{Vax}-derived IgG (B_{Vax} IgG, pink line). The experiment was performed using n=10 mice/group. Histograms are shown as mean±SD. Statistical significance is depicted as ns: not statistically significant, *p<0.05, **p<0.01, ***p<0.001, ****p<0.0001.

FIG. 9 shows a further characterization of 4-1BBL-expressing B cells. (A) 4-1BBL expression levels by CD19⁺ B cells in the brain, blood, deep cervical lymph nodes (dCLN) and superficial cervical lymph nodes (CLN) of CT2A glioma-bearing mice over the time: no tumor (DO), 7, 14, 21 days after tumor implantation (n-4 mice/time point). (B) Murine B-cell ability to promote CD8⁺ T-cell activation, measured by cell expansion and expression of intracellular granzyme B (GzmB) was assessed by activated B cells, and compared to B cells from 4-1BBL deficient (4-1BBL KO) mice. Representative experiment of a total of 3 independents experiments performed in triplicates. For all experiments shown in this figure, differences among multiple groups were evaluated using One-way ANOVA with post hoc Tukey's test and histograms are shown as mean±SD. In all experiments, statistical significance is depicted as ns: not statistically significant, *p<0.05, **p<0.01, ***p<0.001, ****p<0.0001.

FIG. 10 depicts B_{Vax} generation. (A) Human B cells from PBMC were treated with 10 U/ml IFNγ for 24 hours. CD86 expression was assessed by flow cytometry. Representative histogram of 3 independent experiments. (B) Murine B cells from WT C57BL/6 or IFNγR deficient (IFNγR KO) mice were incubated with 5 μg/ml CD40 activating Ab±10 U/ml IFNγ. Expression of CD86 was assessed by flow cytometry. Representative histogram of 4 independent experiments. (C) Stepwise schema of B_{Vax} generation in-vitro. B_{Vax} are produced from 4-1BBL⁺ B cells isolated from spleen, deep and superficial cervical lymph nodes. Cells are then activated with 5 μg/ml CD40 activating Ab and supplemented with 100 nM of B-cell survival factor BAFF, which after 24 hours allows the up-regulation of CD86, H-2K^{b} and IFNγRI when compared with only BAFF treated B cells. Addition of IFNγ for additional 24 hours allowed further upregulation of CD86 when compared to only anti-CD40 treated B cells. Histograms represent mean±SD of n=3 mice/treatment. (D) B_{Vax} over-express both IA^{b} (MHC class II) and H-2K^{b} when compared to only BAFF treated B cells (B_{Naive}). Flow cytometry dot plot representative of n=4mice/group. (E) B-cell subsets (B_{Naive}+IFNγ and B_{Vax}) and DC were tested for their ability to present SIINFEKL (SEQ ID NO: 1) peptide to OT1 CD8⁺ T cells measured by cell proliferation and expression of intracellular GzmB. Representative experiment of 3 independent experiments performed in triplicates. (F) Same experiment as in (E) was performed using in addition CD8⁺ T cells from WT C57BL/6 mice as negative control. For all experiments shown in this figure, differences among multiple groups were evaluated using One-way ANOVA with post hoc Tukey's test and histograms are shown as mean±SD. In all experiments, statistical significance is depicted as ns: not statistically significant, *p<0.05, **p<0.01, ***p<0.001, ****p<0.0001.

FIG. 11 demonstrates that 4-1BBL is a key marker for B_{Vax} therapeutic effect. (A) In B-cell deficient mice, B_{Naive} and B_{Vax} were detected in draining dCLN and the circulation and B_{Vax} were found in the tumor-bearing brains. Histograms represent mean±SD of n=5 mice/group. (B) CD45.1⁺ B_{Naive} and B_{Vax} were intracranially injected using cannula-guided injections 10 days after tumor implantation. Four days after, CD45.1⁺ cells were magnetically isolated from tumors, and tested for their ability to activate CD8⁺ T cells, measured by cell proliferation (Expansion index) by flow cytometry. Histograms represent mean±SD of n=3 mice/group. (C) B_{Naive}, B_{Act} and B_{Vax} were tested for their therapeutic effect in CT2A-bearing B-cell deficient mice (n=9-10 mice/group). (D) CT2A-bearing B-cell deficient mice treated with B_{Vax} (treated with 4-1BBL blocking Ab)±4-1BBL blocking Ab (i.p. 500 μg/mouse×3 injections after B_{Vax} adoptive transfer) were monitored for survival (n=10 mice/group).For all experiments shown in this figure, differences among multiple groups were evaluated using One-way ANOVA with post hoc Tukey's test and histograms are shown as mean±SD. Survival curves were generated via the Kaplan-Meier method and compared by log-rank test and multiple comparisons were adjusted using Bonferroni method. In all experiments, statistical significance is depicted as ns: not statistically significant, *p<0.05, **p<0.01, ***p<0.001, ****p<0.0001.

FIG. 12 demonstrates the therapeutic effect of B_{Vax}. (A) Radiotherapy (RT) induce systemic leukopenia measured by CD45⁺ leukocytes in blood and in the spleen (n=4 mice/group). (B) Histological evaluation of CT2A tumor burden at different time points (9 and 15 days after tumor implantation). All mice received 9Gy radiation 7 days after tumor inoculation. (C) B_{Vax} therapeutic effect was evaluated in animal that received radiotherapy (RT) 7 days after tumor implantation (n=10 mice/group). (D) B_{Vax}+CD8⁺ T-cell treatment efficacy was tested at different time points: 9 and 15 days after tumor inoculation. All mice received radiotherapy 7 days after tumor injection (n=7 mice/group). For all experiments shown in this figure, differences among multiple groups were evaluated using One-way ANOVA with post hoc Tukey's test and histograms are shown as mean±SD. Survival curves were generated via the Kaplan-Meier method and compared by log-rank test and multiple comparisons were adjusted using Bonferroni method. In all experiments, statistical significance is depicted as ns: not statistically significant, *p<0.05, **p<0.01, ***p<0.001, ****p<0.0001.

FIG. 13 demonstrates that B_{Vax} treatment results in tumor eradication. (A) PD-L1 membrane expression was evaluated in B_{Vax} and B_{Naive} cells. Representative dot plot of 4 independent experiments. (B) CD8⁺ T-cell infiltration in CT2A tumor in control group (14 days after tumor inoculation, n=2). Number of CD8⁺ T cells per section was assessed in the tumor area (tumor) vs non-tumor (brain) area. (C) Immune cell infiltration in long-term survivors treated with either B_{Vax} and CD8⁺ T cells (LTS-B_{Vax}+CD8, n=5) or only CD8⁺ T cells (LTS-CD8, n=4) were compared to control CT2A-bearing (Control, n=3) and mock brains (No tumor, n=2). Representative dot plot showing lymphocyte and myeloid cell compartment distribution (CD45 and CD11b expression) and CD8⁺ and CD4⁺ T-cell distribution. Within the CD4⁺ T-cell compartment, the expression of CD44 and Foxp3 was evaluated. Within the non-T-cell compartment (CD8⁻CD4⁺ lymphocytes), the CD19⁺ B-cell compartment expressing IFNγ and 4-1BBL was evaluated. Values of the populations of interest are shown as mean±SD. GBM patient-derived B_{Vax} promote anti-tumor CD8⁺ T cells. (D) Schema of generation of GBM patient-derived B_{Vax}. CD8⁺ T-cell activation and expansion and CD8⁺ T-cell-mediated tumor cell killing assay was performed in autologous settings. (E) Freshly resected tumors from GBM patients were cultured ex-vivo as tumor spheroids. Representative picture of adherent cells after 5 days of culture. Bars represent 100 μm.

FIG. 14 shows B-cell receptor sequence analysis. (A) Immunoglobulin heavy chain (IgH) DNA sequence was analyzed in B_{Vax} and compared to naïve B cells (Bₙₐᵢᵥₑ) and tumor-infiltrating (TI) B cells. Data is shown as frequence of clones. Statistical significance is highlighted in blue or red spots. (B) Three clones are significantly enriched in B_{Vax} compared to B_{Naive} and TI B cells (gray; top three listed). Six clones are significantly enriched in B_{Vax} compared to B_{Naive} and overlap with clones that are highly abundant in TI B cells (blue; bottom 6 in list). Figures are representative of 3 independent experiments.

FIG. 15 shows that B_{Vax} differentiate into plasmablasts in tumors. (A) B_{Vax} obtained from CD45.1⁺CT2A-bearing mice were injected intravenously into CD45.2⁺ CT2A-bearing mice (T) and naïve mice (NT). After 72 hours mice were sacrificed and analyzed for the presence of CD45.1⁺ cells in blood, tumor-bearing brains, deep cervical lymph nodes (dCLN) and the spleen. Only tumor-bearing brains harbored CD45.1⁺ B_{Vax}. (B) Plasmablast marker CD138 expression by CD45.1⁺ B_{Vax}. Histograms represents average±standard deviation of n=8 mice per group.

### DETAILED DESCRIPTION

Immunotherapy is a promising approach to treat glioblastoma (GBM) patients, yet its efficacy has so far been very limited. In the present application, the inventors demonstrate the ability of 4-1BBL+ B cells to promote anti-tumor immunity, and propose that these B cells may be used as a vaccine to treat deadly tumors, including glioblastoma.

To stabilize their antigen presentation function in vivo and avoid potential immunosuppressive functions, the present inventors prepared activated 4-1BBL⁺ B cells designated as B_{Vax}. The B cells were activated using CD40 and IFNγ receptor (IFNγR) ligation. In the present application, the inventors demonstrate that B_{Vax} inhibit GBM growth by promoting tumor-specific CD8⁺ T-cell immunity. Further, they demonstrate the therapeutic effectiveness of B_{Vax}, both alone and in combination with radiation and checkpoint blockade.

### Methods

The present invention provides methods of making an anti-cancer composition. The methods comprise: (a) collecting 4-1BBL+(CD137L+) B cells; (b) incubating the B cells with a CD40 agonist; (c) adding IFN-γ to the B cells; and (d) contacting the B cells with tumor-derived antigens.

As used herein, the term “anti-cancer composition” refers to any substance that, when administered in a therapeutically effective amount to a subject suffering from cancer, provides a therapeutic benefit such as (1) curing the cancer; (2) slowing the progress of the cancer; (3) causing the tumor to regress; or (4) alleviating one or more symptoms of the cancer.

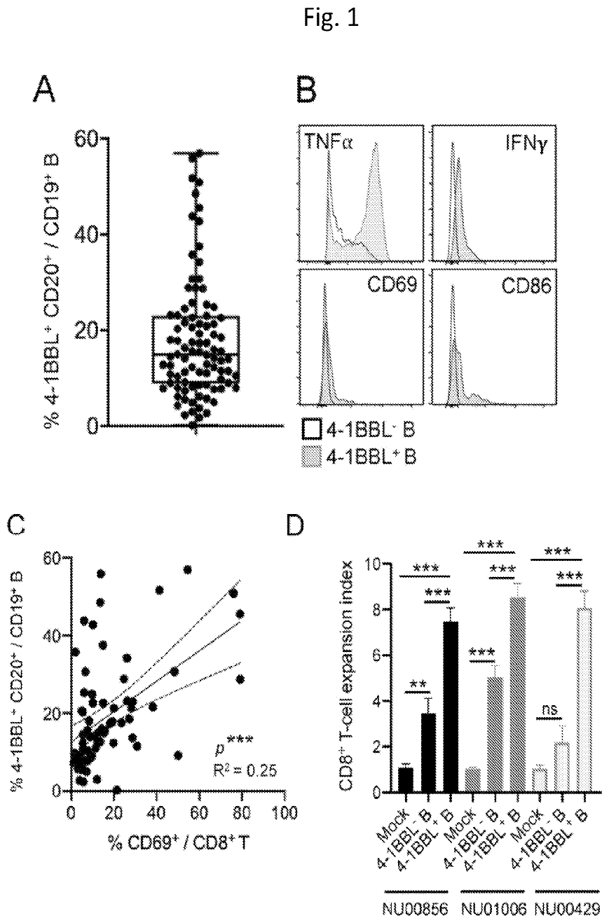

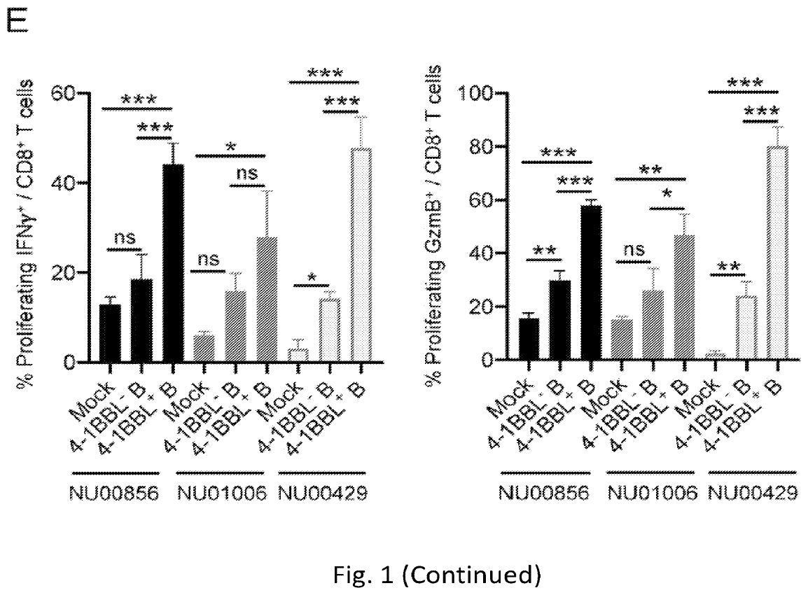

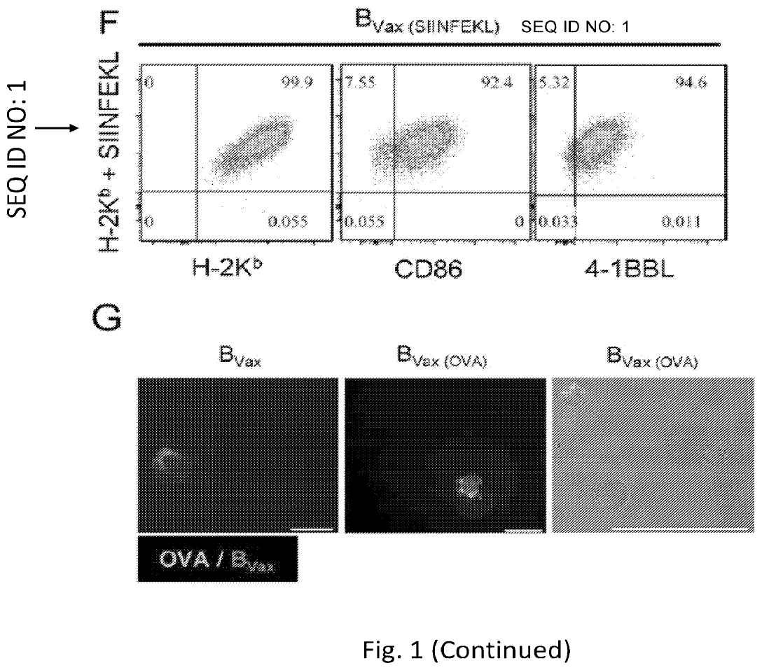

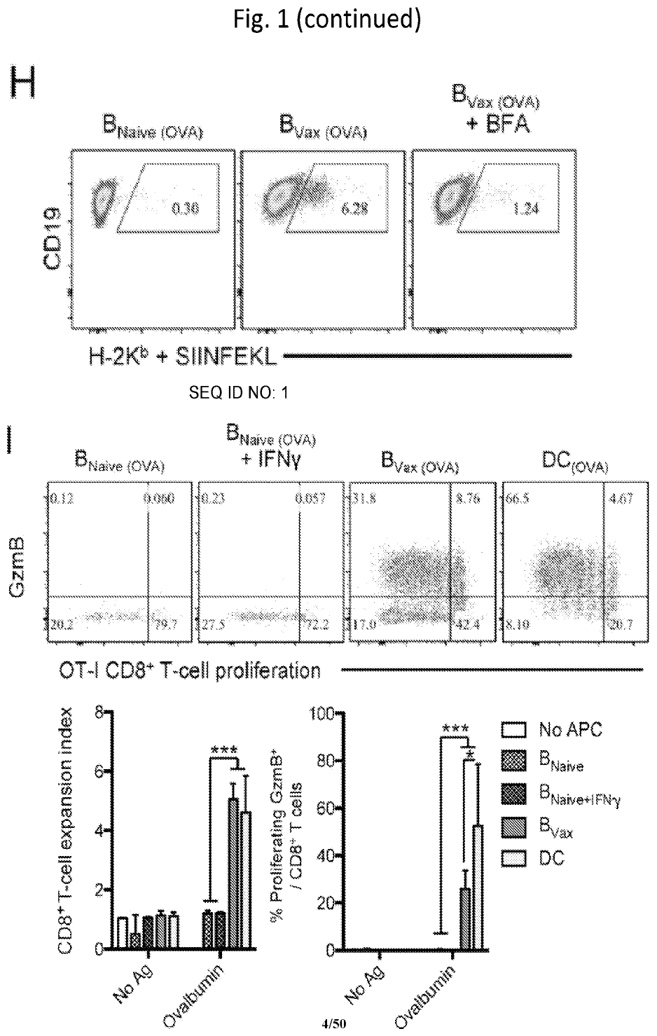

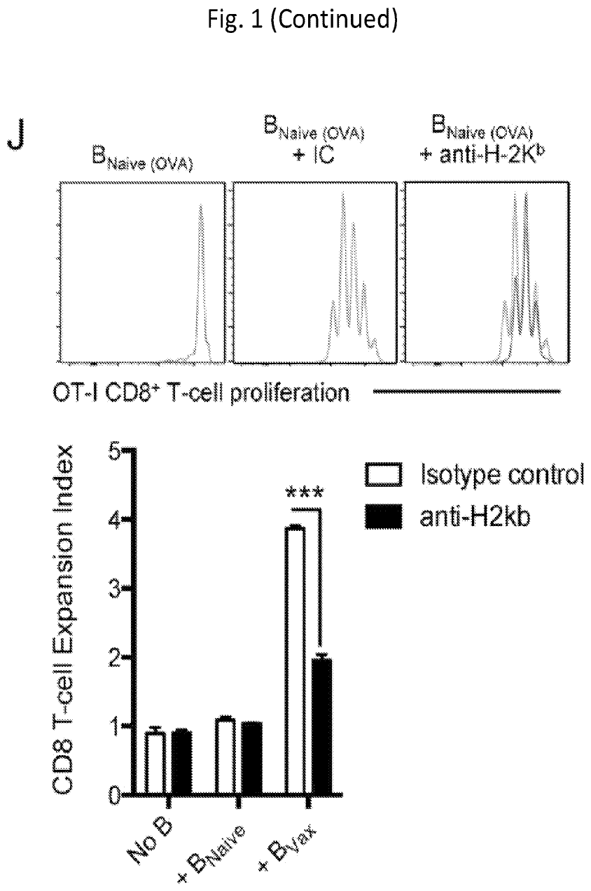

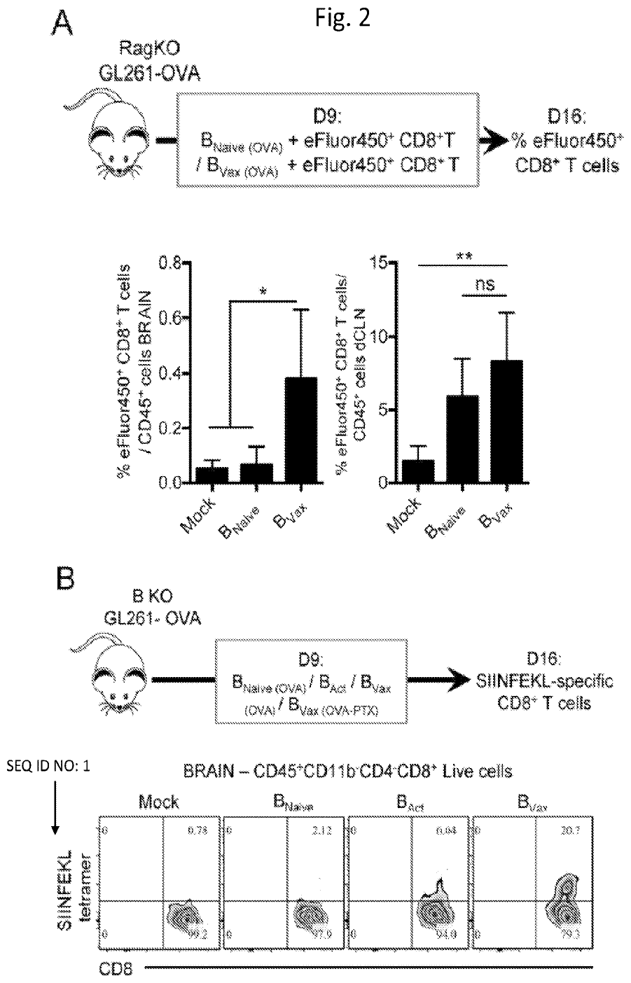

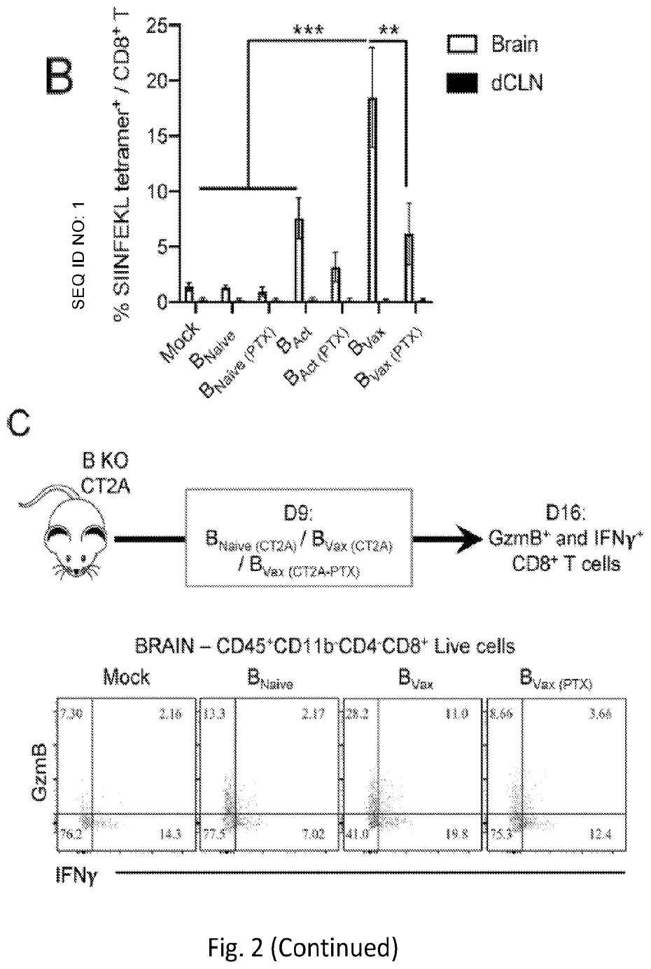

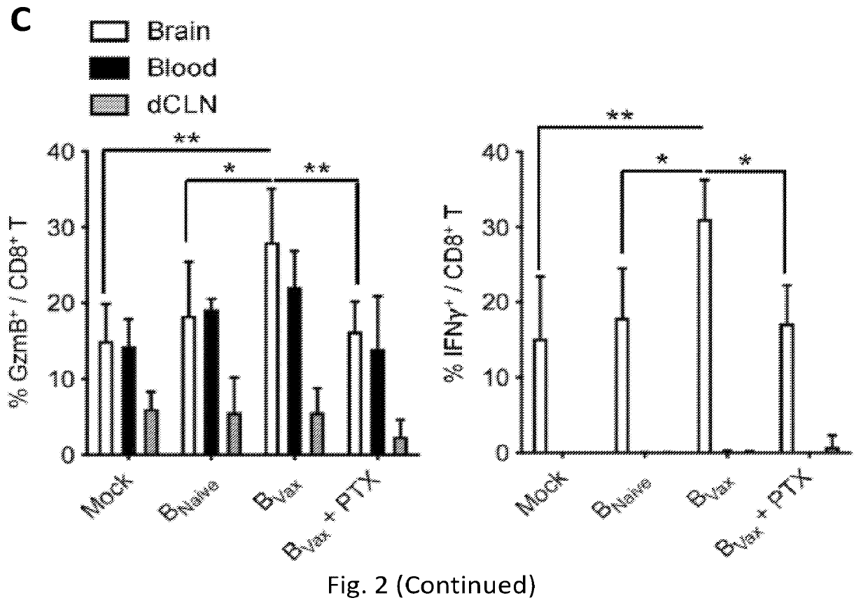

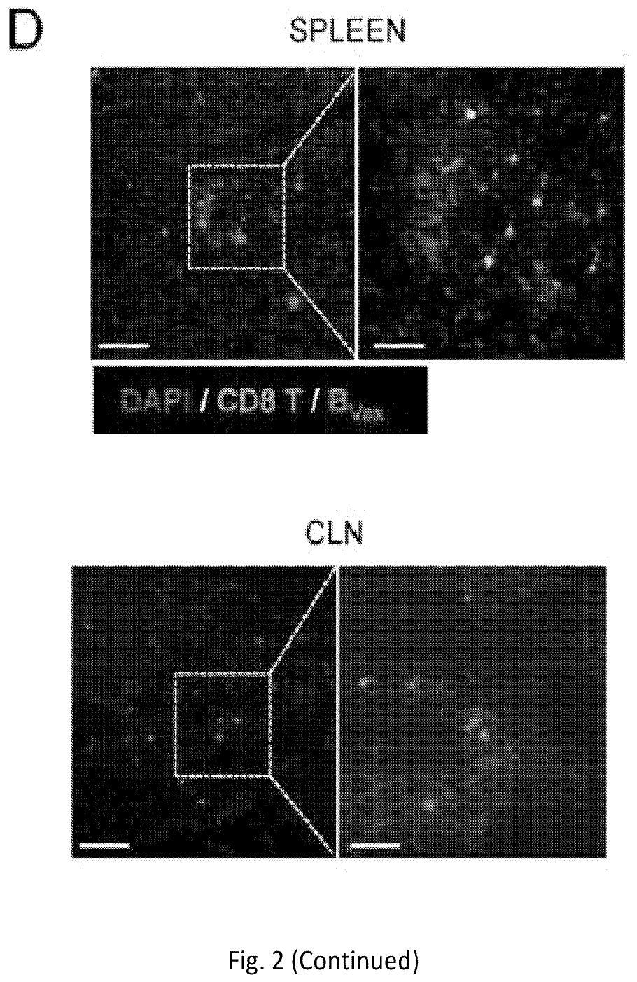

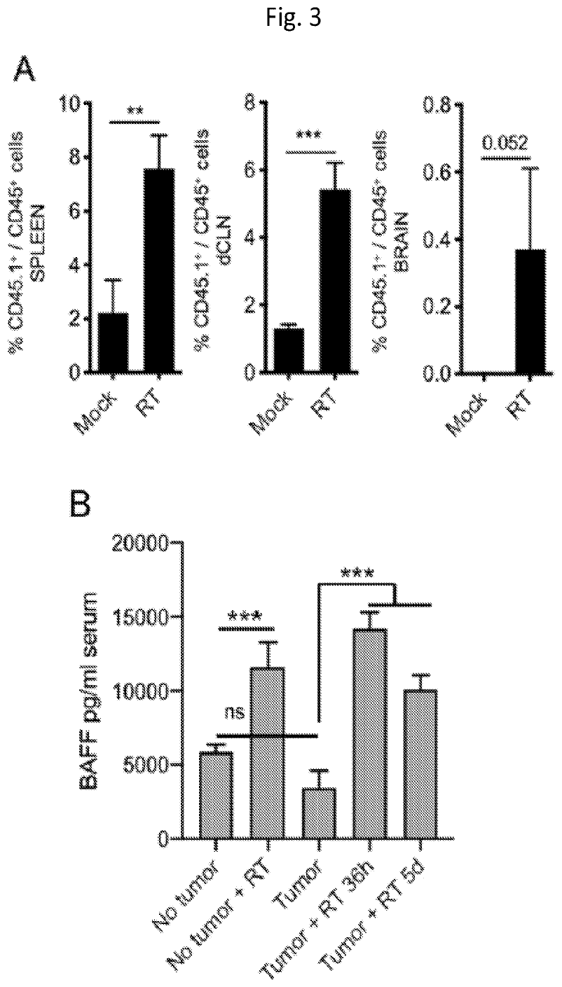

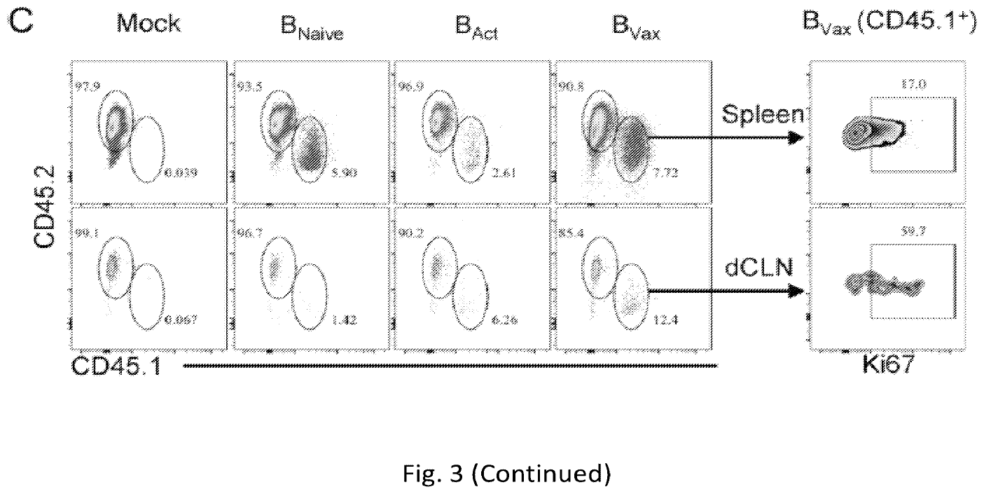

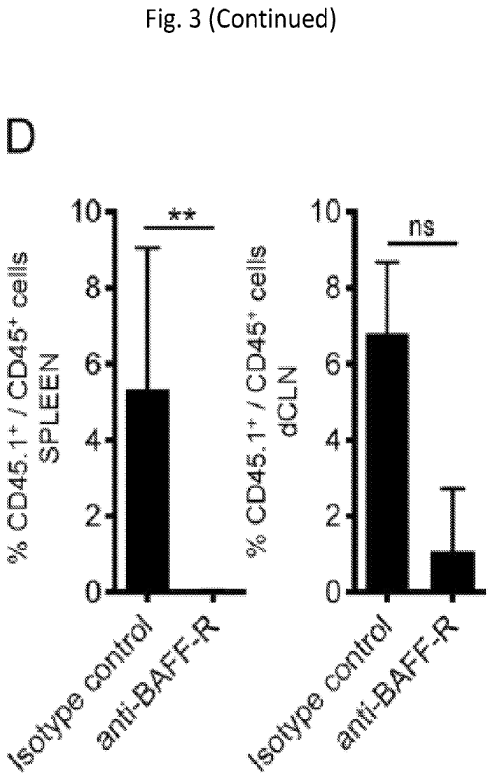

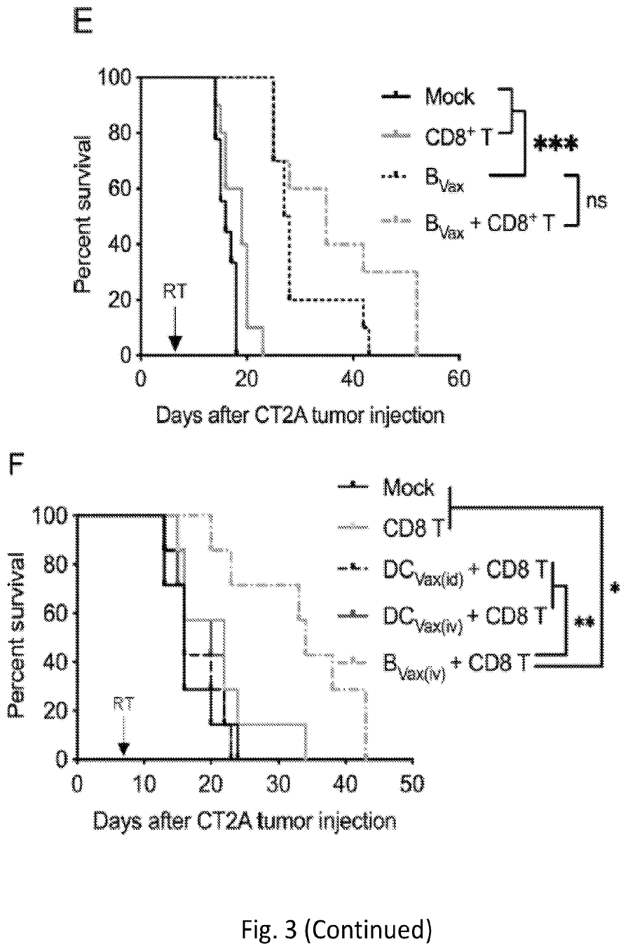

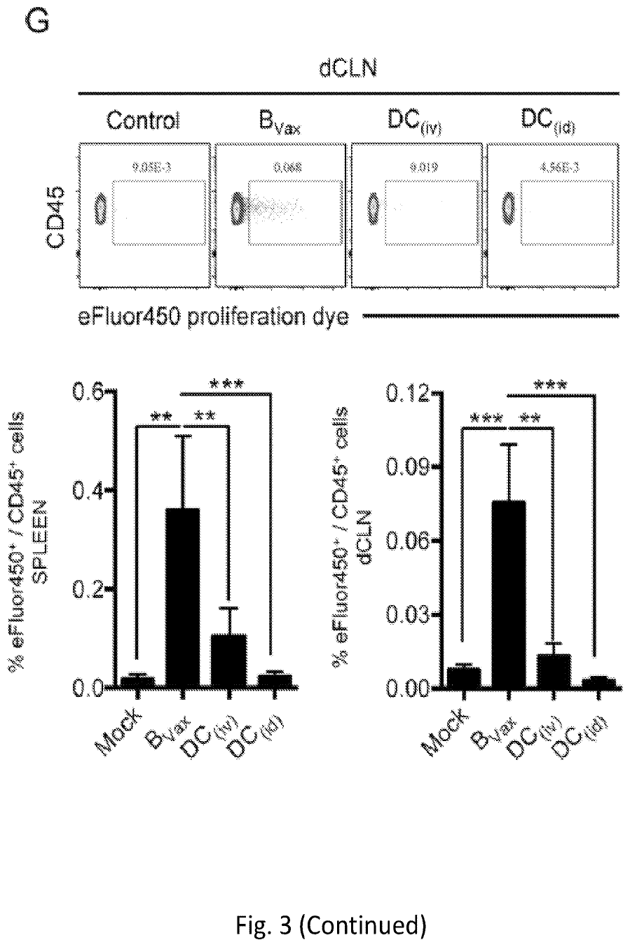

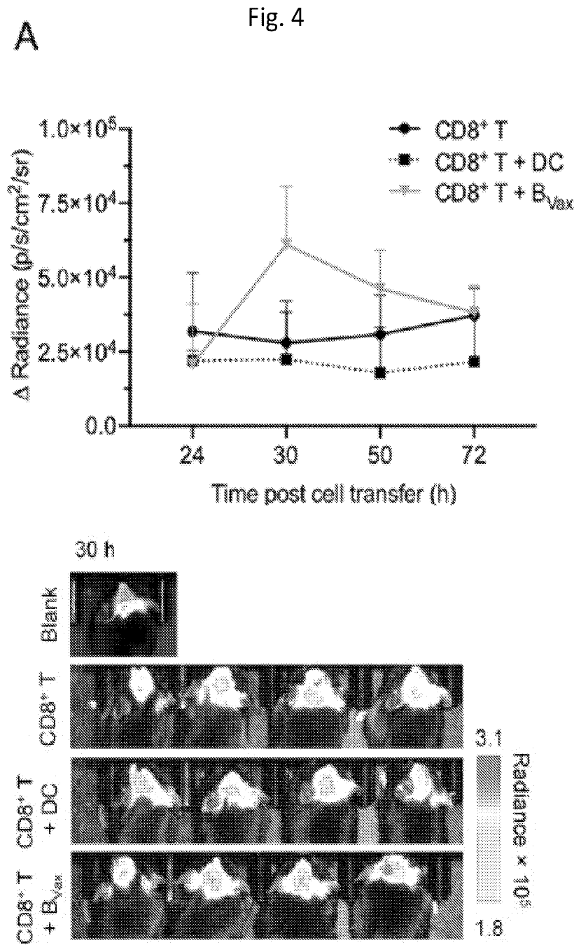

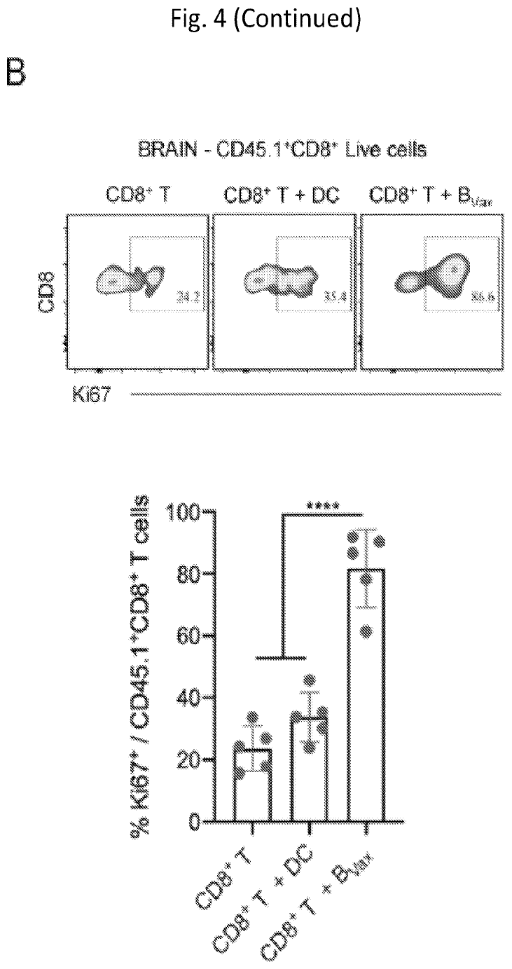

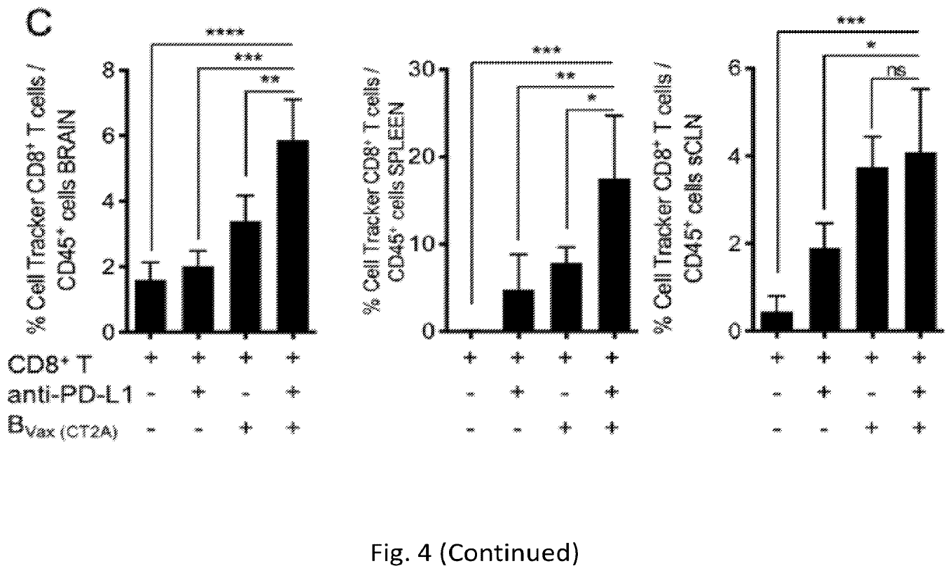

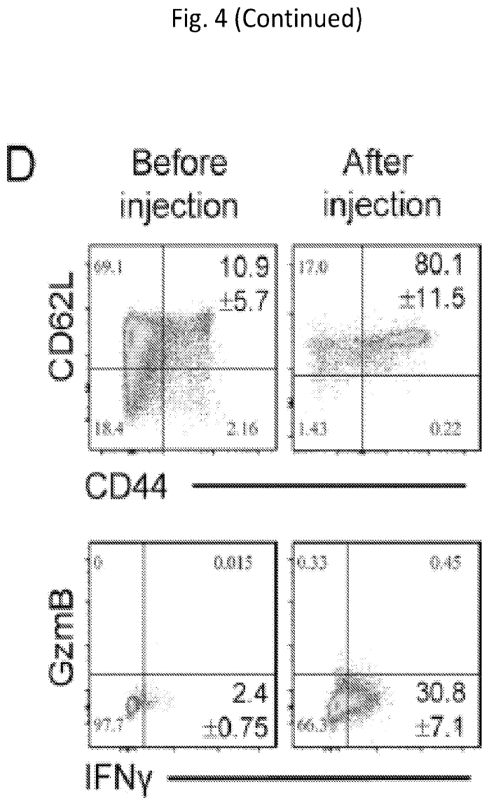

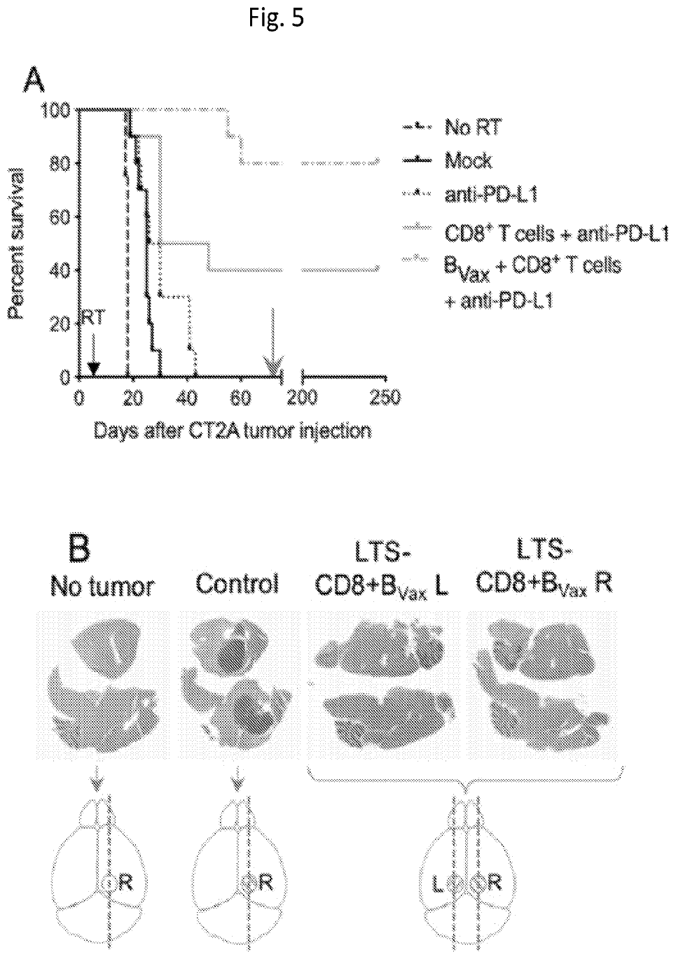

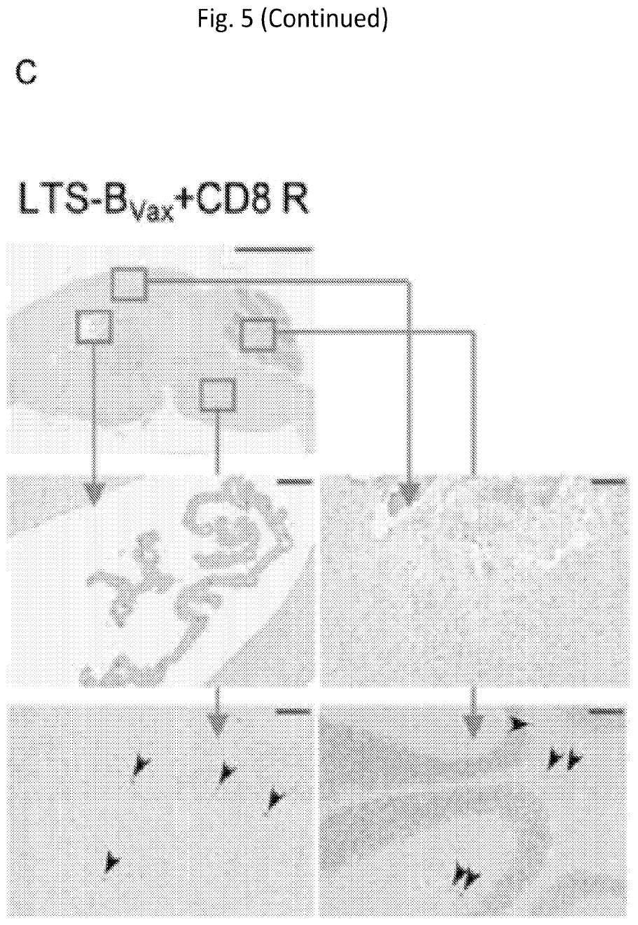

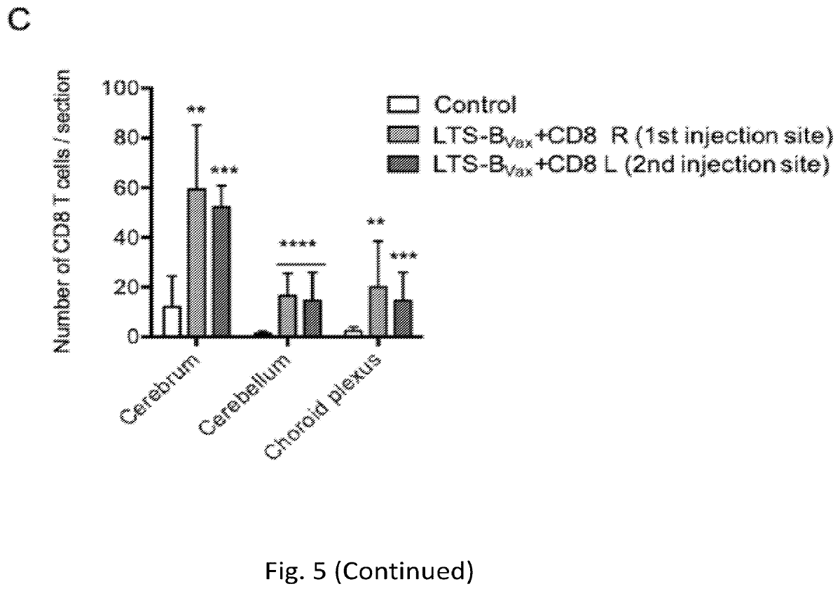

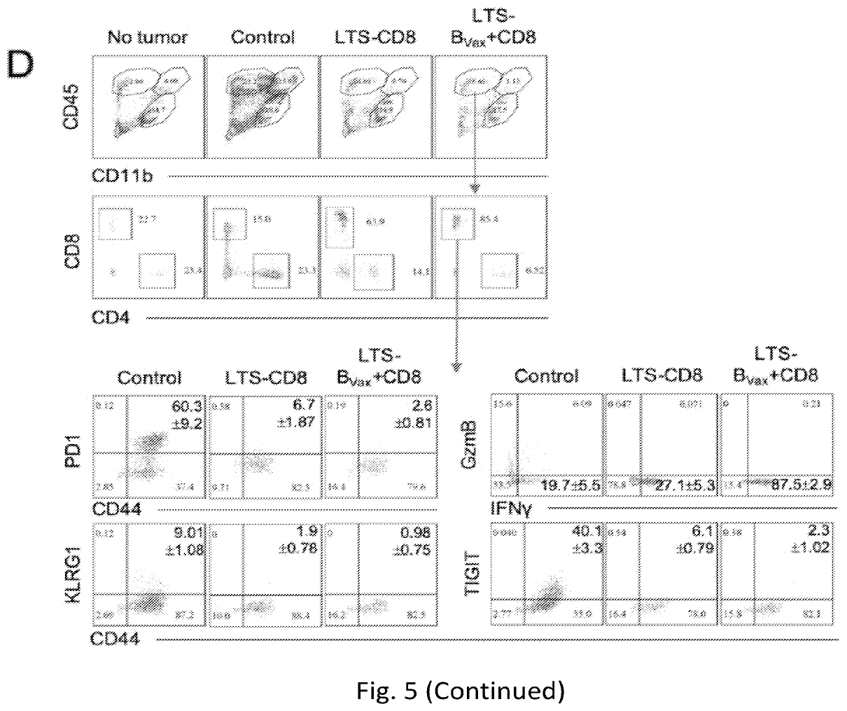

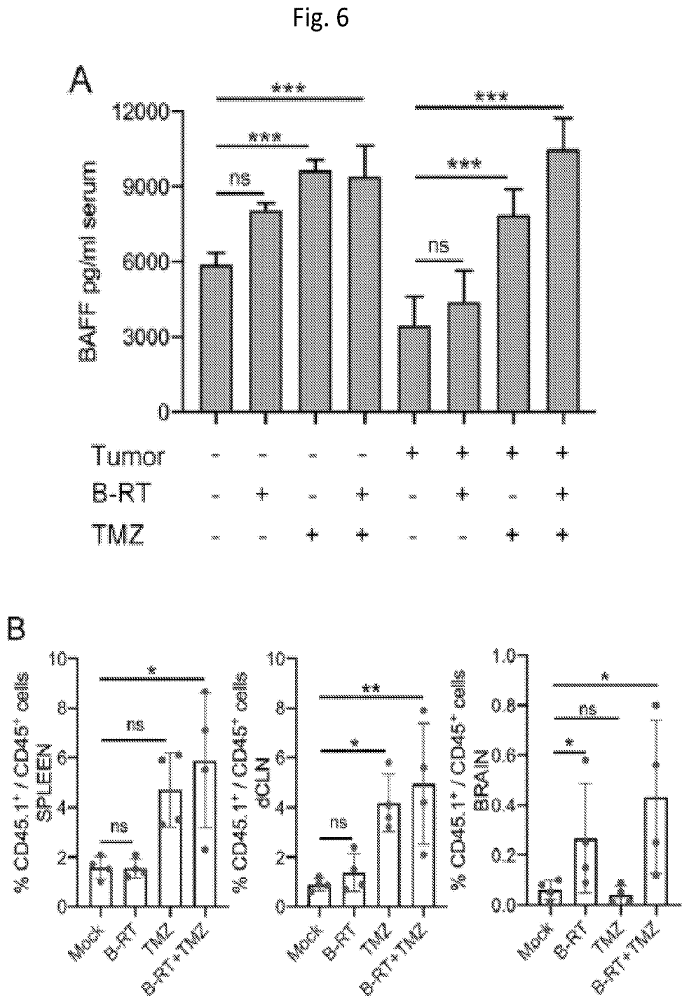

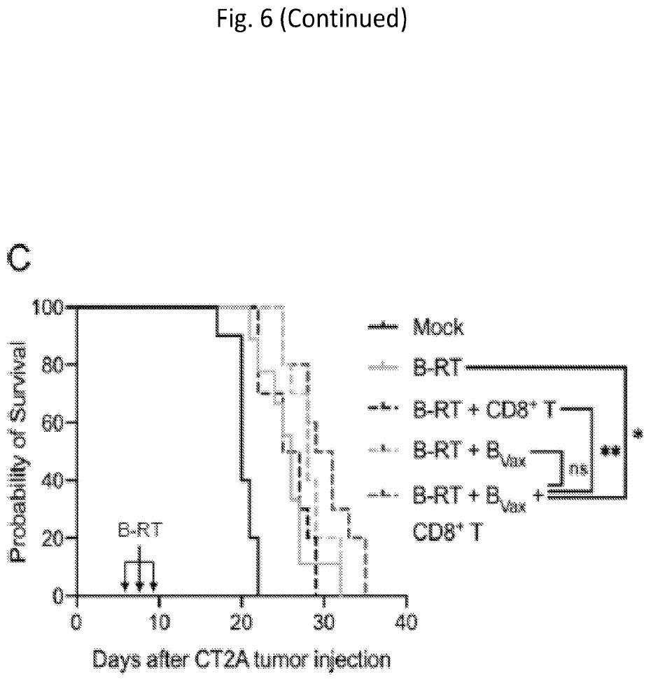

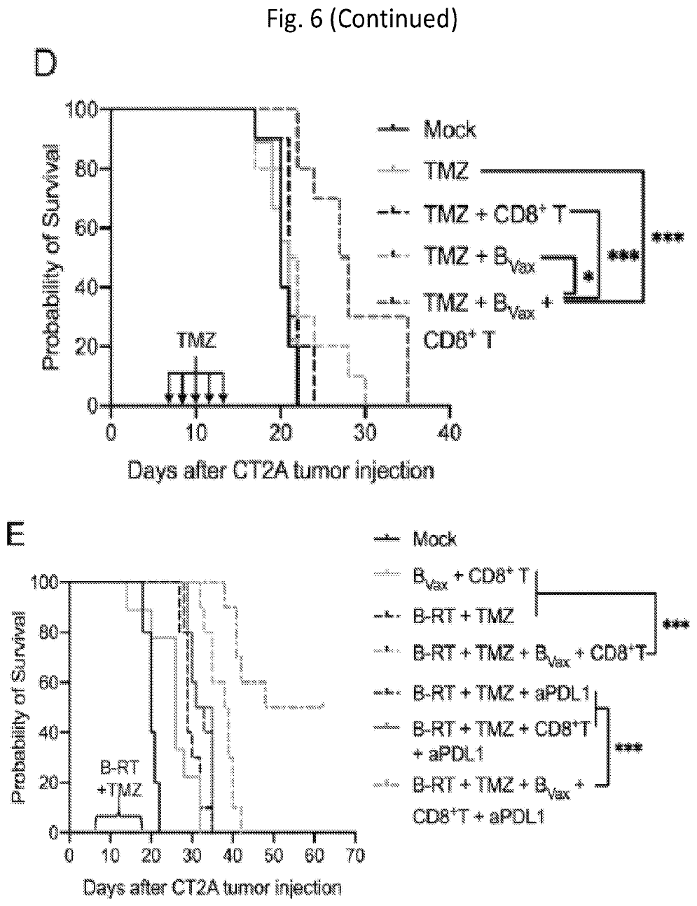

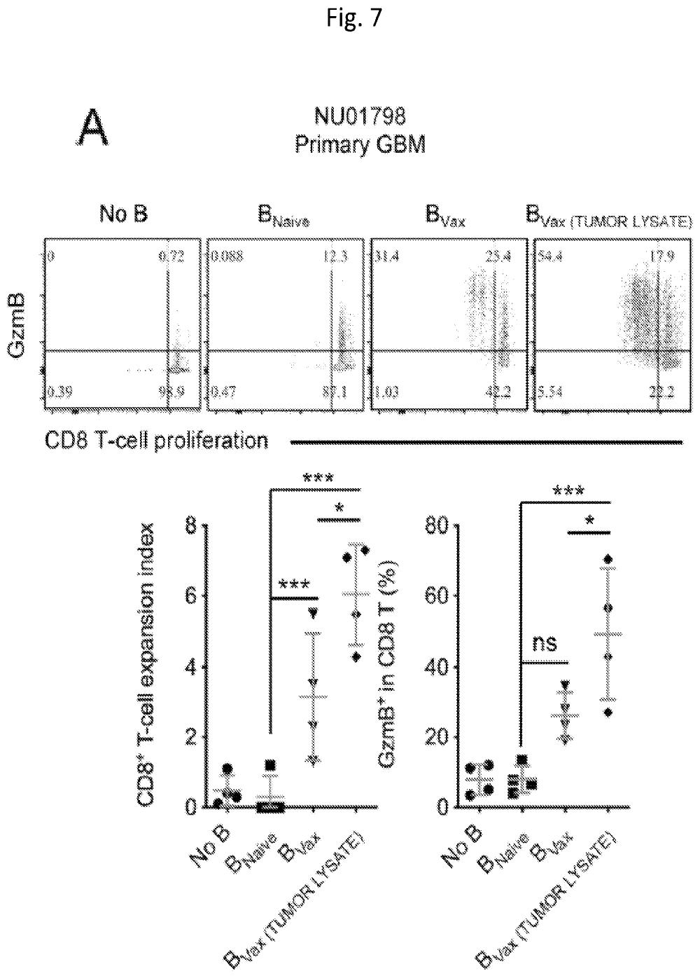

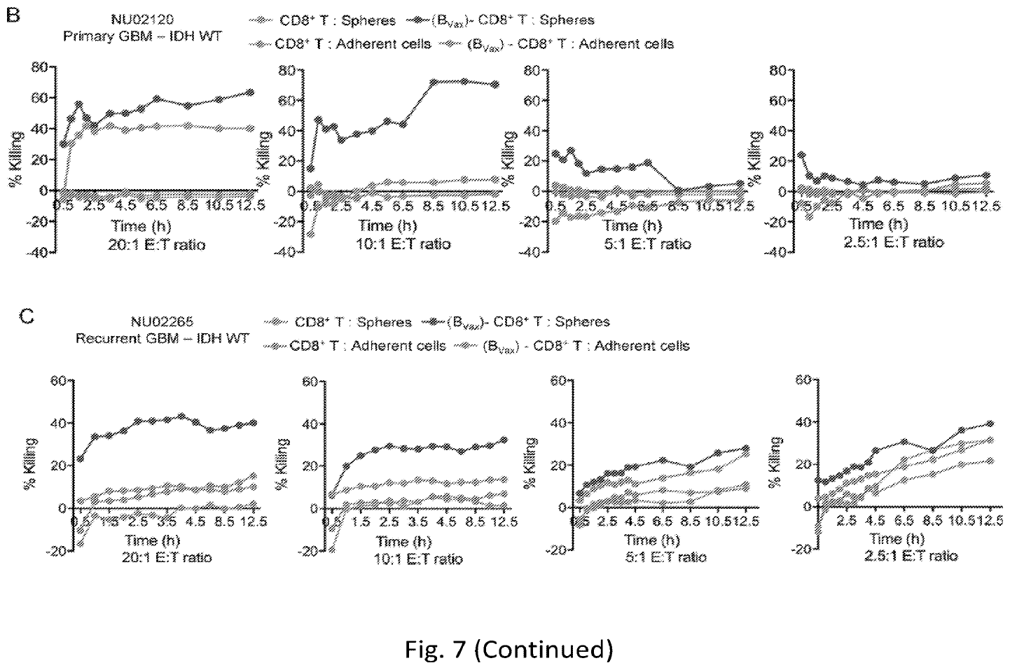

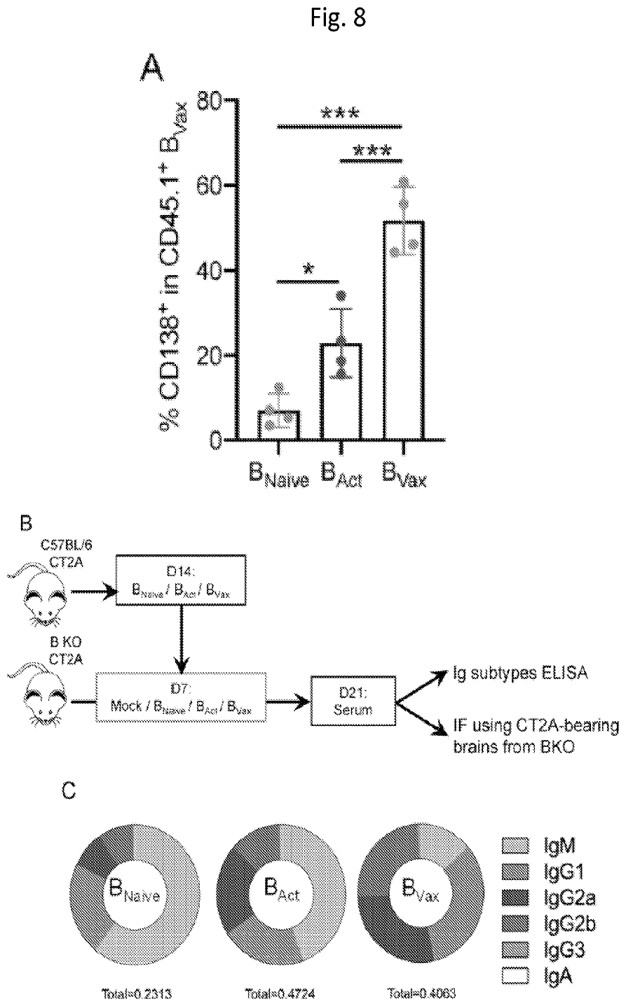

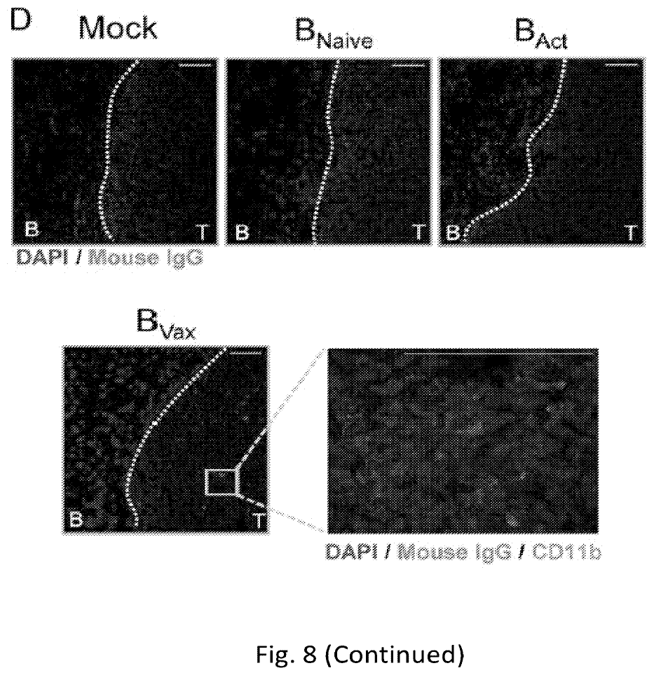

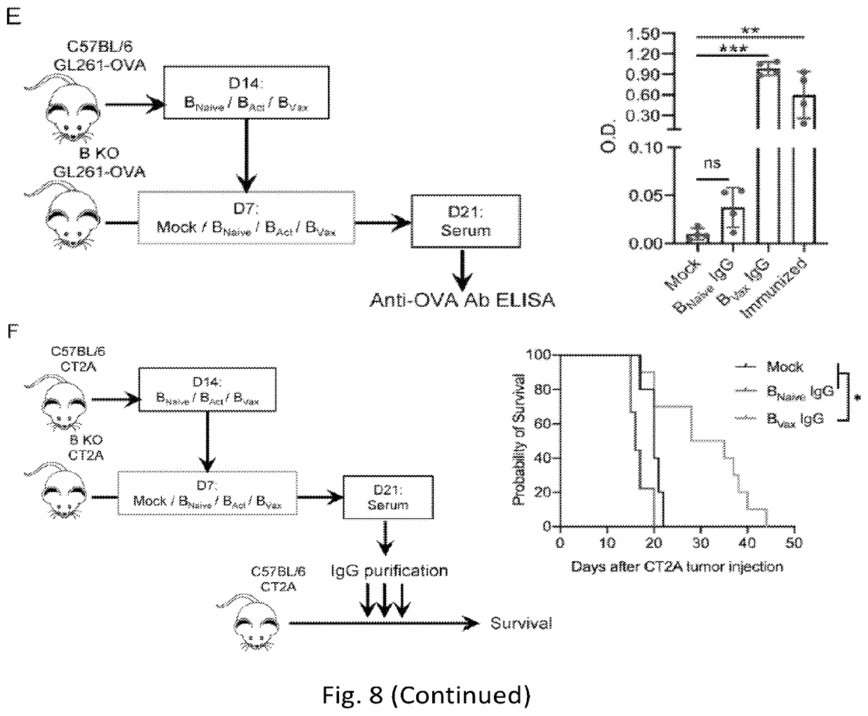

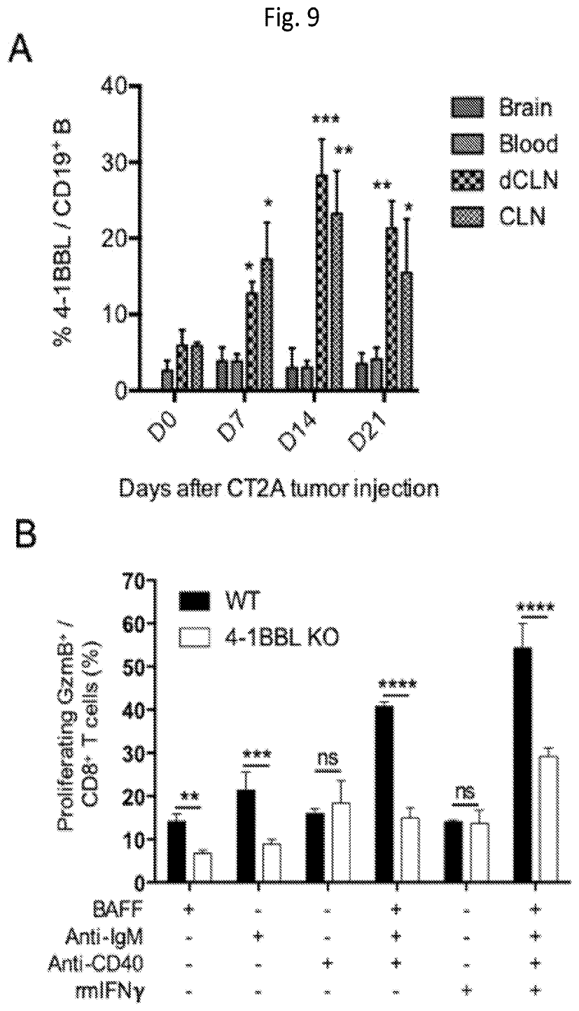

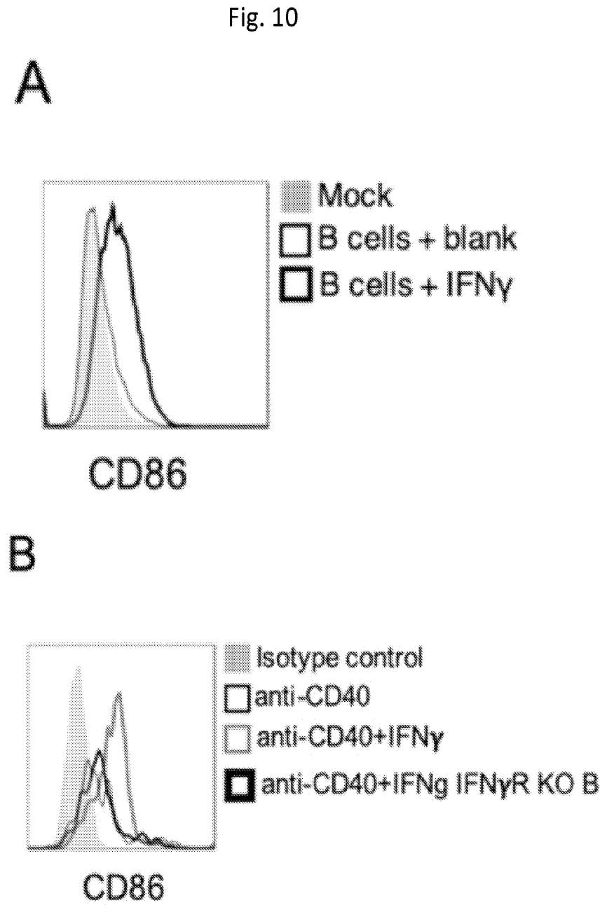

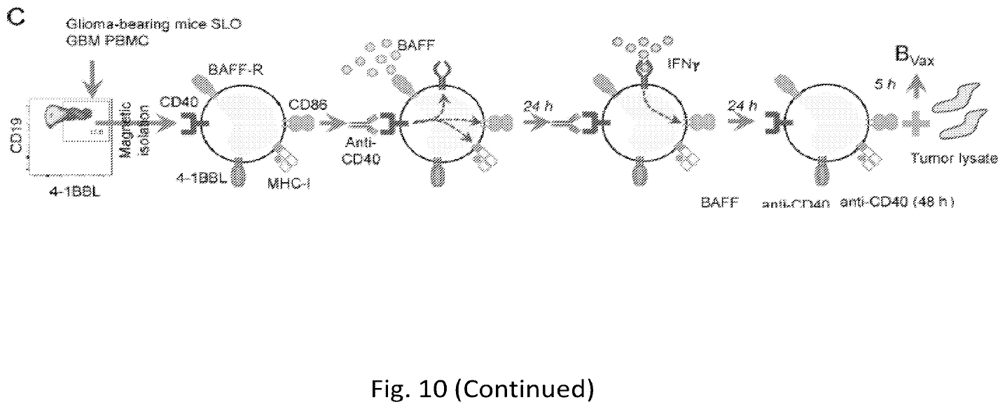

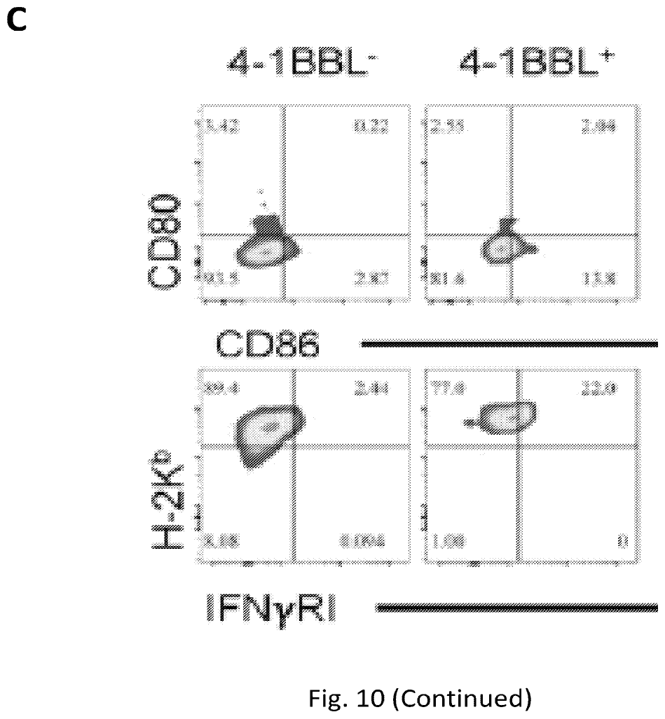

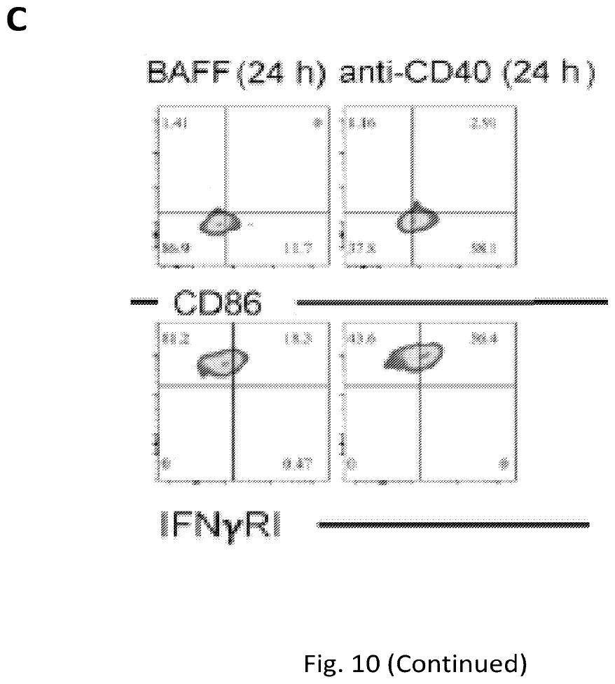

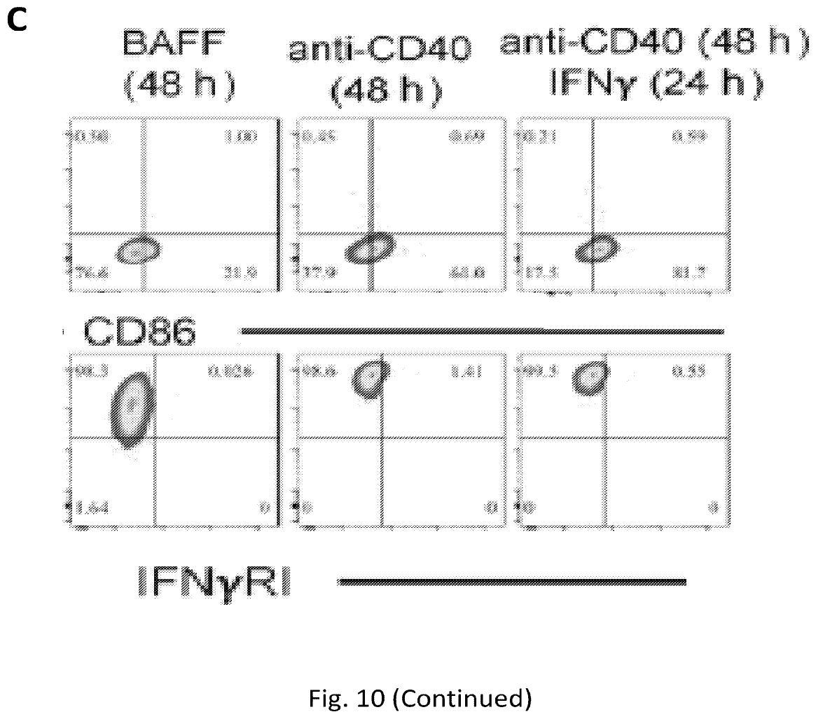

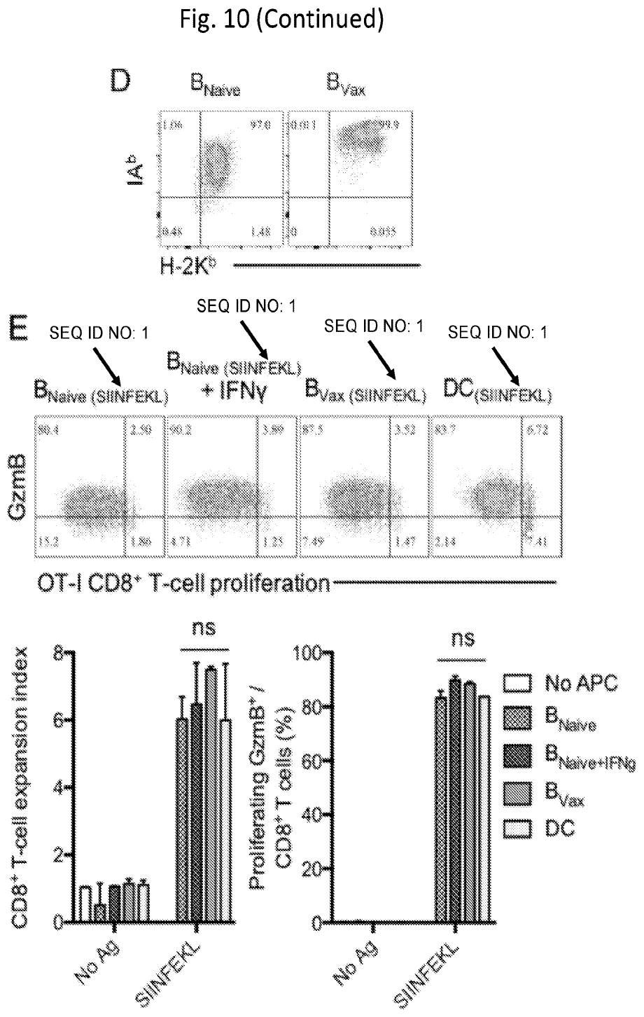

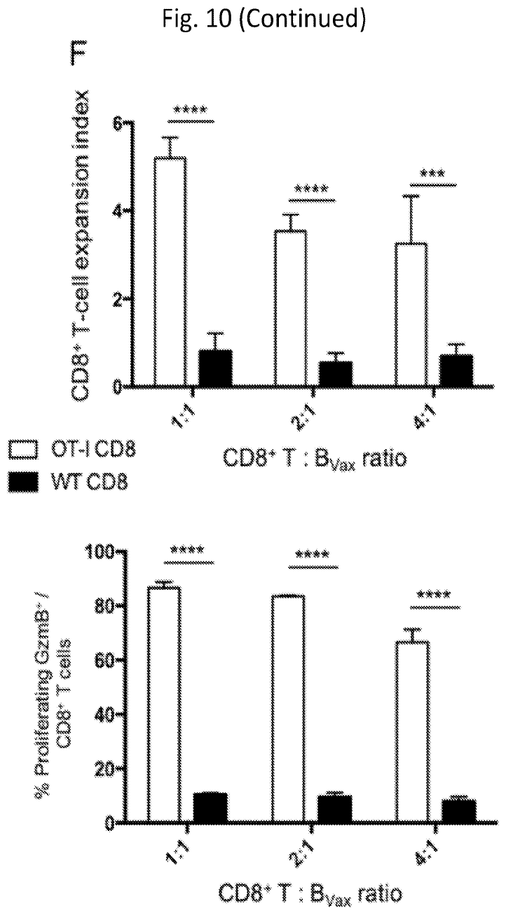

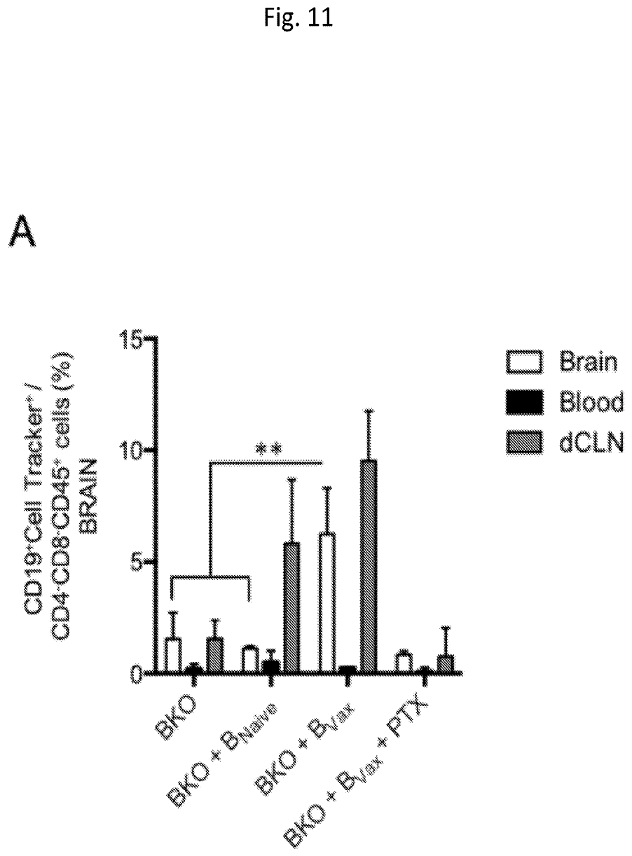

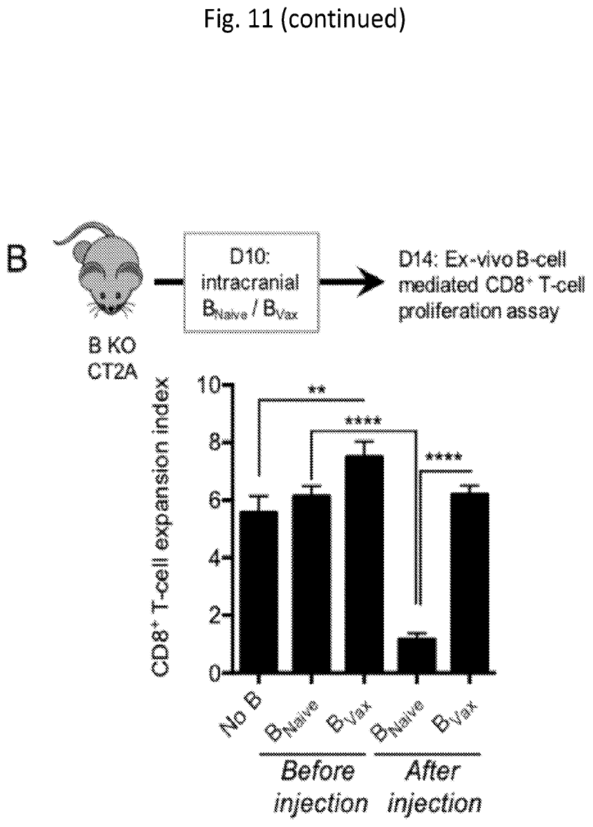

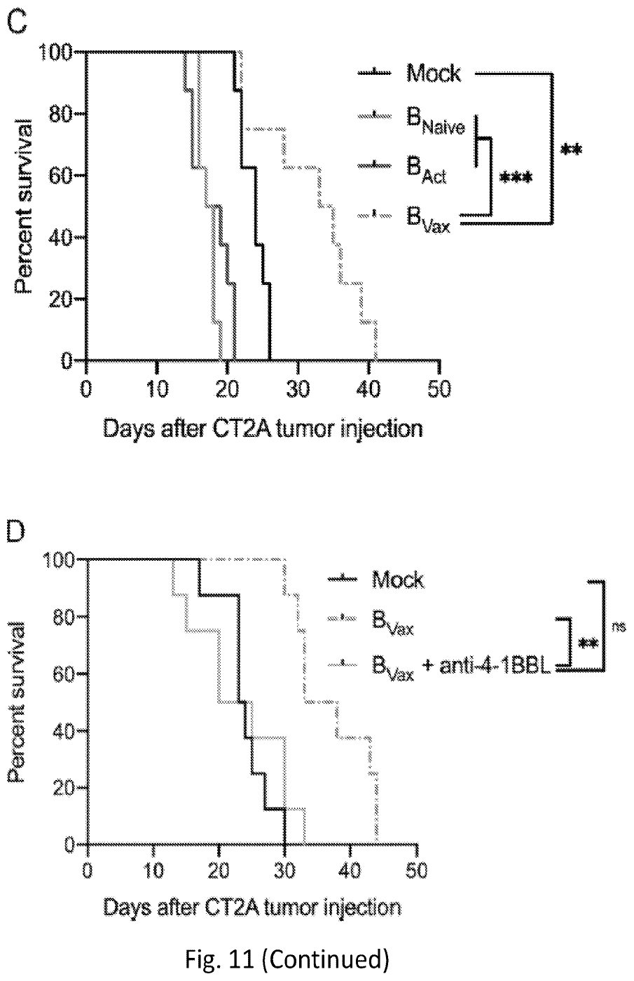

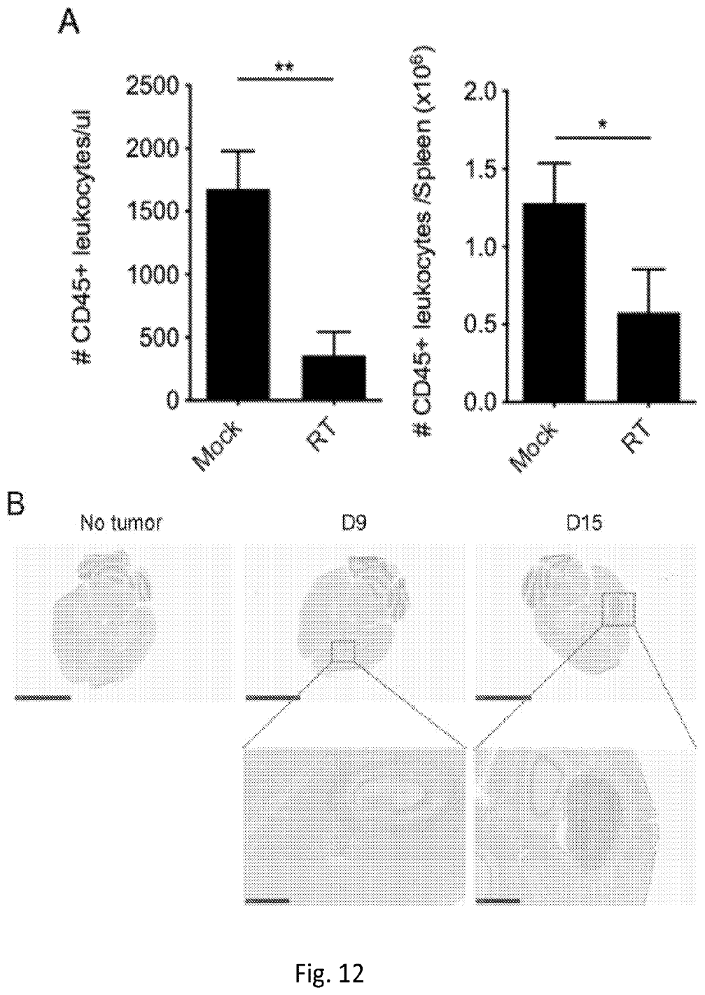

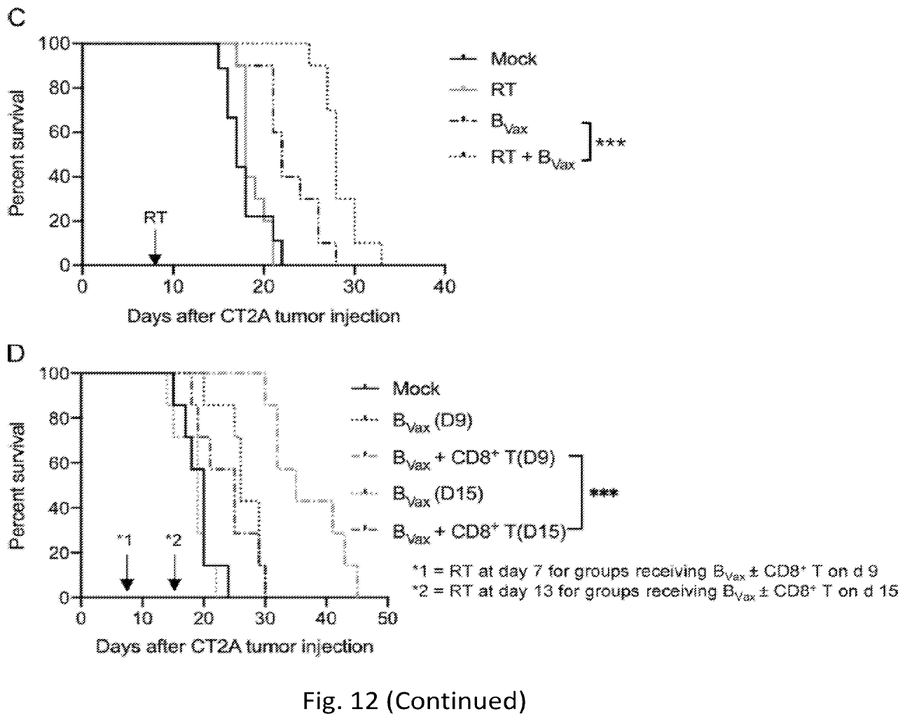

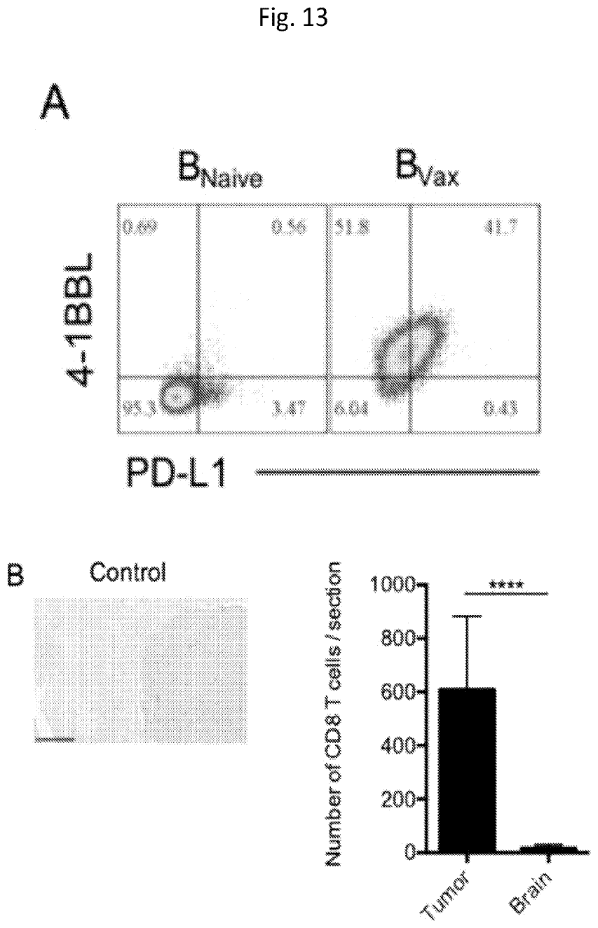

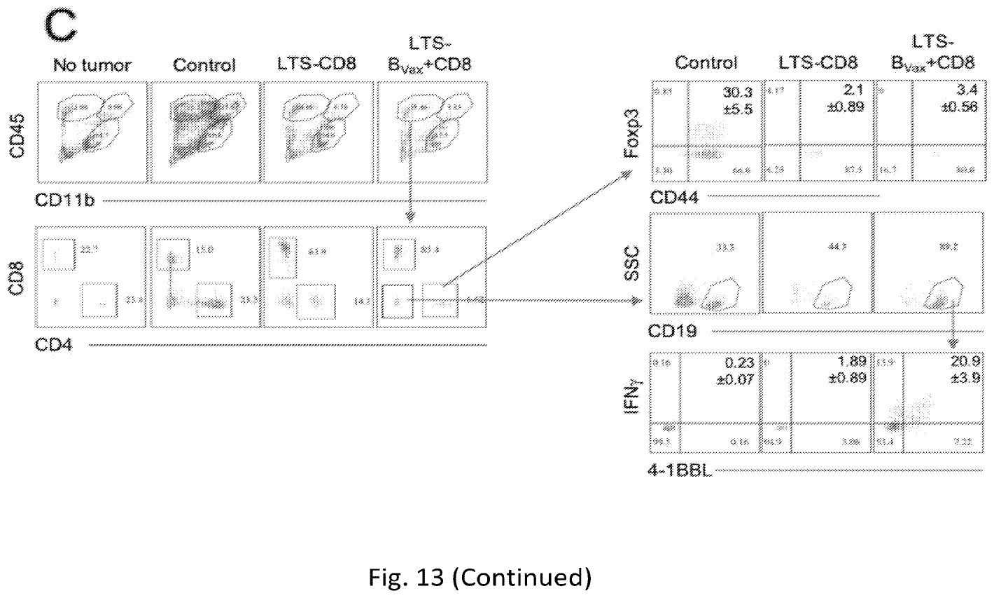

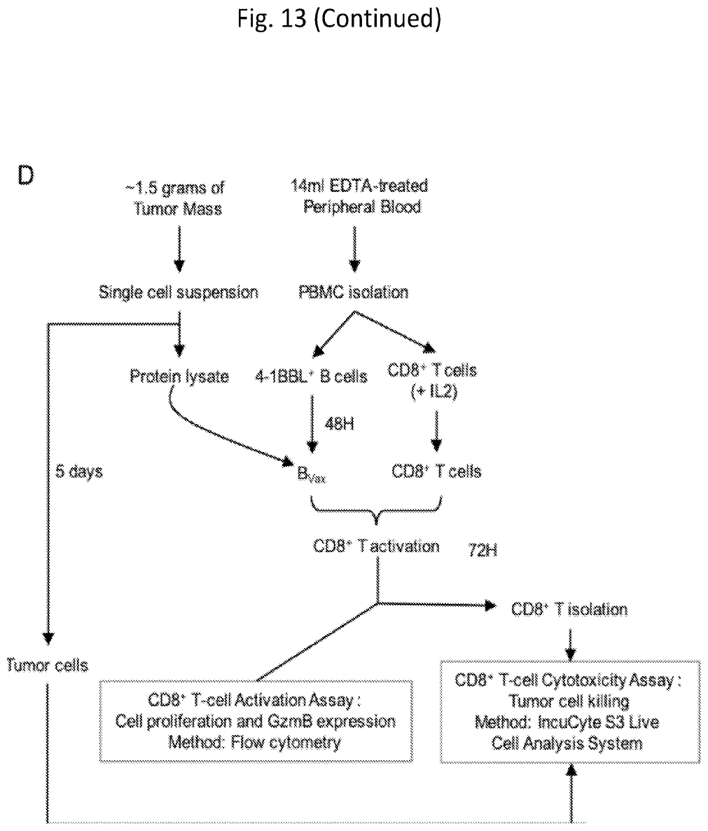

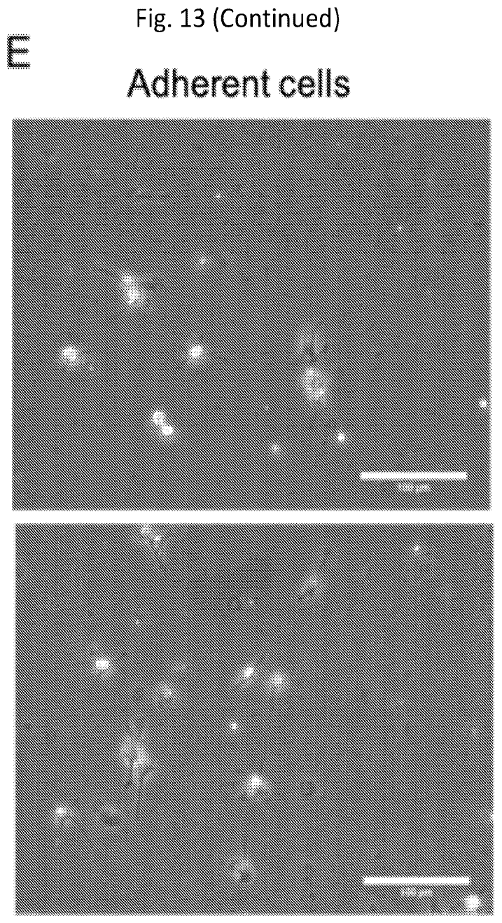

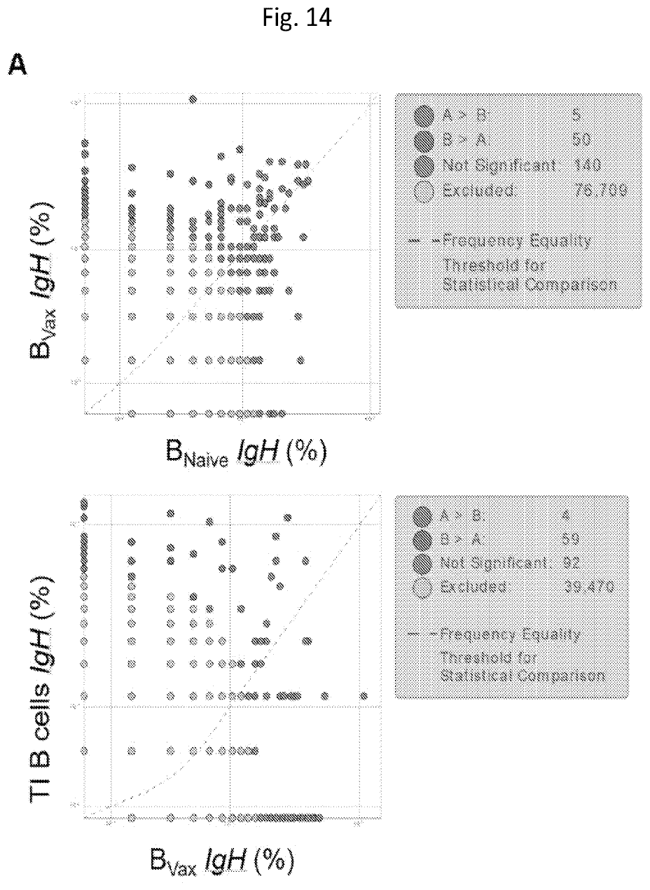

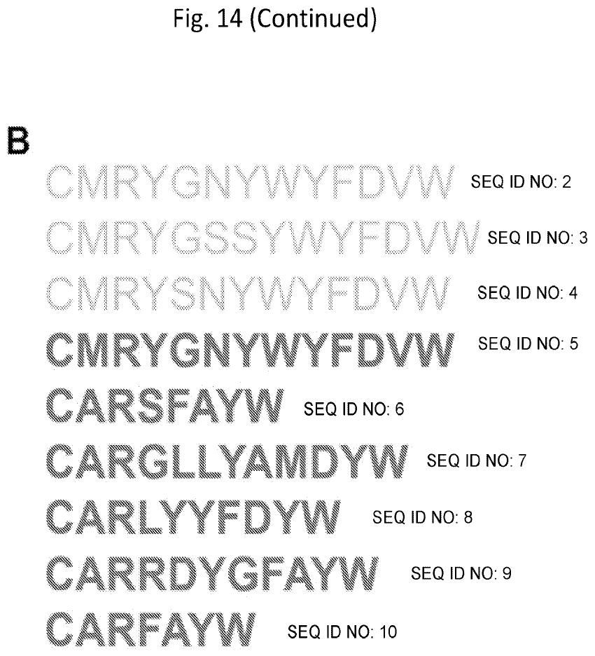

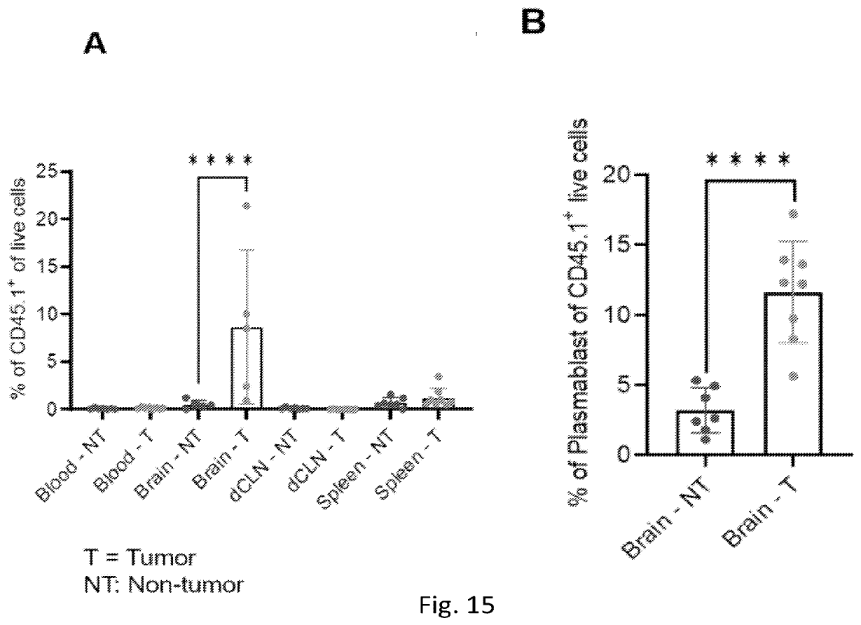

The present invention utilizes 4-1BBL+ B cells as a cellular platform. 4-1BB (also known as CD137L) is a member of the tumor necrosis factor (TNF) receptor family and is a costimulatory molecule that is expressed on both activated T cells and B cells. Previous studies have shown that the subset of B cells that express this co-stimulatory marker enhance CD8⁺ T-cell anti-tumor cytotoxicity via multiple mechanisms, including antigen presentation, T-cell co-stimulation (4-1BBL and CD86), and cytokine production (TNFα) (Lee-Chang et al., 2016; Lee-Chang et al., 2014). In the Examples, the inventors demonstrate that the 4-1BBL+ B cells of GBM patients have increased levels of activation markers and have greater ability to enhance CD8+ T-cell co-stimulation as compared to 4-1BBL− B cells (FIG. 1).

The anti-cancer compositions of the present invention are designed for use as an autologous therapy, i.e., a therapy in which the subject's own cells are modified outside the body and then reintroduced into the subject. A primary advantage of this approach is that it offers a lower risk of life-threatening complications due to immune rejection. Thus, in some embodiments, the 4-1BBL+ B cells used with the present invention are collected from a subject diagnosed with cancer.

The term “cancer” is meant to encompass any cancer, neoplastic and preneoplastic disease that is characterized by abnormal growth of cells. The cancer may be selected from the group consisting of colon carcinoma, breast cancer, pancreatic cancer, ovarian cancer, prostate cancer, head and neck cancer, lung cancer, Hodgkin's Disease, non-Hodgkin's lymphomas, rectum cancer, urinary cancers, uterine cancers, oral cancers, skin cancers, stomach cancer, brain tumors, liver cancer, laryngeal cancer, esophageal cancer, mammary tumors, fibrosarcoma, myxosarcoma, liposarcoma, chondrosarcoma, osteogenic sarcoma, chordoma, angiosarcoma, endotheliosarcoma, Ewing's sarcoma, squamous cell carcinoma, basal cell carcinoma, adenocarcinoma, sweat gland carcinoma, sebaceous gland carcinoma, papillary carcinoma, papillary adenocarcinomas, cystandeocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, endometrial cancer, lung carcinoma, small cell lung carcinoma, bladder carcinoma, epithelial carcinoma, glioblastomas, neuronomas, craniopharingiomas, schwannomas, glioma, astrocytoma, meningioma, melanoma, neuroblastoma, retinoblastoma, leukemias and lymphomas, acute lymphocytic leukemia and acute myelocytic polycythemia vera, multiple myeloma, Waldenstrom's macroglobulinemia, and heavy chain disease, acute nonlymphocytic leukemias, chronic lymphocytic leukemia, chronic myelogenous leukemia, childhood-null acute lymphoid leukemia (ALL), thymic ALL, B-cell ALL, acute megakaryocytic leukemia, Burkitt's lymphoma, and T cell leukemia, small and large non-small cell lung carcinoma, acute granulocytic leukemia, germ cell tumors, endometrial cancer, gastric cancer, hairy cell leukemia, thyroid cancer and other cancers known in the art. In some embodiments, the cancer is selected from the group consisting of glioblastoma, melanoma, breast cancer and pancreatic cancer. In a preferred embodiment, the cancer is a glioblastoma.

In the Examples, the inventors used a combination of a CD40 agonist and IFN-γ to produce activated B cells, designated as B_{Vax}, which have superior antigen-presenting cell (APC) function (FIG. 2) and are resistant to tumor immunosuppression in vivo (FIG. 3). Thus, in the present methods, the B cells are incubated with both a CD40 agonist and IFN-γ. The CD40 agonist is generally added prior to the IFN-γ as the CD40 agonist induces the expression of the IFN-γ receptor. The CD40 agonist is added for 18-24 hours prior to addition of the IFN-γ in one embodiment.

As used herein, the term “CD40 agonist” refers to a reagent that specifically binds to a CD40 molecule and induces CD40 signaling. CD40 is a transmembrane protein receptor that is expressed by antigen-presenting cells (APCs) and is involved in co-stimulation of immune cells. Current strategies to induce CD40 signaling include both antibody-based and CD40 ligand-based approaches. Thus, in some embodiments, the CD40 agonist is selected from CD154 (i.e., the cognate ligand for CD40) and a CD40 antibody or portion thereof capable of agonizing CD40. The B cells may be incubated with the CD40 agonist for up to 48 hours prior to addition of tumor-derived antigen. In certain embodiments, the B cells are incubated with the CD40 agonist for at least 12 hours prior to the addition of IFN-γ and as long as 48 hours. In the Examples, a CD40 antibody is used as the CD40 agonist and is added at a final concentration of 5 μg/ml. Those skilled in the art will appreciate that the amount of CD40 agonist added will depend on the agonist being used. CD40 agonism sufficiency can be monitored by assaying the B cells for overexpression of CD86 and IFN-γ receptor as well as increased expression of MHC class I and MHC class II.

Interferon gamma (IFNγ) is a soluble cytokine that is critical for innate and adaptive immunity. The IFNγ protein used with the present invention may be of any origin, but is advantageously the human IFNγ protein. In some embodiments, the IFNγ used with the invention is recombinantly expressed (e.g., in E. coli or a human cell line) and purified in house. In other embodiments, the IFNγ is purchased from a commercial source, e.g., Peprotech. The IFN-γ may be added to the B cells at the same time as the CD40 agonsit or after the CD40 agonist. In one embodiment, the IFN-γ is added at least 12 hours after the addition of the CD40 agonist to the cells In another embodiment the IFN-γ is added 18-24 hours after the addition of the CD40 agonist.

In the present methods, the B cells are also contacted with tumor-derived antigens. The term “antigen” refers to any molecule that is recognized by the immune system and that can stimulate an immune response. A “tumor-derived antigen” is an antigen that is preferentially or differentially expressed by a tumor cell and not expressed or differentially expressed on normal, healthy cells. Thus, by incubating the B cells with tumor-derived antigens, the B cells are sctivated to act as more effective antigen presenting cells and stimulate CD8+ T cells to recognize these antigens. These B cells may also be able to produce tumor-specific antibodies that may recognize the tumor. Any sample containing cancer cells may be used as a source of tumor-derived antigens for the present invention. Suitable samples include, for example, tissue samples, tumors, tumor lysates, biopsies, and bodily fluids (e.g., blood, serum, plasma, sputum, lavage fluid, cerebrospinal fluid, urine, semen, sweat, tears, saliva). Alternatively, the sample could comprise an organoid that was generated from a cancer specimen (i.e., a “tumor organoid”). In some embodiments, the tumor-derived antigen is a tumor cell lysate. In some embodiments, the tumor-derived antigen may be a single or small number of polypeptides identified as comprising tumor antigens. In certain embodiments, the tumor cell lysate is derived from a subject with cancer.

In some embodiments, the IFN-γ is added a concentration of 10 U/ml. A range from 5 U/ml to 1000 U/ml of IFN-γ can be used. The IFN-γ may be added up to 48 hours prior to contacting with the tumor-derived antigen. In certain embodiments, the IFN-γ is added for at least 12 hours and suitably at least 18-24 hours prior to contacting with the tumor-derived antigen. The incubation can be as long as 48 hours.

In the Examples, the inventors incubate the B_{Vax} with B-cell activating factor (BAFF) to enhance their survival. BAFF is a cytokine that is known to act as a potent B cell activator. Thus, in some embodiments, the B cells are also incubated with BAFF in step (b). The BAFF used with the present invention may be produced recombinantly in house or may be purchased from a commercial source, e.g., R&D Systems. In the Examples, 200 nM of BAFF was used throughout the entire ex vivo process to maintain B cell viability. Those skilled in the art will appreciate that this concentration may be varied.

The methods of the present invention involve activating 4-1BBL+ B cells by incubating them with (1) a CD40 agonist and optionally with BAFF for about 24 hours, (2) followed by addition of IFN-γ for about 24 hours, (3) and finally addition of tumor-derived antigens.

### Compositions

The present invention also provides compositions comprising 4-1BBL+ B cells made by the methods disclosed herein. In some embodiments, the compositions comprise 4-1BBL+ B cells activated in vitro with a CD40 agonist and IFN-γ for at least 20 hours and pulsed with tumor-derived antigen. In certain embodiments, the CD40 agonist is a CD40 antibody. In some embodiments, the tumor-derived antigen is a tumor cell lysate. Suitably, the composition is substantially free of cells that are not 4-1BBL+ B cells. The 4-IBBL+ cells may be obtained from blood and isolated using procedures known to those of skill in the art. For example, blood may be obtained from a subject and lymphocytes isolated. The 4-IBBL+ population of B cells can then be isolated using an antibody to 4-IBBL and isolating using FACS if the antibody is fluorescently labeled or a bead-based sorting method using an antibody labeled with a tag capable of binding to a ligand on the beads, such as via a biotin-avidin interaction. Methods of using labeled antibodies specific for cell-surface proteins to bind to and isolate the targeted cells are well known in the art.

The compositions may further comprise at least one pharmaceutically acceptable carrier. The term “pharmaceutically acceptable carrier” refers to any carrier, diluent or excipient that is compatible with the other ingredients of the formulation and not deleterious to the recipient. A pharmaceutically acceptable carrier is any carrier suitable for in vivo administration. Examples of pharmaceutically acceptable carriers suitable for use in the composition include, but are not limited to, water, buffered solutions, glucose solutions, oil-based or bacterial culture fluids. Additional components of the compositions may suitably include, for example, excipients such as stabilizers, preservatives, diluents, emulsifiers and lubricants. Examples of pharmaceutically acceptable carriers or diluents include stabilizers such as carbohydrates (e.g., sorbitol, mannitol, starch, sucrose, glucose, dextran), proteins such as albumin or casein, protein-containing agents such as bovine serum or skimmed milk and buffers (e.g., phosphate buffer).

### Methods of Treatment

Additionally, the present invention provides methods of using the B cell compositions disclosed herein to treat a subject with cancer. The methods comprise administering an effective amount of the composition to the subject. The B cells compositions may be co-admininstered with other cancer treatments including radiation, other chemotherapeutics, or other lymphocytes, including T cells or NK cells. Co-administration is used to indicate that the same subject may received an additional therapeutic in addition to the B cells compositions described here. The therapies may be admininstered to the subject simultaneously as separate treatments, as part of a unitary composition or in any order. The administration of the therapies may be administered such that one is administered before the other with a difference in administration time of 1 hour, 2 hours, 4 hours, 8 hours, 12 hours, 16 hours, 20 hours, 1 day, 2 days, 4 days, 7 days, 2 weeks, 4 weeks or more.

The methods disclosed herein can further include a conventional cancer treatment regimen. In the Examples, the inventors demonstrated that treating mice with radiation (i.e., either whole body irradiation or whole brain irradiation) and temozolomide (i.e., the current standard-of-care for the treatment of GMB) resulted in increased serum BAFF levels. As discussed above, BAFF promotes B-cell fitness and survival. Thus, in some embodiments, the methods further comprise administering radiation therapy to the subject. As used herein, the term “radiation therapy” refers to any manner of treatment of solid tumors and cancers with ionizing radiation and includes, without limitation, external beam radiotherapy, stereotatic radiotherapy, virtual simulation, 3-dimensional conformal radiotherapy, intensity-modulated radiotherapy, ionizing particle therapy, and radioisotope therapy. The enhanced, systemic production of BAFF following radiation appears to create an improved in vivo environment for adaptation of B cells. Thus, in some embodiments, the radiation therapy is administered prior to administration of the B cell composition.

In some embodiments, the methods further comprise administering a chemotherapeutic to the subject. Suitable chemotherapeutics for use with the present methods include, without limitation, platinum-based agents, such as cisplatin, gemcitabine, and carboplatin; nitrogen mustard alkylating agents; nitrosourea alkylating agents, such as carmustine (BCNU) and other alkylating agents; antimetabolites, such as methotrexate; purine analog antimetabolites; pyrimidine analog antimetabolites, such as fluorouracil (5-FU) and gemcitabine; hormonal antineoplastics, such as goserelin, leuprolide, and tamoxifen; natural antineoplastics, such as taxanes (e.g., docetaxel and paclitaxel), aldesleukin, interleukin-2, etoposide (VP-16), interferon alpha., and tretinoin (ATRA); antibiotic natural antineoplastics, such as bleomycin, dactinomycin, daunorubicin, doxorubicin, and mitomycin; and vinca alkaloid natural antineoplastics, such as vinblastine and vincristine. In some embodiments, the chemotherapeutic is a triazene such as dacarbazine, mitozolomide or temozolomide. In some embodiments, the chemotherapeutic agent is temozolomide.

Additionally, the inventors demonstrated that co-administering B_{Vax} with an anti-PD-L1 treatment resulted in enhanced CD8+ T cell activation. PD-L1 (programmed death-ligand 1) is a transmembrane protein that plays a major role in suppressing the adaptive arm of immune system. PD-L1 is considered a “checkpoint protein,” i.e., a protein that dampens the activation of an immune response, and its expression is thought to allow cancer cells to evade the host immune system. Accordingly, in some embodiments, the methods further comprise administering a checkpoint inhibitor. As used herein, the term “checkpoint inhibitor” refers to a molecule that blocks or inhibits immunosuppressive activities of checkpoint proteins. In certain embodiments, the checkpoint inhibitor is an antibody that binds PD-L1 or PD-1. Checkpoint inhibitors that comprise anti-PDI antibodies or anti-PDL 1-antibodies or fragments thereof are known to those skilled in the art, and include, but are not limited to, cemiplimab, nivolumab, pembrolizumab, MEDI0680 (AMP-514), spartalizumab, camrelizumab, sintilimab, toripalimab, dostarlimab, and AMP-224. Checkpoint inhibitors that comprise anti-PD-L1 antibodies known to those skilled in the art include, but are not limited to, atezolizumab, avelumab, durvalumab, and KN035. The antibody may comprise a monoclonal antibody (mAb), chimeric antibody, antibody fragment, single chain, or other antibody variant construct, as known to those skilled in the art. PD-1 inhibitors may include, but are not limited to, for example, PD-1 and PD-L1 antibodies or fragments thereof, including, nivolumab, an anti-PD-1 antibody, available from Bristol-Myers Squibb Co and described in U.S. Pat. Nos. 7,595,048, 8,728,474, 9,073,994, 9,067,999, 8,008,449 and 8,779,105; pembrolizumab, and anti-PD-1 antibody, available from Merck and Co and described in U.S. Pat. Nos. 8,952,136, 83,545,509, 8,900,587 and EP2170959; atezolizumab is an anti-PD-L1 available from Genentech, Inc. (Roche) and described in US Patent No. 8217149; avelumab (Bavencio, Pfizer, formulation described in PCT Publ. WO2017097407), durvalumab (Imfinzi, Medimmune/AstraZeneca, WO2011066389), cemiplimab (Libtayo, Regeneron Pharmaceuticals Inc., Sanofi, see, e.g., U.S. Pat. Nos. 9,938,345 and 9,987,500), spartalizumab (PDR001, Novartis), camrelizumab (AiRuiKa, Hengrui Medicine Co.), sintillimab (Tyvyt, Innovent Biologics/Eli Lilly), KN035 (Envafolimab, Tracon Pharmaceuticals, see, e.g., WO2017020801A1); tislelizumab available from BeiGene and described in U.S. Pat. No. 8,735,553; among others and the like. Other PD-1 and PD-L1 antibodies that are in development may also be used in the practice of the present invention, including, for example, PD-1 inhibitors including toripalimab (JS-001, Shanghai Junshi Biosciences), dostarlimab (GlaxoSmithKline), INCMGA00012 (Incyte, MarcoGenics), AMP-224 (AstraZeneca/MedImmune and GlaxoSmithKline), AMP-514 (AstraZeneca), and PD-L1 inhibitors including AUNP12 (Aurigene and Laboratoires), CA-170 (Aurigen/Curis), and BMS-986189 (Bristol-Myers Squibb), among others (the references citations regarding the antibodies noted above are incorporated by reference in their entirities with respect to the antibodies, their structure and sequences). Fragments of PD-1 or PD-L1 antibodies include those fragments of the antibodies that retain their function in binding and inhibiting PD-1 or PD-L1 as known in the art, for example, as described in AU2008266951 and Nigam et al. “Development of high affinity engineered antibody fragments targeting PD-L1 for immunoPED,” J Nucl Med May 1, 2018 vol. 59 no. supplement 1 1101, the contents of which are incorporated by reference in their entireties.

In the Examples, the inventors demonstrated that B_{Vax} can be co-administered with other lymphocytes including T cells, in particular CD8+ T cells or NK cells. Thus, in some embodiments, the methods further comprise administering T cells to the subject. The T cells used in these methods may be obtained from a subject, and then expanded and activated ex vivo via incubation with B_{Vax} to enhance their immunostimulatory capabilities. In certain embodiments, the T cells are CD8+ T cells. The T cells may be selected or engineered with antigen receptors directed against tumor antigens. As used herein, the term “chimeric antigen receptor T cell” refers to a genetically engineered antibody-T cell chimera that comprises a chimeric antigen receptor (CAR). Techniques for chimeric antigen receptor T cell therapies are known and available in the art. See, e.g., Kenderian et al., Cancer Res. 74(22):6383-9 (2014).

The terms “subject” and “patient” are used interchangeably and refer to any animal (e.g., a mammal), including, but not limited to, humans, non-human primates, rodents, and the like, which is to be the recipient of a particular treatment. For example, a suitable subject includes a subject in need of cancer treatment.

As used herein, “treating” or “treatment” describes the management and care of a subject for the purpose of combating a disease, condition, or disorder. Treating includes the administration of a composition of present invention to prevent the onset of the symptoms or complications, to alleviate the symptoms or complications, or to eliminate the disease, condition, or disorder. Specifically, compositions disclosed herein can be used to treat a cancer. Treating cancer includes, but is not limited to, reducing the number of cancer cells or the size of a tumor in the subject, reducing progression of a cancer to a more aggressive form, reducing proliferation of cancer cells or reducing the speed of tumor growth, killing of cancer cells, reducing metastasis of cancer cells or reducing the likelihood of recurrence of a cancer in a subject. Treating a subject as used herein refers to any type of treatment that imparts a benefit to a subject afflicted with a disease or at risk of developing the disease, including improvement in the condition of the subject (e.g., in one or more symptoms), delay in the progression of the disease, delay the onset of symptoms or slow the progression of symptoms, etc.

As used herein, the term “administering” refers to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, intradermal administration, intrathecal administration, intra-lymphatic and subcutaneous administration. Administration can be continuous or intermittent. In some embodiments, the composition is administered intravenously or intracranially.

The term “effective amount” refers to an amount sufficient to effect beneficial or desirable biological or clinical results. That result can be reducing, alleviating, inhibiting or preventing one or more symptoms of a disease or condition, reducing, inhibiting or preventing the growth of cancer cells, reducing, inhibiting or preventing metastasis of the cancer cells or invasiveness of the cancer cells or metastasis, or reducing, alleviating, inhibiting or preventing one or more symptoms of the cancer or metastasis thereof, or any other desired alteration of a biological system. In some embodiments, the effective amount is an amount suitable to provide the desired effect, e.g., produce an anti-tumor response. Methods for determining an effective means of administration and dosage are well known to those of skill in the art and will vary with the formulation used for therapy, the purpose of the therapy, the target cell being treated, and the subject being treated. Single or multiple administrations can be carried out with the dose level and pattern being selected by the treating physician.

The term “CD8+ T cell response” refers to the activation of CD8+ cells, enabling them to kill cells expressing the antigen to which the T cell was activated. CD8+ T cells may kill the cells through several different mechanisms, including, the secretion of cytokines (e.g., TNF-α and IFN-γ) which have anti-tumor and anti-viral microbial effects, the production and release of cytotoxic granules (e.g., perforin, and granzymes), and/or destruction of infected cells is via Fas/FasL interactions.

In the Examples, the inventors describe their 4-1BBL+ B cells as a vaccine. The term “vaccine,” as used herein, refers to a biological preparation that contains antigen or immunogen that can elicit an immune response. The antigen or immunogen can be, for example, an infectious agent, a molecule that resembles a disease-causing microorganism or cell, or a protein associated with an abnormal or diseased cell (e.g., a tumor associated antigen). For example, antigens or immunogens may be made from an attenuated or inactivated form of said microorganism or cell or its toxins. A vaccine is administered to an individual in order to stimulate that individual's immune response to said antigen or immunogen. In preferred embodiments, the antigen is a tumor-derived antigen.

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

The following examples are meant only to be illustrative and are not meant as limitations on the scope of the invention or of the appended claims.

### EXAMPLES

### Example 1—B Cell Based Immunotherapy for the Treatment of Glioblastoma

Immunotherapy has revolutionized the treatment of many tumors. However, most glioblastoma (GBM) patients have not, so far, benefited from such successes. With the goal of exploring ways to boost anti-GBM immunity, the inventors developed a B-cell-based vaccine (B_{Vax}) that consists of 4-1BBL+ B cells activated with CD40 agonism and IFNγ stimulation. B_{Vax} migrate to key secondary lymphoid organs and are proficient at antigen cross-presentation, which promotes both the survival and functionality of CD8+ T cells. A combination of radiation, B_{Vax}, and PD-L1 blockade conferred tumor eradication in 80% of treated tumor-bearing animals. This treatment elicited immunologic memory that prevented the growth of new tumors upon subsequent re-injection in cured mice. GBM patient-derived B_{Vax} were successful in activating autologous CD8+ T cells; these T cells showed a strong ability to kill autologous glioma cells. This disclosure provides an efficient alternative to current immunotherapeutic approaches that can be readily translated to the clinic.

### Materials and Methods

Human samples. All human samples (tumor, peripheral blood, and frozen tissue) were collected by the Nervous System Tumor Bank at Northwestern University (NSTB) under the institutional review board (IRB) protocol No STU00202003. All patients signed written consent. Only samples from GBM patients with greater than 50% tumor cellularity, as determined by H&E examination, were included in the study. The study was conducted following the U.S. Common Rule of ethical standards.

Mice. C57BL/6, CD45.1 C57BL/6, B-cell-deficient (μMT, BKO), Rag1 deficient (Rag1 KO) and OT-I mice were all purchased from The Jackson Laboratory. 4-1BBL deficient (4-1BBL KO) mice were obtained from Amgen. Animals were 6 to 8 weeks old at the time of the experiment initiation. All animal experimentation protocols are approved by the Institutional Animal Care and Use Committee (IACUC) under protocol #IS00002459 at Northwestern University. All animals were housed in a specific-pathogen-free (SPF) animal.

Cell lines. GL261 cells were obtained from the National Cancer Institute (NCI). GL261 cells expressing ovalbumin were obtained as previously reported in (Pituch et al., 2018). CT2A cells were a gift from Pr. Tom Seyfried (Boston College). The GL261 cell line identity and purity were evaluated annually using short tandem repeats (STR) profiling performed by the Northwestern University sequencing core facility. Both murine syngeneic glioma cell lines were maintained in DMEM (Corning) with 10% fetal bovine serum (FBS, HyClone), penicillin (100 U/mL), and streptomycin (100 mg/mL; Corning) and incubated at 37° in 5% CO2. All cell lines were routinely tested for Mycoplasma contamination every 2 months using the Universal Mycoplasma Detection Kit (ATCC® 30-1012K™).

Brain tumor injection. A total of 105 GL261 or CT2A cells were intracranially (i.c.) implanted as previously described (Wainwright et al., 2012a). Mice were anesthetized through intraperitoneal administration of a stock solution containing ketamine (100 mg/kg) and xylazine (10 mg/kg). The surgical site was shaved and prepared with a swab of povidone-iodine followed by 70% ethanol. The swabbing procedure was performed three times in total. An incision was made at the midline for access to the skull. A 1-mm-diameter burr hole was drilled 2 mm posterior to the coronal suture and 2 mm lateral to the sagittal suture. Mice were then placed in a stereotaxic frame, and tumor cells were injected in a total volume of 2.5 μL using a Hamilton syringe fitted with a 26-gauge blunt needle at a depth of 3 mm. The incision was then stapled closed.

Human immunophenotype analysis. Frozen PBMC samples (n=90) from newly diagnosed GBM patients were collected and analyzed for the levels of 4-1BBL expressing B cells (4-1BBL⁺CD20⁺CD19⁺) and CD69-expressing CD8⁺ T cells by flow cytometry. The following anti-human antibodies (Abs) were used (all from BioLegend): 4-1BBL PerCP-Cy5.5 (5F4), CD19 Pacific Blue (HIB19), CD20 BV510 (2H7), CD8 Alexa Fluor700 (RPA-T8), CD69 PE-Cy7 (FN50). Granzyme B APC (GB11), IFNγ AF700 (4S.B3) and TNFα FITC (1D6) were used for intracellular staining. Dead cells and debris were excluded from the analysis using the eBioscience Fixable viability dye eFluor780 (Thermo Fisher). Cells were acquired by the BD Symphony and analyzed by FlowJo software.

Murine immunophenotypic analysis. Tumor, blood and lymph nodes were processed for immunotype purposes as previously described (Lee-Chang et al., 2019a). Expression of 4-1BBL by B cells in blood, deep cervical lymph nodes (dCLN), superficial cervical lymph nodes (CLN) and tumor-bearing brains were analyzed by flow cytometry. Mouse Abs were all from BioLegend. CD45 BV510 (30F 11) and CD11b BV711 (ICRF44). 4-1BBL PerCP-Cy5.5 (5F4) and CD19 BV650 (1D3/CD19) were used to evaluate the levels of 4-1BBL expression in B cells. Dead cells and debris were excluded from the analysis using the eBioscience Fixable viability dye eFluor780 (Thermo Fisher). Cells were acquired by the BD Symphony and analyzed by FlowJo software.

CD86 upregulation using recombinant IFNγ. Human or murine B cells were isolated from PBMC or spleens (respectively) and isolated using the Human or Mouse B-cell isolation kit (StemCell Technologies). Cells were resuspended at 2×10⁶ cells/ml and incubated with 100 nM with human (Peprotech) or murine (R&D) BAFF and 10 U/ml human or murine IFNγ. CD86 expression was assessed by flow cytometry using the human anti-CD86 Pacific Blue (BU63, BioLegend) or anti-mouse CD86 AF700 (GL1) together with CD19 staining as described above.

Murine B_{Vax} generation. B_{Vax} were generated from 4-1BBL⁺ B cells from spleens and dCLN of tumor-bearing mice. Mice were challenged with 2×10⁶ tumor cells and were sacrificed 12-14 days after tumor inoculation. B cells were negatively isolated from spleens and dCLN using the EasySep™ Mouse B-Cell Isolation Kit (StemCell Technologies). 4-1BBL⁺ cells were then magnetically positively isolated using the anti-mouse 4-1BBL biotin (5F4, BioLegend) and anti-biotin MicroBeads (Miltenyi Biotec). Cells were resuspended at 2×10⁶ cells/ml of complete RPMI, supplemented with 100 nM of murine BAFF (R&D) and activated with anti-CD40 (FGK4.5, BioXCell). Twenty-four hours after 10 U/ml of murine IFNγ was added to the culture. After a total of 48 hours since the time of isolation, B_{Vax} were harvested, counted and ready for further utilization. In many experiments, B_{Vax} were concomitantly injected with CD8⁺ T cells also originated from spleens and CLNs of tumor-bearing mice, isolated using the Mouse CD8⁺ T-cell isolation kit (StemCell Technologies).

B_{Vax} APC phenotype. Murine B_{Vax} were tested for the expression of molecules associated with the APC function by flow cytometry. Cells were stained with the following anti-mouse Abs (all from BioLegend unless otherwise specified): IA^{b} PerCP-eFluor 710 (AF6-120.1), H-2K^{b} PE (AF6-88.5.5.3), CD86 AF700 (GL1) and 4-1BBL PerCP-Cy5.5 (5F4). After pulsing with 100 ng/ml SIINFEKL (Sigma Aldrich), the peptide presentation via H-2K^{b} was assessed using the anti-mouse SIINFEKL (SEQ ID NO: 1)—H-2K^{b} PE-Cy7 (eBio25-D1.16, eBioscience).

B_{Vax} APC function in vitro. To evaluate the ability of B_{Vax} to uptake whole ovalbumin (OVA), fluorescently labeled B_{Vax} with Cell Tracker® red CMPTX (Molecular Probes, Life Technologies) were incubated for 30 minutes with 15 μg/ml AF488-OVA (Molecular Probes, Life Technologies) in complete RPMI. Cells were washed 3 times and visualized with a Leica DMi8 microscope with a 40× objective. Data were processed and quantified using ImageJ. To evaluate the ability of B_{Vax} to present SIINFEKL (SEQ ID NO: 1) after whole OVA uptake, cells were incubated for 5 hours with 1 μg/ml whole ovalbumin (Invivogen). SIINFEKL (SEQ ID NO: 1) presentation by H-2K^{b} was assessed by flow cytometry as described above. To test the ability of whole OVA-pulsed B_{Vax} to activate TCR transgenic OT-I CD8⁺ T cells, splenic CD8⁺ T cells were isolated using the Mouse CD8⁺ T-cell isolation kit (StemCell Technologies). The inventors generated bone marrow-derived DC as previously described (Miska et al., 2016) and used as a positive control of cross-presentation. B_{Naive} (±IFNγ), B_{Vax} and DC pulsed with OVA protein were incubated at 1:1 ratio with CD8″T cells labeled with the fixable cell proliferation dye eFluor450 (eBioscience) and activated with anti-CD3/CD28 activating beads (Invitrogen) supplemented with recombinant IL2 (30 U/ml, Peprotech). CD8⁺ T-cell activation was assessed by cellular proliferation (eFluor450 dilution) and intracellular expression of GzmB by flow cytometry. Alternatively, SIINFEKL (SEQ ID NO: 1) peptide or CD8⁺ T-cell from WT C57BL/6 were used as the negative control. To test the involvement of Ag-presentation via the MHC class I, B_{Vax} were pretreated with 10 μg/ml H-2K^{b} blocking Ab (clone AF6-88.5.5.3, BioXCell). The Ab was added every day throughout the experiment (72 hours).

B_{Vax} APC function in vivo. Rag1 deficient mice were orthotopically injected with 2×10⁵

GL261 glioma cells overexpressing Ovalbumin (GL261-OVA). Seven days after tumor injections, mice were co-injected i.v. with both 2×10⁶ B_{Vax} cells (pulsed with whole OVA as described above) and 5×10⁶ eFluor450-labeled CD8⁺ T cells. Seven days after adoptive transfer, mice were sacrificed. Tumor-bearing brains and dCLN were processed to obtain single-cell suspension as described in (Lee-Chang et al., 2019a). eFluor450⁺ CD8⁺ T cells were analyzed by flow cytometry. Alternatively, B-cell deficient puMT mice were orthotopically challenged with 10⁵ GL261-OVA. Seven days after, mice received intravenously with 2×10⁶ whole OVA-pulsed B_{Naive}, B_{Vax} or B_{Vax} pretreated with 200 ng/ml Bordetella pertussis toxin (PTX, Gibco) for 1 hour at 37° C. Of note, B_{Vax(PTX)} were washed with PBS 3 times before injection. Seven days after B-cell adoptive transfer, mice were sacrificed and SIINFEKL-specific CD8⁺ T cells were analyzed by flow cytometry using the following anti-mouse Abs from BioLegend (unless otherwise specified): CD45 BV510 (30F 11), CD11b BV711 (ICRF44), CD8 BV605 (53-6.7), CD44 PerCP-Cy5.5 (IM7) and SIINFEKL (SEQ ID NO: 1)—H-2K^{b} PE-Cy7 (eBio25-D1.16, eBioscience). In a parallel experiment, B-cell deficient μuMT mice were challenged with 10⁵ CT2A. Seven days after, mice received intravenously with CT2A cells lysates-pulsed B_{Naive}, B_{Vax} or B_{Vax}+PTX. Seven days after B-cell adoptive transfer, mice were sacrificed and CD8⁺ T cells were analyzed for the intracellular expression of GzmB and IFNγ using the following anti-mouse Abs, all from BioLegend: CD45 BV510 (30F 11), CD11b BV711 (ICRF44), CD8 BV605 (53-6.7), GzmB AF647 (GB11) and IFNγ AF700 (XMG1.2). In all the experiments, dead cells and debris were excluded from the analysis using the eBioscience Fixable viability dye eFluor780 (Thermo Fisher). Cells were acquired by the BD Symphony and analyzed by FlowJo software.

In vivo B_{Vax} tracking. B-cell deficient puMT mice were challenged with 10⁵ CT2A. Nine days after, mice received intravenously with 5×10⁵ B_{Naive}, B_{Vax} or B_{Vax}+PTX labeled beforehand with the Cell Tracker® red CMPTX (Molecular Probes, Life Technologies). Three days after, mice were sacrificed and tumor-bearing brains, blood, and dCLN were analyzed for the presence of CellTracker⁺ CD19⁺ B cells by flow cytometry as described above. Alternatively, tumor-bearing Rag1 deficient mice received intravenously and concomitantly both Cell Tracker® red CMPTX B_{Vax} cells and CellTracker Green CMFDA-labeled CD8⁺ T cells. Three days after the cell adoptive transfer, mice were sacrificed and spleens were collected. Tissue samples were embedded in OCT (Thermo Fischer) and flash-freeze. Sections (6 μm) were obtained and the presence of B_{Vax} (red cells) and CD8⁺ T cells (green) were analyzed by fluorescent microscopy (Leica DMi8). Data were processed and quantified using ImageJ.

Serum BAFF measurement by ELISA. Mice were bled retro-orbitally and samples were allowed to clot by leaving them at room temperature for 30 minutes. Clots were removed by centrifuging at 1,000-2,000×g for 10 minutes at 4° C. Sera were stocked at −80° C. until use. BAFF levels were measured using the Mouse BAFF/BlysTNFSF13B Quantikine ELISA Kit (R&D) as directed by the manufacturer.

In vivo B_{Vax} adaptation upon radiation. WT C57BL/6 mice were intracranially challenged with 10⁵ CT2A cells. After 7 days, mice received total body irradiation (9Gy) using a Gammacell 40 Exactor (Best Theratronics). Two days after, mice received intravenously 5×10⁶ B_{Vax} originated from CT2A-bearing CD45.1 mice. Seven days after cell adoptive transfer, CD45.1⁺ B_{Vax} were evaluated in the spleen, dCLN and tumor-bearing brains by flow cytometry using the anti-mouse CD45.1 eFluor450 (A20) and total CD45 PE (30-F11) from BioLegend. Alternatively, CD45.1⁺ B_{Naive} activated 4-1BBL⁻ B cells with anti-CD40 and IFNγ (B_{Act}) and B_{Vax} were measured by flow cytometry using CD45.1 eFluor450 (A20) and CD45.2 PE-Cy7 (104). A group of mice received B_{Vax} treated with 10 μg/ml BAFF receptor blocking Ab (7H22-E16, BioLegend). Mice subsequently received 100 μg of BAFF receptor blocking Ab intraperitoneally every day for 7 days. In-vivo B-cell proliferation was assessed by the expression of Ki67 (PE, BioLegend). Lymphopenia in irradiated mice was assessed by the numbers of CD45⁺ leukocytes in the blood and spleen 5 days after irradiation. In a parallel experiment, 2×10⁶ DCs and B_{Vax} labeled with the eBioscience Fixable cell proliferation dye eFluor450 (eBioscience, Thermo Fischer) was injected intravenously. A group of mice received an intradermal injection of DCs. Seven days after cell adoptive transfer, mice were sacrificed and cell proliferation was assessed by flow cytometry as the dilution of eFluor450 dye within the CD45⁺ cell population in the dCLN and spleens.

B_{Vax} therapeutic effect. C57BL/6 mice were intracranially injected with 10⁵ CT-2A cells. After 7 days, mice received total body irradiation (9Gy) using a Gammacell 40 Exactor (Best Theratronics). Two days after, mice received intravenously with 1.5×10⁶ B_{Vax}±4-5×10⁶ CD8⁺ T cells isolated and process as described above.

Alternatively, B-cell deficient mice were intracranially injected with 10⁵ CT-2A cells. After 9 days, mice received intravenously with 1.5×10⁶ B_{Naive}, B_{Act} or B_{Vax}, ±4-5×10⁶ CD8⁺ T cells isolated and process as described above. In a parallel experiment, B_{Vax} were pretreated with 10 μg/ml of 4-1BBL blocking Ab (clone TKS-1, BioXCell) before injection, and mice received 2 intraperitoneal injections of 500 μg/Kg.

In-vivo CD8⁺ T-cell tracking imaging. Spectral Lago live small animal imaging system was used to determine the in-vivo trafficking of CD8⁺ T cells to the brain of glioma-bearing mice. Seven days after tumor implantation, mice received whole-body irradiation as described above (9Gy). Forty-eight hours after each mouse received intravenously with 5×10⁶ CellTracker Deep Red-labeled CD8⁺ T cells with 1.5×10⁶ B_{Vax} (i.v) or 1.5×10⁶ DC (i.d). At scheduled time points (24, 30, 50 and 72 hours after cell adoptive transfer), the mice were anesthetized and scanned with the excitation at 640 nm and the emission at 690 nm. The fluorescence intensities in regions of interest (ROI) were calculated using the Aura Imaging Software.

Radioation, B_{Vax}, CD8⁺ T cells and anti-PD-L1 combination therapy. To evaluate the persistence and memory phenotype of adoptively transferred CD8⁺ T cells in mice receiving the combination of RT, B_{Vax}, and anti-PD-L1, WT C57BL/6 were challenged with 2×10⁵ CT2A cells intracranially and were used as donors of B_{Vax} and CD8⁺ T cells. Host mice received 10⁵ CT2A cells intracranially. Seven days after, mice received total body irradiation (9Gy, Gammacell 40 Exactor, Best Theratonics). Two days after mice received 2×10⁶ B_{Vax} pulsed with CT2A protein homogenates (generated as described above) and 3×10⁶ CellTracker PE CMTX-labeled CD8⁺ T cells were injected intravenously. Twenty-four hours later mice received intraperitoneally 500 μg/mouse of anti-PD-L1 (10F.9G2, BioXCell). Ten days after, mice were sacrificed and CellTracker⁺CD8⁺ T cells were analyzed by flow cytometry. The following anti-mouse Abs (BioLegend) were used to analyze the adoptively transferred CD8⁺ T cells: CD45 BV510 (30F 11), CD11b BV711 (ICRF44), CD8 BV605 (53-6.7), CD44 PerCP-Cy5.5 (IM7), CD62L BV421 (MEL-14), (53-6.7), CD44 PerCP-Cy5.5 (IM7).

To test the effect of RT, B_{Vax}, CD8⁺ T cells and anti-PD-L1 in animal survival, mice received total body irradiation (9Gy, Gammacell 40 Exactor, Best Theratonics) 7 days after tumor implantation. Forty-eight hours after, mice received 1.5×10⁶ B_{Vax} pulsed with CT2A protein homogenates (B_{Vax(CT2A)}) and 2×10⁶ CD8⁺ T cells intravenously. Twenty-four hours after mice received intraperitoneally with 500 μg/mouse of anti-PD-L1. After 2 days, mice received a second cycle of B_{Vax(CT2A)}+CD8⁺ T cells followed by 200 μg/mouse of anti-PD-L1. After 2 days, mice received the third cycle of B_{Vax(CT2A)}+CD8⁺ T cells followed by 200 μg/mouse of anti-PD-L1. Seventy-five days after tumor injection, surviving mice were re-challenged in the opposite hemisphere (left) to the initial injection site with 10⁵ CT2A cells intracranially.

Histopathological and immunophenotype analysis of long-term survivors' brains. Long-term survivors (LTS) were monitored daily. Mice were sacrificed 245 days after initial tumor injection. Three brains were formalin-fixed for 24 hours at room temperature. The injection needle track was identified and sagittal sectioning was performed for every mouse brain. For LTS, brains were cut in the right (LTS-R) and left (LTS-L) hemispheres. Tissue samples were paraffin-embedded for immunohistochemistry evaluation. Age-matched (10 months old) control mice were sacrificed after 14 days after CT2A tumor injection (10⁵ cells/mouse intracranially). Tumor burden was analyzed by H&E staining. To evaluate the CD8⁺ T-cell infiltration, sections were stained with anti-mouse CD8 (clone 4SM16, eBioscience, 1/100 dilution) and Donkey anti-Rat IgG (Jackson ImmunoResearch). The histopathological processing of the samples was performed at the Mouse Histology and Phenotyping Laboratory (MHPL) at Northwestern University. Five brains were used for the immune cell's phenotype. For immunophenotype analysis of brain-infiltrating immune cells, brain and peripheral tissue samples were processed as previously described in (Lee-Chang et al., 2019a). Cells were stained for the following anti-mouse Abs (from BioLegend): CD45 BV510 (30F 11), CD11b BV711 (ICRF44), CD4 PE-Cy7 or AF700 (GK1.5), CD8b FITC (YTS156.7.7) or CD8 BV605 (53-6.7), CD44 PerCP-Cy5.5 (IM7), CD44 PerCP-Cy5.5 (IM7), KLRG1 APC (2F1/KLRG1), TIGIT PE-Cy7 (1G9), PD-1 FITC (29F.1A12), Foxp3 BV421 (FJK-16S), CD19 PE or BV605 (1D5), 4-1BBL PerCP-Cy5.5 (TKS-1) and LAP/TGFB PE (TW7-16B4).

Whole brain radiation, temozolomide and B_{Vax}, CD8⁺ T cells and anti-PD-L1 combination therapy. Alternatively, C57BL/6 mice were intracranially injected with 10⁵ CT-2A cells. After 7 days, mice's brains were irradiated with a total of 9Gy (fractionated 3 times 3Gy, Gammacell 40 Exactor, Best Theratonics). At day 11 post-tumor injection mice received intraperitoneally 50 mg/kg of TMZ for 5 consecutive days. Twenty-four hours after the last TMZ dose, mice received 1.5×10⁶ B_{Vax}±4-5×10⁶ CD8⁺ T cells isolated and process as described above.

Patient-derived B_{Vax} generation and autologous CD8⁺ T-cell activation assay. GBM patients' peripheral blood samples were collected in EDTA-treated tubes and PBMC were isolated by Ficoll gradient (GE Healthcare). PBMC B cells were obtained using the EasySep™ Human B-cell isolation Kit II (StemCell Technologies). 4-1BBL-expressing B cells were then magnetically isolated using the human anti-4-1BBL biotin (BioLegend) and then anti-biotin MicroBeads (Miltenyi Biotec). 4-1BBL-expressing B cells were resuspended at 2×10⁶ cells/ml in complete RPMI and stimulated with 5 μg/ml human anti-CD40 (clone, FGK4.5, BioXCell). After 24 hours, 10 U/ml of recombinant human IFNγ (Peprotech) was added. Cells were incubated for additional 24-48 hours. B cells were supplemented with 100 nM of recombinant human BAFF

(Peprotech) throughout the entire in-vitro activation process. T cells were isolated using the Easy Sep™ Human T-Cell Isolation Kit (StemCell Technologies) and labeled with 10 μM of the eBioscience™ cell proliferation dye eFluor 450 (Thermo Fischer). Cells were activated with T-cell activator anti-CD3/CD28 beads (Dynabeads, Invitrogen, Thermo Fischer) at 1:3 beads:T-cell ratio supplemented with IL2 (50 U/mL; Peprotech) and cocultured at a 1:1 ratio with tumor-infiltrating or PBMC CD19⁺ B cells for 72 hours. CD8⁺ T-cell proliferation (eFluor450 dilution) and activation status (intracellular GzmB and IFNγ expression) were analyzed using flow cytometry.

Human B_{Vax}-mediated autologous CD8⁺ T-cell activation and tumor cell killing assays. Freshly resected tumor samples were diced using a razor blade and incubate for 30 minutes at 37° C. in a tissue culture dish (100 mm diameter) with digestion buffer, consisting of 4 mL of Hank's balanced salt solution (HBSS, Gibco) supplemented with 8 mg of collagenase D (Sigma-Aldrich), 80 μg DNase I (Sigma-Aldrich), and 40 μg TLCK (Sigma-Aldrich) per approximately 2 grams of the tumor sample. The sample was mixed by pipetting up and down several times every 10 minutes. Then, the cell suspension was mechanically dissociated using a tissue homogenizer (Potter-Elvehjem PTFE pestle) in HBSS. Cells were cultured ex-vivo as tumor spheroids in Neurobasal Medium with 1% B27 supplement, 0.25% N2 supplement, 1% Penicillin Streptomycin, 1 μl g/ml heparin, 20 ng/ml human bFGF, and 20 ng/ml human EGF. Peripheral blood was processed by density gradient separation (Ficoll, GE Healthcare) to obtain peripheral blood mononuclear cells.

Tumor lysate preparation: Tumor single-cell suspension was resuspended at 10⁶ cells/ml of PBS and underwent 5 freeze-thaw cycles and 1 minute of sonication.

B_{Vax} generation: B cells were isolated using the Human B-cell isolation kit (StemCell Technologies). 4-1BBL⁺ B cells were magnetically isolated using anti-human 4-1BBL biotin (clone 5F4, BioLegend) and anti-biotin Microbeads (Miltenyi Biotec). Cells were cultured at 2×10⁶ cells/ml of cRPMI supplemented with with 5 μg/ml anti-CD40 (clone 5C3, BioLegend) and 100 nM of recombinant human BAFF (R&D). Twenty-four hours after, 1000 U/ml of recombinant IFNγ (Peprotech) was added to the culture for additional 18-20 hours. B_{Vax} were pulsed with tumor lysates for 5 hours at 37° C. Cells were washed twice with cRPMI.

CD8⁺ T-cell activation assay: After PBMC isolation, CD8⁺ T cells were isolated using the Human CD8⁺ T-cell isolation kit (StemCell Technologies) and cultured with 30 U/ml of recombinant IL2 (Peprotech) for 24 hours, until B_{Vax} were generated and pulsed with tumor lysates. CD8⁺ T cells were labeled with the eBioscience Fixable cell proliferation dye eFluor450 (eBioscience, Thermo Fischer) and mixed with B_{Vax} at a 1:1 ratio supplemented with 30 U/ml of recombinant IL2. CD8⁺ T-cell activation was assessed by flow cytometry as cell proliferation (dilution of eFluor450 dye) and expression of intracellular GzmB.

Tumor cell cytotoxicity assay: To assess activated CD8⁺ T-cell cytotoxic capabilities after coculture with B_{Vax}, CD8⁺ T cells were magnetically isolated using anti-CD8 biotin (clone SK1, BioLegend) and anti-biotin Microbeads (Miltenyi Biotec). Of note, magnetic isolation was performed in both control (no B_{Vax}) and B_{Vax} treated groups. Isolated CD8⁺ T cells were added at various effector:target ratios with labeled ex-vivo tumor cells and cytotoxicity was assayed using IncuCyte S3 Live Cell Analysis System (Sartorius, Essen BioScience). Tumor cells were pre-labeled with CFSE according to the manufacturer's protocol, and cultured in 96 well plates with 250 nM IncyCyte Cytotox Red Reagent with or without the addition of effector cells at 20:1, 10:1, 5:1, and 2.5:1 effector:target cell ratios. Assay controls to account for spontaneous target cell death (target cells alone) and maximum cell death (target cells+0.1% Triton X) were included to allow for quantification of cell killing. Live cell images (4 per well, with 10× objective) were taken at 30 minutes intervals from hour 0-hour 2.5, and 1-hour intervals thereafter until completion of the assay at 12.5 hours after plating. For image analysis 5% green fluorescence was removed from the red channel, and quantification was performed at each experimental condition as a ratio of the [total red count] to [total green cell count]. The total red count is indicative of cell death, and the total green cell count is indicative of viable cells and cell proliferation over the course of the assay. To calculate Percent Killing of each condition (relative to the scale set by spontaneous and maximum cell death controls) the following calculation was performed at each condition: % Killing=(% Experimental−% Spontaneous)/(% Maximum Release−% Spontaneous)×100. IncuCyte Live Cell Analysis was performed in the Analytical bioNanoTechnology Core Facility of the Simpson Querry Institute at Northwestern University. ANTEC is currently supported by the Soft and Hybrid Nanotechnology Experimental (SHyNE) Resource (NSFECCS-1542205).

B_{Vax}-derived Ig ELISA. B-cell subsets-derived Ig subtypes were measured using the Ig Isotyping Mouse Instant ELISA™ Kit (Invitrogen, Thermo Fischer).

Brain sections IF using B_{Vax}-derived Ig. CT2A-bearing B-cell deficient mice were sacrificed 14 days after tumor injection. Mice were perfused with chilled PBS and brains were collected and freeze in OCT (Thermo Fischer). Brain tissue sections were fixed with pre-chilled methanol for 10 minutes at −20° C. Sections were washed and rehydrate for 20 minutes at room temperature with PBS, and blocked with 1% BSA in PBS+5% Donkey serum (NDS), 1 hour at room temperature. Mice serum were diluted at 1:10 in 0.1% BSA in PBS and applied on the section without washing the blocking buffer. Sections were incubated for additional 2 hours at room temperature, washed 3 times with PBS and stained with secondary anti-mouse IgG Cy5 (Jackson ImmunoResearch) diluted 1:500 in 0.1% BSA in PBS 45 minutes at room temperature. Slides were washed 3 times with PBS and mounted with Fluoroshield™ with DAPI (Sigma-Aldrich).

B_{Vax}-derived Ig purification. Mice serum IgG were isolated using the NAb™ Protein A/G Spin Column (Thermo Scientific). Eluted proteins were concentrated using the Amicon Ultra 15 ml 30K (Millipore Sigma). Final IgG concentration was measure by Bradford method.

SIINFEKL (SEQ ID NO: 1) -reactive B_{Vax} Ig ELISA. ELISA 96-well microplate (Corning®, Sigma-Aldrich, USA) were coated overnight at 4° C. with either 10 μg/ml of SIINFEKL (SEQ ID NO: 1) (Sigma-Aldrich, USA) diluted in PBS. Plates were washed three times with PBS containing 0.05% tween-20 to remove unbound SIINFEKL (SEQ ID NO: 1). A volume of 100 μl of PBS-BSA 1% was used for blocking for 1 hour at room temperature. Fifty microliters per well of diluted IgG sample were added to corresponding wells in duplicate. The plates were incubated at room temperature for 1 hour and washed three times. To measure ovalbumin-specific antibody, anti-mouse IgG coupled to peroxidase was added to wells at the dilution of 1:1,000 (Thermo Fischer). Secondary antibodies were incubated for 1 hour at room temperature. Plates were washed three times and 50 μl of streptavidin coupled with horse-peroxidase was added to the plates, and incubated at room temperature for 20 min. After three washes, the signal was revealed by adding 50 μl of tetramethylbenzidine (TMB) and the plates were incubated at room temperature for 15 minutes in the dark. The reaction was stopped by adding 25 μl of H2SO4 2N. Optical density at 492 nm was measured using the Genemate Uniread 800 plate reader.

B_{Vax}-derived Ig therapeutic effect. B-cell deficient mice received intracranially 10⁵ CT2A cells intracranially using a cannula system. Briefly, mice were anesthetized and a skin incision ˜10 mm in length was made over the middle frontal to the parietal bone to expose the surface of the skull. A 26-gauge sterile guide cannula for mice (Plastics One) was installed into the mouse brain at 2 mm depth through the burr hole generated during tumor implantation as described above. Tissue glue was applied around the burr hole and secure the protrusion of the cannula for long term stable positioning. The scalp was closed with surgical glue around the implantation site. A protection dummy cannula was used to secure the protrusion end during the post-op recovery and following observation period. For anti-CD20 injection, a 33-gauge sterile syringe was inserted into the guide cannula. The syringe can be covered with a sleeve designed to extend 1 mm beyond the tip of the guide cannula. Purified serum IgG (12.5 μg/mouse/injection) was injected into the brain (final volume of 2.5 μL/injection). After injection, the cannula was covered using a 33-gauge dummy cannula for mice.

Statistical analysis. Data are shown as mean±SD for a continuous variable and number (percentage) for a categorical variable. Differences between two groups were analyzed by Student's t-test or Wilcoxon Rank-sum test as appropriate. Differences among multiple groups were evaluated using one-way ANOVA with post hoc Tukey's test, or Kruskal-Wallis H Tests followed by post hoc Dunn's multiple tests as appropriate. Survival curves were generated via the Kaplan-Meier method and compared by log-rank test and multiple comparisons were adjusted using the Bonferroni method. Categorical variables were analyzed using Fisher's exact tests or Chi-square tests as appropriate. All the tests are two-sided and p-values or Benjamini-Hochberg adjusted false discovery rates less than 0.05 were considered as significant. Statistical analyses were performed using SAS9.4 and GraphPad Prism7.03.

### Results

### Functional Status of 4-1BBL-Expressing B Cells

In GBM patients' peripheral blood, 4-1BBL⁺ B cells represented 13.75±2.3% of total CD45⁺ compartment (PBMC, n=90, Table 1 and FIG. 1A). These 4-1BBL⁺ B cells show increased levels of intracellular TNFα and IFNγ, as well as expression of activation markers CD86 and CD69 expression levels (FIG. 1B) compared to 4-1BBL⁻ B cells, suggestive of their activated status. The relative abundance of 4-1BBL⁺ B cells was associated with increased numbers of activated CD69 CD8⁺ T cells (FIG. 1C), suggestive of a systemic activated immune-profile of the patients. Accordingly, GBM patient-derived 4-1BBL⁺ B cells show greater ability to enhance CD8⁺T-cell co-stimulation in the presence of exogenous TCR stimulation (anti-CD3) compared to 4-1BBL⁻ B cells, as shown by increased cell proliferation (measured as expansion index, FIG. 1D) and expansion of effector IFNγ and GzmB-expressing cells (FIG. 1E). In the preclinical CT2A glioma mouse model, 4-1BBL expression on B cells was not detected in tumor-bearing brains. Its expression was significantly higher in B cells from both deep and superficial cervical lymph nodes (CLN) after tumor inoculation, suggesting a tumor-mediated induction of 4-1BBL expression by B cells (FIG. 9A). The ability of B cells to co-stimulate CD8⁺⁰ T cells largely depended on the expression of 4-1BBL. 4-1BBL up-regulation in B cells is driven after B-cell receptor (BCR) stimulation and CD40 cognate help (Futagawa et al., 2002). In support of these observations, BCR and CD40 stimulated B cells, in the presence of BAFF could promote the proliferation of effector CD8⁺ T cells (FIG. 9B). This function required the ability of these B cells to express 4-1BBL, as its absence dampened the CD8⁺ T-cell activation function (FIG. 9B). Overall, these data suggest that 4-1BBL⁺ B cells are activated cells, capable of expanding and promoting the CD8⁺ T cell effector phenotype.

TABLE 1
Baseline characteristics of newly diagnosed GBM patients.

| Patient Characteristics | N = 90 | |
|---|---|---|
| Age at diagnosis (years) (IQR) | 64 | (55-70) |
| Gender | | |
| Male (%) | 47 | (52%) |
| Female (%) | 43 | (48%) |
| Race | | |
| White (%) | 75 | (97%) |
| Black (%) | 2 | (3%) |
| Ethnicity | | |
| Hispanic (%) | 75 | (97%) |
| Non-hispanic (%) | 2 | (3%) |
| IDH-1 Status | | |
| Wild-type (%) | 84 | (93%) |
| Mutated (%) | 6 | (7%) |
| MGMT promoter | | |
| Unmethylated (%) | 53 | (59%) |
| Methylated (%) | 34 | (38%) |
| P53 stain (%) (IQR) | 5% | (2-20%) |
| Ki 67 (%) (IQR) | 30% | (16-40%) |
| Preop steroid use | 85 | (94%) |
| Median overall survival (months) | 15 | (12-19) |
| (95% CI) | | |
| 1-year survival # at risk | 37 | (59%) |
| (Survival Probability) | | |
| 2-year survival # at risk | 12 | (26%) |
| (Survival Probability) | | |

Abbreviations: Interquartile range: IQR.

### CD40 and IFNγR Stimulation Potentiate the APC Phenotype and Function of 4-1BBL⁺ B Cells.

CD40 ligation is a well-studied process that leads to B-cell activation, proliferation, and enhancement of Ag-presenting and co-stimulatory functions (Ahmadi et al., 2008; Lapointe et al., 2003) (FIG. 9B). However, it has also been associated with the generation of immunosuppressive and regulatory B cells in different inflammatory and autoimmune conditions (Yoshizaki et al., 2012). To generate stable APC B cells, the inventors tested the activation of the IFNγR, as it can drive B-cell co-stimulatory molecules expression (Braun et al., 2002). The inventors observed that IFNγ caused up-regulation of CD86 expression on unstimulated human B cells (FIG. 10A), a key co-stimulatory molecule in CD8⁺ T-cell activation (Lee-Chang et al., 2014). The pro-activating effect of IFNγ was proven by the utilization of B cells deficient in IFNγR (FIG. 10B). However, IFNγ alone could not promote B cells able to activate CD8⁺ T cells in the presence of TCR stimulation (anti-CD3) (FIG. 9B), suggesting that IFNγR stimulation could potentiate rather than initiate the B-cell-mediated CD8⁺ T-cell co-stimulation. In addition, the inventors observed that both CD40 and IFNγR stimulation additively up-regulated CD86 (FIG. 10B), suggesting that dual activation can further promote the APC-like phenotype of B cells.

Based on these results, CD40 agonism and IFNγ were used to activate in-vitro 4-1BBL* B cells isolated from glioma-bearing mice's secondary lymphoid organs such as deep and superficial CLN and spleens (FIG. 10C). BAFF was used to enhance B-cell survival. After a total of 48 hours of culture, cells were harvested and evaluated for the expression of APC markers. Compared to 4-1BBL⁻ B cells incubated with only BAFF (designated as B_{Naive}), activated 4-1BBL⁺ B cells (designated as B_{Vax}) showed up-regulation of both major histocompatibility complex (MHC)-class I (H-2K^{b}) and II (IA^{b}) on their surface (FIG. 10C and D). After pulsing the cells with the ovalbumin (OVA) peptide SIINFEKL (SEQ ID NO: 1), B_{Vax} highly co-express SIINFEKL (SEQ ID NO: 1)—H-2K^{b} complex and co-stimulatory markers CD86 and 4-1BBL (FIG. 1F).

Professional APCs are known for their ability to cross-present exogenous antigens to CD8⁺ T cells via MHC class I (Fu and Jiang, 2018). To evaluate whether B_{Vax} can cross-present, they were incubated with a fluorescently conjugated OVA protein (AlexaFuor488-OVA, FIG. 1G). OVA protein uptake was visualized using epifluorescent microscopy (FIG. 1G). After 3 hours, the inventors observed B_{Vax} had substantial levels of surface H2K^{b}-SIINFEKL (SEQ ID NO: 1) complex, as observed by flow cytometric analysis, while B_{Vax} treated with Golgi transporter inhibitor brefeldin A (BFA) did not (FIG. 1H). Next, the inventors evaluated the ability of OVA-pulsed B_{Vax} to activate SIINFEKL (SEQ ID NO: 1)-specific OT-I CD8⁺ T cells. The inventors included bone marrow-derived DCs as a gold standard of professional APC able to cross-present. B_{Naive}, B_{Vax} and DCs pulsed with SIINFEKL (SEQ ID NO: 1) peptide induced the proliferation of OT-I CD8⁺ T cells, as well as their up-regulation of granzyme B (GzmB) (FIG. 10E). This phenomenon was dependent on the T-cell receptor (TCR)-ligation, as un-pulsed cells (No Ag) failed to activate OT-I CD8⁺ T cells (FIG. 10E) and SIINFEKL (SEQ ID NO: 1)-pulsed APCs could not activate CD8⁺ T cells from WT C57BL/6 mice (FIG. 10F). However, when B cells and DCs were pulsed with OVA protein, only B_{Vax} and DCs were able to successfully activate OT-I CD8⁺ T cells (FIG. 1I). Blockade of the MHC-class I abrogated the APC function of B_{Vax} (FIG. 1J). Altogether, these data suggest that B_{Vax} have the adequate constellation of surface molecules to promote CD8⁺ T-cell activation, and can cross-present antigens via the MHC-class I as a professional APC in-vitro.

### B_{Vax} are Potent APC's In Vivo

Next, the inventors aimed to test the APC function of B_{Vax} in vivo. First, C57BL/6 mice were intracranially injected with GL261 overexpressing the OVA protein (GL261-OVA). These mice were used as B_{Vax} and CD8⁺ T-cell donors. B_{Vax} were pulsed with OVA and CD8⁺ T cells were fluorescently labeled using the eFluor450 dye, and concomitantly injected intravenously into Rag1-deficient mice bearing GL261-OVA tumor. Administration of B_{Vax} increased the numbers of eFluor450⁺ CD8⁺ T cells in tumor-bearing brains and deep CLN when compared to B_{Naive} or untreated mock groups (FIG. 2A). Next, B-cell deficient (BKO) mice bearing GL261-OVA received OVA-pulsed B_{Vax}. Treated mice showed a substantial increase of endogenous SIINFEKL (SEQ ID NO: 1)-specific CD8⁺ T cells infiltrating the tumor-bearing brains (FIG. 2B and FIG. 10G). A group of mice that received B_{Vax} pre-treated in vitro with the pertussis toxin (PTX), which inhibits G-protein-mediated cellular migration (Cyster and Goodnow, 1995), did not induce SIINFEKL (SEQ ID NO: 1)-specific CD8⁺ T cells as well (FIG. 2B). In the CT2A glioma model, B_{Vax} pulsed with CTA-tumor lysates (B_{Vax} (CT2A)) increased the number of activated GzmB- and IFNγ-producing CD8⁺ T cells in the tumor-bearing brains (FIG. 2C). The loss of CD8⁺ T-cell activation in mice receiving B_{Vax(CT2A)}+PTX highlights the importance of the tissue recruitment of B_{Vax}. Accordingly, B_{Vax} co-localize with CD8⁺ T cells in secondary lymphoid organs such as spleens and CLNs after concomitant intravenous injection (FIG. 2D). In addition, while B_{Vax} (and B_{Naive}) were detected in the draining dCLN and the circulation, only B_{Vax} were found in the tumor-bearing brains (FIG. 11A). As the inventors have recently reported that myeloid-derived suppressive cells (MDSCs) could generate regulatory B cells (Bregs) within the tumor vicinity (Lee-Chang et al., 2019b) the inventors next determined whether tumor-infiltrating B_{Vax} could be converted into immunosuppressive B cells. The inventors' data showed that tumor-infiltrating B_{Naive} and B_{Vax} differ in their ability to promote CD8 T-cell activation. After intracranial injection, B_{Vax} maintained their CD8+ T-cell activating function whereas injection of Bₙₐᵢᵥₑ resulted in T-cell inhibition (FIG. 11B, after injection). Altogether, the results confirm that B_{Vax} are resistant to tumor immunosuppression and maintained their T-cell activating function in vivo. In support of this, B_{Vax} therapy extended CT2A-bearing animal survival when compared to mice treated with B_{Naive} or activated B-cells (B_{Act}) (Mock median survival days: 24; B_{Naive}: 17.5; B_{Act}: 18; B_{Vax}: 34; Mock vs B_{Vax}: p=0.006; FIG. 11C). B_{Vax} therapeutic effect was abrogated when 4-1BBL blocking Ab was used (Mock median survival days: 23.5; B_{Vax}: 35.5; B_{Vax}+anti-4-1BBL: 22.5; B_{Vax} : 34; B_{Vax} vs B_{Vax}+anti-4-1BBL: p=0.0016; FIG. 11D), suggesting the key role of this molecule in B_{Vax}-mediated immune functions.

### Radiotherapy Promotes B_{Vax} Expansion and Persistence in the Secondary Lymphoid Organs

It has been reported that, in GBM patients, radiation and temozolomide (TMZ) treatment increases the systemic production of BAFF, a key factor for the survival of B cells (Sanchez-Perez et al., 2013; Saraswathula et al., 2016). This suggests that current treatment might provide an adequate environment for the in vivo adaptation of B cells. To test this hypothesis, CT2A-bearing mice received a lymphodepleting dose of irradiation (RT, FIG. 12A). Then, mice received B_{Vax} obtained from CD45.1⁺ congenic mice. After 5 days, the inventors observed that CD45.1⁺ B_{Vax} counts were increased in mice treated with RT when compared to the untreated mock group (FIG. 3A). Serum BAFF levels were significantly higher in the RT group and sustained over the duration of the experiment (5 days, FIG. 3B). Since BAFF levels in tumor-bearing mice were lower than in tumor-free mice, this suggests that the tumor might disrupt the B-cell homeostatic balance and drive the drastic B-cell depletion observed in the peripheral compartment (Lee-Chang et al., 2019a). As BAFF is a survival factor for any B-cell subtype (Mackay and Browning, 2002), the inventors performed the same adoptive transfer of CD45.1⁺ B cells, but using different B-cell subsets such as B_{Naive}, activated with CD40 agonist and IFNγ B cells but 4-1BBL⁻ (B_{Act}) and B_{Vax}. All B-cell subtypes were found in the spleen, however, more B_{Vax} were obtained in the dCLN (FIG. 3C), known to drain the central nervous system (Louveau et al., 2018). The majority of dCLN-homing B_{Vax} were in a proliferative cellular state, as shown by the expression of Ki67 (FIG. 3C). Pretreatment of B cells and systemic injection with BAFF receptor (BAFF-R) blocking antibody reduced B_{Vax}'s counts in vivo (FIG. 3D), suggesting that BAFF secretion upon RT controls B_{Vax} in vivo adaptation.

Next, B_{Vax} therapeutic effect was tested in vivo in mice treated with or without RT. CT2A-bearing mice were treated with RT 7 days after tumor implantation and B_{Vax} pulsed with CT2A-tumor lysates were intravenously injected 2 days after RT, after confirming tumor engraftment and significant tumor mass by histology (FIG. 12B). B_{Vax} pulsed with tumor lysates provided slight but significantly extended animal survival in combination with RT (mock median survival days: 17; RT: 18; B_{Vax}: 22; B_{Vax}+RT: 28. B_{Vax} vs B_{Vax}+RT p<0.0001, FIG. 11C). As RT induced general lymphopenia (FIG. 12A), the inventors postulated that the lack of B_{Vax} 's target cells (CD8⁺ T cells) would limit the therapeutic effectiveness of the vaccine. Thus, the inventors administered B_{Vax} (pulsed with tumor lysates) concomitantly with CD8⁺ T cells obtained from CT2A glioma-bearing mice. Mice that received RT and a single shot of B_{Vax} showed improved overall survival (Mock vs B_{Vax} p-0.0001). However, mice that received both B_{Vax} and CD8⁺ T cells survived longer compared to all other groups (Mock median survival: 16 days; CD8⁺ T: 19 days; B_{Vax}: 27.5 days; Bᵥₐₓ+CD8⁺ T: 35 days, B_{Vax} vs B_{Vax}+CD8⁺ T p=0.0001, FIG. 3E). Similar results, although modest, were obtained in mice treated with B_{Vax} and CD8⁺ T cells at late stages of the tumor progression (15 days after tumor implantation; mock median survival days: 20; B_{Vax} (D9): 26; B_{Vax}+CD8⁺ T (D9): 35; B_{Vax} (D15): 19; B_{Vax}+CD8⁺ T(D15): 25; B_{Vax}+CD8⁺ T (D9) vs B_{Vax}+CD8 ⁺ T (D15) p=0.0003, FIG. 12D). A single shot of B_{Vax} (pulsed with tumor lysate)+CD8⁺ T-cell combination provided improved therapeutic benefit as compared to DC (pulsed with tumor lysates), that was administered either intravenously or intradermally (mock median survival days: 16; CD8⁺ T: 22; DC_{(id)}+CD8⁺ T: 16; DC₍ᵢᵥ₎+CD8⁺ T: 20; B_{Vax(iv)}+CD8⁺ T: 34; DC₍ᵢᵥ₎+CD8⁺ T vs B_{Vax(iv)}+CD8⁺ Tp 0.0029; FIG. 3F). To test whether in vivo persistence of these different cellular-based therapies was associated with different outcomes, B_{Vax} and DC were fluorescently labeled with the cell proliferation dye eFluor450 and administered to RT-treated CT2A-bearing mice. Five days after the adoptive transfer, eFluor450⁺ cells were quantified. Accumulation of B_{Vax} was significantly higher than DCs. By examining the dilution of the eFluor450 dye, the inventors observed that B_{Vax} had a high proliferative phenotype (FIG. 3G).

In addition, the inventors performed in vivo imaging tracking of CD8⁺ T cells labeled with far-red fluorescence co-injected with either B_{Vax} or DC (both pulsed with CT2A-tumor lysates). CD8⁺ T cell accumulation in the CT2A-bearing brains was enhanced when cells were administered concomitantly with B_{Vax} 30 hours after injection (FIG. 4A). The decrease in the signal at later time points could be due to the limited display of dye fluorescence. Thus, in a parallel experiment, mice received CD45.1⁺CD8⁺ T cells together with B_{Vax} or DCs. Five days after cellular adoptive transfer, tumor-bearing brains were collected and evaluated for the amount of proliferating CD8⁺ T cells, measured by the expression of Ki67. As shown in FIG. 4B, animals that received B_{Vax} displayed a higher amount of Ki67 CD8 CD45.1⁺ T cells.

### Combination Therapy Provides Long-Term Animal Survival

Activation of B cells can lead to the up-regulation of the immunoregulatory molecule PD-L1 (Freeman et al., 2000). This is a shared feature of B_{Vax}, as approximately 50% of the cells at the time of the animal injection express PD-L1 (FIG. 13A). This phenomenon could lead to adverse effects of consecutive B_{Vax} injections, as the acquisition of PD-L1 by B cells is associated with immunosuppressive functions in the context of cancers (Epeldegui et al., 2019; Guan et al., 2016; Lee-Chang et al., 2019a). Thus, the inventors hypothesized that adding anti-PD-L1 treatment could improve B_{Vax} effector function and therapeutic outcome. First, the inventors evaluated whether B_{Vax}+anti-PD-L1 improved CD8⁺ T-cell persistence in CT2A-bearing mice after RT. Naïve CD8⁺ T cells were fluorescently labeled with a lipophilic fluorescent dye (CellTracker). Seven days post-adoptive transfer, mice were evaluated for the Cell Tracker⁺ CD8⁺ T-cell abundancy. Tumor-bearing brains, dCLN and spleens showed significantly increased counts of adoptively transferred CD8⁺ T cells when B_{Vax} was administered together with anti-PD-L1 (FIG. 4C). Furthermore, immunophenotype analysis revealed that adoptively transferred CD8⁺ T cells acquired a CD44⁺CD62L^{Hi}IFNγ⁺ memory phenotype in vivo (FIG. 4D).

Next, CT2A-bearing mice received RT and three intravenous injections of B_{Vax} pulsed with CT2A lysates and CD8⁺ T cells. After each cell therapy, mice were given an intraperitoneal injection of anti-PD-L1 (500, 200 and 200 μg/mouse respectively). This combination provided a significant clinical benefit, with 80% of mice being long-term survivors (no RT median survival days: 18; Mock (RT): 25, anti-PD-L1: 28; CD8⁺ T +anti-PD-L1: 39, B_{Vax}+CD8⁺ T+anti-PD-L1: Undefined; anti-PD-L1 vs B_{Vax}+CD8⁺ T+anti-PD-L1 p<0.0001; FIG. 5A). Seventy-five days after tumor injection, surviving mice were re-challenged with CT2A in the opposite hemisphere (arrow, FIG. 5A). None of the mice developed any sign of tumor growth, and their clinical status was unchanged. After 245 days, surviving mice were sacrificed, and brains were evaluated for the presence of both tumor mass and CD8⁺ T cells. Brain sections of long-term survivors treated with RT, B_{Vax} and CD8⁺ T cells, and PD-L1 blockade (LTS-B_{Vax}+CD8) were obtained from the right hemisphere (first site of injection, LTS-CD8+B_{Vax} R) and left hemisphere (second site of injection, LTS-B_{Vax}+CD8 L). No sign of tumor mass, measured by hematoxylin and eosin (H&E) staining, was observed as compared to age-matched control CT2A-bearing brains sacrificed 14 days after tumor injection (FIG. 5B). A majority of CD8⁺ T cells in control tumor-bearing brains resided within the tumor vicinity, and minimal counts were found outside its boundaries (FIG. 13B). In contrast, in LTS-B_{Vax}+CD8 animals that lack tumor mass, CD8⁺ T cells were found nearby the injection site but also at different locations, such as the choroid plexus, the pons, and the cerebellum. This CD8⁺T-cell infiltration pattern was similar in both the right and left hemispheres (FIG. 5C). The further immuno-profiling analysis revealed that the majority of the infiltrating immune cells in the brains of both LTS-CD8 and LTS-B_{Vax}+CD8 mice were CD8⁺ T cells (FIG. 5D). Furthermore, brain-infiltrating CD8⁺ T cells from LTS-CD8+B_{Vax} mice showed an activated IFNγ -expressing phenotype. Importantly, there was a little-to-no expression of exhaustion and/or inhibitory molecules such as PD-1, KLGRI or TIGIT in all LTS groups when compared to CD8⁺ T cells from tumor-bearing brains of the controls (FIG. 5D). Only low amounts of myeloid cells or CD4⁺ T cells were observed. Within the CD4⁺ T-cell compartment, the expression of Foxp3 was drastically reduced. Only LTS-B_{Vax}+CD8 brains showed increased accumulation of 4-1BBL⁺IFNγ⁺ B cells (FIG. 13C), suggesting that B_{Vax} treatment might also promote the accumulation of proinflammatory B cells. Altogether, the data demonstrate that the combination of RT, B_{Vax}-based immunotherapy and checkpoint blockade successfully eradicated the tumor. This clinical observation was associated with infiltration and persistence of functional CD8⁺ T cells in the brain.

B_{Vax} Extend Animal Survival in Combination with GBM Standard-of-Care

Next, the inventors evaluated the effect of B_{Vax} treatment in combination with whole brain irradiation (total of 9Gy, fractionated in 3 times 3Gy, B-RT) and temozolomide (5 intraperitoneal injections of 50 mg/Kg, TMZ) in CT2A-bearing mice. Similar to whole body RT, serum BAFF levels were elevated in tumor-free and CT2A-bearing mice treated with TMZ, which was further increased when combined with B-RT (FIG. 6A). This suggests that standard of care could promote B-cell fitness in vivo. These results were associated with higher numbers of adoptively transferred CD45.1⁺ B_{Vax} in the spleen, dCLN and tumors of CT2A-bearing mice treated with standard of care therapy (FIG. 6B). B_{Vax} therapeutic effectiveness was first evaluated in mice that were treated with either B-RT (FIG. 6C) or TMZ (FIG. 6D). The inventors observed that only B_{Vax} in combination with CD8⁺ T cells significantly extend animal survival after B-RT treatment (Mock median survival: 20; B-RT: 26; B-RT+CD8⁺ T: 26; B-RT+B_{Vax}: 28, B-RT+B_{Vax} +CD8⁺ T: 30. B-RT vs B-RT+B_{Vax}+CD8⁺ T p=0.01; FIG. 6C). Similar results were obtained in tumor-bearing mice treated with TMZ (Mock median survival: 20; TMZ: 21; TMZ+CD8⁺ T: 21; TMZ+B_{Vax}: 21.5; TMZ+B_{Vax}+CD8⁺ T: 27.5. TMZ vs TMZ+B_{Vax}+CD8⁺ T p<0.0001; FIG. 6D). Next, the inventors tested the B_{Vax}+CD8⁺ T cellular therapy combination with both B-RT and TMZ. A group of mice also received anti-PD-L1, as the inventors observed previously that this checkpoint blockade strategy synergizes with B_{Vax} to eradicate the tumor (FIG. 5A). For this experiment, mice received 2 consecutive injections of B_{Vax} and CD8⁺ T cells. After each cell therapy, mice were given an intraperitoneal injection of anti-PD-L1. This combination provided a significant clinical benefit, with 50% of mice being long-term survivors (Mock median survival days: 20; B_{Vax}+CD8⁺ T: 26; B-RT+TMZ: 29; B-RT+TMZ+B_{Vax}+CD8⁺ T: 38.5; RT+TMZ +anti-PD-L1: 32; RT+TMZ +CD8⁺ T+anti-PD-L1: 33; RT+TMZ+B_{Vax}+CD8⁺ T+anti-PD-L1: 55; anti-PD-L1 vs B_{Vax}+CD8⁺ T+anti-PD-L1 p<0.0001; anti-PD-L1+CD8⁺ T vs B_{Vax} +CD8⁺ T+anti-PD-L1 p<0.0001; FIG. 6E). Altogether, this data demonstrates that combination of standard-of-care strategy (B-RT+TMZ), B_{Vax} -based cellular therapy and PD-L1 checkpoint blockade significantly enhanced animal survival.

GBM Patient-Derived B_{Vax} Expand and Activate Autologous Anti-Glioma CD8⁺ T Cells

Next, the inventors tested the ability of GBM patient-derived B_{Vax} to promote anti-tumor CD8⁺ T-cell response. 4-1BBL⁺ B cells and CD8⁺ T cells were isolated from GBM patient peripheral blood, followed by human B_{Vax} generation using the same protocol as murine B_{Vax}. The inventors then used freshly resected tumor as a source of protein homogenate (tumor lysate) to pulse B_{Vax} (FIG. 13D). The B_{Vax} (±tumor lysates) were tested for the ability to induce CD8⁺ T-cell activation by co-culturing pulsed B_{Vax} with autologous eFluor450-labeled CD8⁺ T cells. Cultures were supplemented with recombinant human IL2, and no exogenous TCR stimulators such as anti-CD3/CD28 were added to the culture. The inventors observed that CD8⁺ T cells cultured with B_{Vax} for 5 days expanded greatly and expressed high levels of GzmB. This observation was almost exclusive to B_{Vax} pulsed with autologous tumor lysates (expansion index mean %±SD: B_{Vax} VS. B_{Vax} (TUMOR LYSATE) 3.1±1.8 vs 6.05±1.4.p<0.05; % of GzmB expression in mean±SD: B_{Vax} VS. B_{Vax} (TUMOR LYSATE) 26.2±6.6 vs 49.3±18.55.p<0.05, FIG. 7A).

Lastly, the inventors tested the activated and expanded CD8⁺ T-cell's ability to kill tumor cells via an in vitro cytotoxicity assay. The results showed that CD8⁺ T cells activated via the pulsed-B_{Vax} system were able to potently kill glioma cells and spare non-tumor adherent cells (FIG. 13E), both in the context of newly diagnosed GBM (NU case 02120, FIG. 7B) and recurrence (NU case 02265, FIG. 7C). These results confirm the potency of B_{Vax} to promote CD8⁺ T-cell mediated anti-glioma immunity.

### B_{Vax} Produce Tumor-Reactive Immunoglobulins

After adoptive transfer, approximately half of B_{Vax} express CD138 (FIG. 8A), also known as syndecan-1, a molecule expressed in terminally differentiated Ab-producing cells (McCarron et al., 2017). Thus, next the inventors examined the humoral immune response of B_{Vax} and whether B_{Vax}-derived Abs participated in tumor clearance. To obtain B_{Vax}-derived immunoglobulins (Ig), B_{Vax} obtained from CT2A-bearing mice were intravenously injected into B-cell deficient (BKO) CT2A-bearing mice. Control groups receiving B_{Naive} and B_{Act} were also added to the experiment. Two weeks later, mice were sacrificed and serum were collected (FIG. 8B). Serum samples were used to measure Ig subtypes using ELISA. The inventors observed that B_{Naive} and B_{Act} predominantly produce IgM, while B_{Vax} mainly produced IgG1, IgG2a and IgG2b (FIG. 8C). In parallel, serum samples were tested for their reactivity to CT2A using immunofluorescence (IF) on CT2A-bearing brains from B-cell deficient mice. As these mice are deficient in mature B cells, they also lack of endogenous Ig production. Serum B_{Vax}-derived IgG reactivity was higher in the tumor area (T) when compared to healthy brain areas (B), but did not co-label with CD11b⁺ myeloid cells (FIG. 8D), suggesting that B_{Vax} produce tumor-specific Abs. To test whether these Abs can recognize tumor-associated Ags the inventors utilized the OVA-SIINFEKL (SEQ ID NO: 1) system. B_{Vax} first were generated from GL261 tumor-bearing mice expressing OVA (GL261-OVA) then were subsequently injected into GL261-OVA-bearing B-cell deficient mice. Two weeks after the cell adoptive transfer, serum IgG were purified using Protein A/G columns. After protein concentration normalization, samples were tested for their SIINFEKL (SEQ ID NO: 1) reactivity using ELISA. The inventors included Abs from SIINFEKL (SEQ ID NO: 1)-immunized mice as positive control. The inventors observed that SIINFEKL (SEQ ID NO: 1) reactivity was significantly increased in B_{Vax}-derived IgG when compared to B_{Naive}-derived IgG (FIG. 8E). Next, the inventors tested whether B_{Vax}-derived IgG (B_{Vax} IgG) could control tumor growth. B_{Vax} -derived IgG were collected as previously described and intracranially injected (3 consecutive times, 12.5 μg/injection). B_{Naive}-derived IgG (B_{Naive} IgG) were used as control. B_{Vax} IgG extended significantly animal survival (Mock median survival days: 20; B_{Naive} IgG: 16; B_{Vax} IgG: 31.5; Mock vs B_{Vax} IgG: p=0.017; FIG. 8F). These results confirm that humoral effector functions play a role in B_{Vax} anti-tumor properties.

### Discussion

In previous work, the inventors reported that in the CT2A glioma model, B-cell depletion using Rituximab has beneficial results only when B cells are depleted locally, sparing most of the peripheral B cells (Lee-Chang et al., 2019a). In contrast, systemic B-cell depletion does not reveal the same therapeutic effect. This highlights the possibility that B cells with distinct functions (anti- or pro-tumorigenesis) could be activated during tumor development. Accordingly, B-cell infiltration and formation of ectopic follicles within the tumor microenvironment have been recently associated with positive responsiveness to checkpoint blockade in melanoma and sarcomas (Cabrita et al., 2020; Helmink et al., 2020; Petitprez et al., 2020). However, GBM does not allow these lymphoid structures to be formed within the tumor microenvironment, as GBM restricts B-cell infiltration (Lee-Chang et al., 2019b) and is characterized by lymphodepletion (Thorsson et al., 2018). However, some B-cell subsets might still be able to promote an anti-tumor response. The 4-1BBL expression on B cells identifies Ag-experienced activated B cells (Futagawa et al., 2002). It was previously shown that 4-1BBL* B cells express high levels of proinflammatory cytokines such as TNFα, and co-stimulatory molecules such as CD86, which were shown to have a central role in CD8⁺ T-cell activation (Lee-Chang et al., 2016; Lee-Chang et al., 2014). In glioma-bearing mice, 4-1BBL⁺ B cells were found increased in the peripheral lymphoid organs and they differ from immunosuppressive B cells found in the tumor microenvironment (Lee-Chang et al., 2019a). In GBM patients' peripheral blood, the association between the levels of 4-1BBL expression by B cells, and the activation status of CD8⁺ T cells (expression of CD69), suggested a possible proinflammatory immune signature within these patients. Further functional studies ex-vivo confirmed that activation of CD8⁺ T cells was related to 4-1BBL⁺ B cells. Thus, the inventors considered this rare but highly activated B-cell subset as a potential cellular platform to boost CD8⁺ T-cell mediated tumor killing.

4-1BBL is the single known ligand for 4-1BB (Goodwin et al., 1993), a TNF receptor family co-stimulatory receptor that plays a fundamental role in activating Ag-experienced CD8⁺ T cells to establish long-term immunological memory (Melero et al., 1997; Uno et al., 2006). Thus, 4-1BB agonism continues to be an attractive strategy to boost CD8⁺ T-cell immunity in the context of different cancers, including non-Hodgkin lymphoma and melanoma (Chester et al., 2018). 4-1BBL expression in B cells require BCR and CD40 stimulation (Futagawa et al., 2002), and define a specific subset of activated B cells able to activate 4-1BB⁺ CD8⁺ T cells and promote anti-tumor immunity (Bodogai et al., 2015; Lee-Chang et al., 2016; Lee-Chang et al., 2014). Over-expression of 4-1BBL on the surface of APCs is transient and tightly controlled, as the aberrant presence of this marker might induce acute inflammation (Bukczynski et al., 2004; Vinay and Kwon, 1998). In this disclosure, the inventors utilized 4-1BBL⁺ activated B cells from glioma-bearing mice (secondary lymphoid organs) or GBM patient-derived PBMCs as a source of B-cell-based vaccine or, what the inventors term as B_{Vax}. In the aim to potentiate and stabilize the APC function, 4-1BBL⁺ B cells were further activated for a short time (48 hours) using CD40 and IFNγR activation and pulsed with tumor protein lysates to generate the vaccine.

Unlike naïve B cells, B_{Vax} were able to cross-present as potently as DCs in-vitro; thus, they could be considered as a professional APC. This agrees with a previous study that showed cross-presentation by B cells activates autoimmune CD8⁺ T cells in the context of type I diabetes (Marino et al., 2012). Most of B-cell-based vaccines utilize total circulating B cells (isolated using the CD20 or CD19 marker) and activated ex-vivo using CD40 agonism, Toll-like receptor ligands, homeostatic cytokines such as IL4 or IL21 (Wennhold et al., 2019). Some studies have used CD27⁺ memory B cells (Jourdan et al., 2017). However, in this disclosure the inventors show that sorting Ag-experienced B cells (via 4-1BBL), and endowing them with potent APC function, can be used as a unique tool in B-cell-based therapies. However, a limitation of this disclosure is the source of 4-1BBL⁺ B cells in the murine model and humans. The choice of using secondary lymphoid organs as a B_{Vax} source in the preclinical setting is because the volume of blood (and relative sparsity of 4-1BBL+ B-cells in circulation) makes PBMC-generated B_{Vax} from mice untenable.

In GBM patients, one consequence of the radio- and chemo-therapy is lymphopenia that can be profound and persistent (Grossman et al., 2015; Grossman et al., 2011; Nabors et al., 2011). A previous study showed that CD4⁺ T and CD19⁺ B cells are particularly affected by concomitant RT/TMZ administration (Ellsworth et al., 2014). Those authors observed that T-cell homeostatic factors such as IL7 or IL15 are unchanged, which suggests that in some patients, T cells are particularly vulnerable to standard-of-care (Ellsworth et al., 2014). In a parallel study, it was observed that in RT/TMZ-induced lymphopenic patients, levels of B-cell survival factor BAFF (also known as BLyS) were significantly elevated (Saraswathula et al., 2016), suggesting that the standard-of-care might provide a more optimal environment for B-cell adaption and persistence. This is clinically relevant as an important limitation of current cell-based immunotherapy is its lack of persistence in-vivo (DeRenzo and Gottschalk, 2019). Accordingly, RT treated CT2A-bearing mice showed sustained increased levels of BAFF in blood. BAFF is a universal homeostatic factor for all B-cell subtypes. This observation was associated with the ability of all B-cell subtypes tested (B_{Naive}, B_{Act} or B_{Vax}) to persist in-vivo. Treatment with BAFF receptor blocking Ab affected the survival of B_{Vax} in-vivo. Another interesting potential of B_{Vax} is the determination of their BCR repertoire and, by extension, their antibody specificity. How these antibodies may influence the anti-glioma immunity or tumor progression is a matter of ongoing study.

Tissue-recruitment of B_{Vax} is fundamental for their efficacy, as blockage of their migratory abilities toward secondary lymphoid organs abrogated their activating function of CD8⁺ T cells. The dependency between CD8⁺ T cells and B_{Vax} was strongly supported by the in-vivo tracking of CD8⁺ T cells, in which their accumulation in the tumor-bearing brains was enhanced when B_{Vax} was concomitantly administered. Optimal TCR stimulation by APC, and subsequent T-cell egress, occur 1-2 days after the interaction (Mempel et al., 2004). Accordingly, maximal accumulation of CD8⁺ T cells in the brain was observed 30 hours after cell B_{Vax} +CD8⁺ T-cell injection.

Like many immune cells, activated B cells express PD-L1, most likely as a mechanism to control inflammation. The inventors used this as a rationale to combine B_{Vax} and anti-PD-L1 immunotherapy. B_{Vax}+anti-PD-L1 treatment promoted CD8⁺ T-cell persistence in-vivo upon RT, in both tumor-bearing brains and draining-cervical lymph nodes. CD8⁺ T cells showed a remarkable memory phenotype and expression of IFNY, indicating an expansion of functional sentinel CD8⁺ T cells. Consistent with this, repeated administration of B_{Vax} and anti-PD-L1 allowed adoptively transferred CD8⁺ T cells to eradicate the tumor and prevent its regrowth upon reinjection in the opposite hemisphere in 80% of the treated mice. Tumor eradication correlated with prominent infiltration of CD8⁺ T cells in the injection sites (both in challenge and re-challenge sites). CD8⁺ T cells were also found in the choroid plexus, a structure known to play a fundamental role in central nervous system (CNS) immunosurveillance via the cerebrospinal fluid-brain-barrier (Wilson et al., 2010). However, CD8⁺ T cells were also present in more distant sites like the cerebellum and pons, suggesting organ-wide surveillance to protect the CNS. Accordingly, CNS infiltrating CD8⁺ T cells show an activated phenotype characterized by the expression of IFNγ and CD44, together with the absence of inhibitory molecules such as PD-1 or TIGIT. These findings suggest that fully functional memory-like CD8⁺ T cells persist in the target organ. Whether these cells arise from the adoptively transferred CD8⁺ T-cell pool, or newly differentiated cells upon lymphocyte replenishment due to the RT-driven lymphopenia, is a subject for future studies. Accumulation of activated, oligoclonal B cells were found in tumors of metastatic melanoma patients that responded to immune checkpoint blockade in neoadjuvant treatment settings (Helmink et al., 2020). A B cell lineage gene signature also correlated with responsiveness to PD1 blockade in sarcoma patients (Petitprez et al., 2020). Glioblastoma patients show poor B-cell infiltration, and those found in the tumor microenvironment present a strong immunosuppressive profile (Lee-Chang et al., 2019b). However, in a preclinical setting, LTS treated with both B_{Vax} and CD8⁺ T cells showed an accumulation of proinflammatory 4-1BBL⁺IFNγ⁺ B cells in the brain. It remains to be seen whether this plays a role in enhancing CD8⁺ T-cell response in the context of PD-L1 blockade. It is also important to note that repeated CD8⁺ T-cell adoptive transfer (without B_{Vax}) and anti-PD-L1 also eradicated the tumor in a small group of treated mice. These results suggest that a mechanism independent of B_{Vax} is also taking place to promote CD8⁺ T-cell-mediated CNS immunosurveillance. The inventors could hypothesize that anti-PD-L1 therapy could directly target tumor-associated myeloid cells (TAMCs), the most prominent non-neoplastic cell population in GBM (Zhang et al., 2019). Together with whole body RT, adoptively transferred CD8⁺ T cells could thrive within the tumor microenvironment and kill GBM cells.

Similar to whole body RT, B-RT+TMZ therapy promoted increased levels of serum BAFF, as seen in GBM patients that underwent standard-of-care (SoC) treatment (Sanchez-Perez et al., 2013; Saraswathula et al., 2016). Increased levels of serum BAFF correlated with enhanced B_{Vax} persistence in-vivo and with B_{Vax} therapeutic effect. Combination of SoC, B_{Vax} +CD8⁺ T-cells and PD-L1 blockade provided tumor eradication in 50% of treated mice. However, unlike mice treated with whole body RT, little to no effect was seen in CD8⁺ T-cell (without B_{Vax})+PD-L1 blockade in mice treated with whole brain RT. This suggests that GBM SoC provides a unique advantage for B-cell-based therapies over adoptive transfer therapies alone.

Finally, the inventors generated GBM patient-derived B_{Vax} that, after pulsing with protein lysates originated from the freshly resected tumor from the same patient, activated and expanded autologous CD8⁺ T cells. Those B_{Vax} -activated CD8⁺ T cells killed autologous glioma cells and, at the same time, spared adherent cells. The fact that no exogenous activation (ex: anti-CD3/CD28 activation) was required to induce CD8⁺ T-cell activation suggests that patient-derived B_{Vax} present tumor-associated Ags to Ag-experienced CD8⁺ T cells. In GBM patients, peripheral T cells share clonality with tumor-infiltrating lymphocytes (TILs) (Sims et al., 2016), suggesting that tumor-specific T cells might be present in the circulation and that B_{Vax} could be an ideal platform to expand these cells and boost their cytotoxic effect.

On major reason B_{Vax} outperform DC based on animal survival could be explained by the unique ability of activated B cells to eventually become Ab-producing cells in-vivo. These data show that B_{Vax} produce mainly IgGs that react to tumor cells and tumor-associated Ags, which extend animal survival. Thus, B_{Vax} might produce tumor-specific Abs able to cross the blood-brain-barrier and attack the tumor cells via Ab-dependent cell cytotoxicity. However, the inventors also observed that B_{Vax} can migrate and infiltrate the tumor. In addition, the results from long-term survivors shows that a substantial amount of 4-1BBL⁺IFNγ⁺ B cells are present in the brains 245 days tumor implantation. Thus, one could hypothesize that tumor-infiltrating B_{Vax} could seed an ectopic germinal center reaction and act as source of Abs in-situ. While further studies are needed to elucidate the exact reactivity of B_{Vax}-derived IgG and their effector immune functions, it is undeniable that B_{Vax} represents a unique immunotherapy platform that merge both cellular (CD8⁺ T-cell activation) and humoral (Ab production) function.

Overall, this disclosure proposes to utilize 4-1BBL⁺ B cells as a source of potent cellular and humoral immunotherapy. This is an autologous vaccine that only requires CD40 and IFNγR activation for a short time, which makes its clinical translation highly feasible.

### Example 2—Phenoypte of B_{Vax} In Vivo

### Materials and Methods

B_{Vax}, B_{Naive} and tumor-infiltrating B-cell (TI B) B-cell receptors (BCR) were analyzed by DNA sequencing of the immunoglobulin heavy chain (IgH) (FIG. 14). Significantly enriched clonotypes were analyzed using the Adaptive Biotech platform. Clonotypes were analyzed as amino acid sequences.

B_{Vax} and B_{Naive} were obtained from CD45.1 congenic mice. B cells were intravenously transferred into tumor-bearing and non-tumor-bearing mice B-cell deficient mice (CD45.2 genotype). CD45.1* B cells counts and phenotype were analyzed in the blood, brain, deep cervical lymph nodes (dCLN) and spleens after 72 hours by flow cytometry (FIG. 15).

### Results

Immunoglobulin heavy chain (IgH) DNA sequences were analyzed in B_{Vax} and compared to IgH sequences in naïve B cells (B_{Naive}) and tumor-infiltrating (TI) B cells (FIG. 14A). Three clones were found to be significantly enriched in B_{Vax} compared to B_{Naive} and TI B cells (gray, FIG. 14B). Six clones were significantly enriched in B_{Vax} compared to B_{Naive} and overlap with clones that are highly abundant in TI B cells (blue).

B_{Vax} obtained from CD45.1⁺ CT2A-bearing mice were injected intravenously into CD45.2⁺ CT2A-bearing mice (T) and naïve mice (NT). After 72 hours mice were sacrificed and analyzed for the presence of CD45.1⁺ cells in blood, tumor-bearing brains, deep cervical lymph nodes (dCLN) and the spleen. Only tumor-bearing brains harbored CD45.1⁺ B_{Vax} (FIG. 15A). Some CD45.1⁺ B_{Vax} infiltrating the tumor-bearing brains showed a plasmablast-like phenotype, as shown by the expression of plasmablast marker CD138 (FIG. 15B).

### Discussion

Sequencing of B cell receptors (BCRs) revealed that, as a population, B_{Vax} comprised 9 significantly enriched BCR clones when compared to B_{Naive}. Among these 9 clones, 6 clones overlapped with endogenous TI B cells, suggesting that within the heterogenous B_{Vax} pool of cells, a proportion of the clones are tumor reactive. This observation suggests that B_{Vax} may differentiate into plasmablasts intratumorally to locally produce antibodies specifically directed against the tumor.

### REFERENCES

Ahmadi, T., A. Flies, Y. Efebera, and D.H. Sherr. 2008. CD40 Ligand-activated, antigen-specific B cells are comparable to mature dendritic cells in presenting protein antigens and major histocompatibility complex class I- and class II-binding peptides. Immunology 124:129-140.
Bodogai, M., K. Moritoh, C. Lee-Chang, C.M. Hollander, C.A. Sherman-Baust, R.P. Wersto, Y. Araki, I. Miyoshi, L. Yang, G. Trinchieri, and A. Biragyn. 2015. Immunosuppressive and Prometastatic Functions of Myeloid-Derived Suppressive Cells Rely upon Education from Tumor-Associated B Cells. Cancer Res 75:3456-3465.
Braun, D., I. Caramalho, and J. Demengeot. 2002. IFN-alpha/beta enhances BCR-dependent B cell responses. Int Immunol 14:411-419.
Bruno, T.C., P.J. Ebner, B.L. Moore, O.G. Squalls, K.A. Waugh, E.B. Eruslanov, S. Singhal, J.D. Mitchell, W.A. Franklin, D.T. Merrick, M.D. McCarter, B.E. Palmer, J.A. Kern, and J.E. Slansky. 2017. Antigen-Presenting Intratumoral B Cells Affect CD4(+) TIL Phenotypes in Non-Small Cell Lung Cancer Patients. Cancer Immunol Res 5:898-907.
Bukczynski, J., T. Wen, K. Ellefsen, J. Gauldie, and T.H. Watts. 2004. Costimulatory ligand 4-1BBL (CD137L) as an efficient adjuvant for human antiviral cytotoxic T cell responses. Proc Natl Acad Sci U S A 101:1291-1296.
Cabrita, R., M. Lauss, A. Sanna, M. Donia, M. Skaarup Larsen, S. Mitra, I. Johansson, B. Phung, K. Harbst, J. Vallon-Christersson, A. van Schoiack, K. Lovgren, S. Warren, K. Jirstrom, H. Olsson, K. Pietras, C. Ingvar, K. Isaksson, D. Schadendorf, H. Schmidt, L. Bastholt, A. Carneiro, J.A. Wargo, I.M. Svane, and G. Jonsson. 2020. Tertiary lymphoid structures improve immunotherapy and survival in melanoma. Nature 577:561-565.
Chester, C., M.F. Sanmamed, J. Wang, and I. Melero. 2018. Immunotherapy targeting 4-1BB: mechanistic rationale, clinical results, and future strategies. Blood 131:49-57.
Cyster, J.G., and C.C. Goodnow. 1995. Pertussis toxin inhibits migration of B and T lymphocytes into splenic white pulp cords. J Exp Med 182:581-586.
DeRenzo, C., and S. Gottschalk. 2019. Genetic Modification Strategies to Enhance CAR T Cell Persistence for Patients With Solid Tumors. Front Immunol 10:218.
Eagles, M.E., F. Nassiri, J.H. Badhiwala, S. Suppiah, S.A. Almenawer, G. Zadeh, and K.D. Aldape. 2018. Dendritic cell vaccines for high-grade gliomas. Ther Clin Risk Manag 14:1299-1313.
Ellsworth, S., A. Balmanoukian, F. Kos, C.J. Nirschl, T.R. Nirschl, S.A. Grossman, L. Luznik, and C.G. Drake. 2014. Sustained CD4(+) T cell-driven lymphopenia without a compensatory IL-7/IL-15 response among high-grade glioma patients treated with radiation and temozolomide. Oncoimmunology 3:e27357.
Epeldegui, M., D.V. Conti, Y. Guo, W. Cozen, M.L. Penichet, and O. Martinez-Maza. 2019. Elevated numbers of PD-L1 expressing B cells are associated with the development of AIDS-NHL. Sci Rep 9:9371.
Freeman, G.J., A.J. Long, Y. Iwai, K. Bourque, T. Chernova, H. Nishimura, L.J. Fitz, N. Malenkovich, T. Okazaki, M.C. Byrne, H.F. Horton, L. Fouser, L. Carter, V. Ling, M.R. Bowman, B.M. Carreno, M. Collins, C.R. Wood, and T. Honjo. 2000. Engagement of the PD-1 immunoinhibitory receptor by a novel B7 family member leads to negative regulation of lymphocyte activation. J Exp Med 192: 1027-1034.
Fu, C., and A. Jiang. 2018. Dendritic Cells and CD8 T Cell Immunity in Tumor Microenvironment. Front Immunol 9:3059.
Futagawa, T., H. Akiba, T. Kodama, K. Takeda, Y. Hosoda, H. Yagita, and K. Okumura. 2002. Expression and function of 4-1BB and 4-1BB ligand on murine dendritic cells. Int Immunol 14:275-286.
Garaud, S., L. Buisseret, C. Solinas, C. Gu-Trantien, A. de Wind, G. Van den Eynden, C. Naveaux, J.N. Lodewyckx, A. Boisson, H. Duvillier, L. Craciun, L. Ameye, I. Veys, M. Paesmans, D. Larsimont, M. Piccart-Gebhart, and K. Willard-Gallo. 2019. Tumor infiltrating B-cells signal functional humoral immune responses in breast cancer. JCI Insight 5:
Gonzalez, N.K., K. Wennhold, S. Balkow, E. Kondo, B. Bolck, T. Weber, M. Garcia-Marquez, S. Grabbe, W. Bloch, M. von Bergwelt-Baildon, and A. Shimabukuro-Vornhagen. 2015. In vitro and in vivo imaging of initial B-T-cell interactions in the setting of B-cell based cancer immunotherapy. Oncoimmunology 4:e1038684.
Goodwin, R.G., W.S. Din, T. Davis-Smith, D.M. Anderson, S.D. Gimpel, T.A. Sato, C.R. Maliszewski, C.I. Brannan, N.G. Copeland, N.A. Jenkins, and et al. 1993. Molecular cloning of a ligand for the inducible T cell gene 4-1BB: a member of an emerging family of cytokines with homology to tumor necrosis factor. Eur J Immunol 23:2631-2641.
Grossman, S.A., S. Ellsworth, J. Campian, A.T. Wild, J.M. Herman, D. Laheru, M. Brock, A. Balmanoukian, and X. Ye. 2015. Survival in Patients With Severe Lymphopenia Following Treatment With Radiation and Chemotherapy for Newly Diagnosed Solid Tumors. J Natl Compr Canc Netw 13:1225-1231.
Grossman, S.A., X. Ye, G. Lesser, A. Sloan, H. Carraway, S. Desideri, S. Piantadosi, and N.C. Consortium. 2011. Immunosuppression in patients with high-grade gliomas treated with radiation and temozolomide. Clin Cancer Res 17:5473-5480.
Guan, H., Y. Wan, J. Lan, Q. Wang, Z. Wang, Y. Li, J. Zheng, X. Zhang, Z. Wang, Y. Shen, and F. Xie. 2016. PD-L1 is a critical mediator of regulatory B cells and T cells in invasive breast cancer. Sci Rep 6:35651.
Helmink, B.A., S.M. Reddy, J. Gao, S. Zhang, R. Basar, R. Thakur, K. Yizhak, M. Sade-Feldman, J. Blando, G. Han, V. Gopalakrishnan, Y. Xi, H. Zhao, R.N. Amaria, H.A. Tawbi, A.P. Cogdill, W. Liu, V.S. LeBleu, F.G. Kugeratski, S. Patel, M.A. Davies, P. Hwu, J.E. Lee, J.E. Gershenwald, A. Lucci, R. Arora, S. Woodman, E.Z. Keung, P.O. Gaudreau, A. Reuben, C.N. Spencer, E.M. Burton, L.E. Haydu, A.J. Lazar, R. Zapassodi, C.W. Hudgens, D.A. Ledesma, S. Ong, M. Bailey, S. Warren, D. Rao, O. Krijgsman, E.A. Rozeman, D. Peeper, C.U. Blank, T.N. Schumacher, L.H. Butterfield, M.A. Zelazowska, K.M. McBride, R. Kalluri, J. Allison, F. Petitprez, W.H. Fridman, C. Sautes-Fridman, N. Hacohen, K. Rezvani, P. Sharma, M.T. Tetzlaff, L. Wang, and J.A. Wargo. 2020. B cells and tertiary lymphoid structures promote immunotherapy response. Nature 577:549-555.
Iranzo, J., I. Martincorena, and E.V. Koonin. 2018. Cancer-mutation network and the number and specificity of driver mutations. Proc Natl Acad Sci U S A 115:E6010-E6019.
Jourdan, M., N. Robert, M. Cren, C. Thibaut, C. Duperray, A. Kassambara, M. Cogne, K. Tarte, B. Klein, and J. Moreaux. 2017. Characterization of human FCRL4-positive B cells. PLoS One 12:e0179793.
Kim, E.K., H.S. Seo, M.J. Chae, I.S. Jeon, B. Y. Song, Y.J. Park, H.M. Ahn, C.O. Yun, and C.Y. Kang. 2014. Enhanced antitumor immunotherapeutic effect of B-cell-based vaccine transduced with modified adenoviral vector containing type 35 fiber structures. Gene Ther 21:106-114.
Lapointe, R., A. Bellemare-Pelletier, F. Housseau, J. Thibodeau, and P. Hwu. 2003. CD40-stimulated B lymphocytes pulsed with tumor antigens are effective antigen-presenting cells that can generate specific T cells. Cancer Res 63:2836-2843.
Lee-Chang, C., M. Bodogai, K. Moritoh, X. Chen, R. Wersto, R. Sen, H.A. Young, M. Croft, L. Ferrucci, and A. Biragyn. 2016. Aging Converts Innate Bla Cells into Potent CD8+T Cell Inducers. J Immunol 196:3385-3397.
Lee-Chang, C., M. Bodogai, K. Moritoh, P.B. Olkhanud, A.C. Chan, M. Croft, J.A. Mattison, P.J. Holst, R.E. Gress, L. Ferrucci, F. Hakim, and A. Biragyn. 2014. Accumulation of 4-1BBL+B cells in the elderly induces the generation of granzyme-B+CD8+ T cells with potential antitumor activity. Blood 124:1450-1459.
Lee-Chang, C., A. Rashidi, J. Miska, P. Zhang, K.C. Pituch, D. Hou, T. Xiao, M. Fischietti, S.J. Kang, C.L. Appin, C. Horbinski, L.C. Platanias, A. Lopez-Rosas, Y. Han, I.V. Balyasnikova, and M.S. Lesniak. 2019a. Myeloid-Derived Suppressive Cells Promote B cell-Mediated Immunosuppression via Transfer of PD-L1 in Glioblastoma. Cancer Immunol Res
Li, Z., S. Bao, Q. Wu, H. Wang, C. Eyler, S. Sathornsumetee, Q. Shi, Y. Cao, J. Lathia, R.E. Mclendon, A.B. Hjelmeland, and J.N. Rich. 2009. Hypoxia-inducible factors regulate tumorigenic capacity of glioma stem cells. Cancer Cell 15:501-513.
Louveau, A., J. Herz, M.N. Alme, A.F. Salvador, M.Q. Dong, K.E. Viar, S.G. Herod, J. Knopp, J.C. Setliff, A.L. Lupi, S. Da Mesquita, E.L. Frost, A. Gaultier, T.H. Harris, R. Cao, S. Hu, J.R. Lukens, I. Smirnov, C.C. Overall, G. Oliver, and J. Kipnis. 2018. CNS lymphatic drainage and neuroinflammation are regulated by meningeal lymphatic vasculature. Nat Neurosci 21:1380-1391.
Mackay, F., and J.L. Browning. 2002. BAFF: a fundamental survival factor for B cells. Nat Rev Immunol 2:465-475.
Marino, E., B. Tan, L. Binge, C.R. Mackay, and S.T. Grey. 2012. B-cell cross-presentation of autologous antigen precipitates diabetes. Diabetes 61:2893-2905.
Maxwell, R., C.M. Jackson, and M. Lim. 2017. Clinical Trials Investigating Immune Checkpoint Blockade in Glioblastoma. Curr Treat Options Oncol 18:51.
McCarron, M.J., P.W. Park, and D.R. Fooksman. 2017. CD138 mediates selection of mature plasma cells by regulating their survival. Blood 129:2749-2759.
Melero, I., W.W. Shuford, S.A. Newby, A. Aruffo, J.A. Ledbetter, K.E. Hellstrom, R.S. Mittler, and L. Chen. 1997. Monoclonal antibodies against the 4-1BB T-cell activation molecule eradicate established tumors. Nat Med 3:682-685.
Mempel, T.R., S.E. Henrickson, and U.H. Von Andrian. 2004. T-cell priming by dendritic cells in lymph nodes occurs in three distinct phases. Nature 427:154-159.
Miska, J., A. Rashidi, A.L. Chang, M.E. Muroski, Y. Han, L. Zhang, and M.S. Lesniak. 2016. Anti-GITR therapy promotes immunity against malignant glioma in a murine model. Cancer Immunol Immunother 65:1555-1567.
Nabors, L.B., J.G. Supko, M. Rosenfeld, M. Chamberlain, S. Phuphanich, T. Batchelor, S. Desideri, X. Ye, J. Wright, S. Gujar, S.A. Grossman, and C.N.S.C. New Approaches to Brain Tumor Therapy. 2011. Phase I trial of sorafenib in patients with recurrent or progressive malignant glioma. Neuro Oncol 13:1324-1330.

Nielsen, J.S., R.A. Sahota, K. Milne, S.E. Kost, N.J. Nesslinger, P.H. Watson, and B.H. Nelson. 2012. CD20+ tumor-infiltrating lymphocytes have an atypical CD27-memory phenotype and together with CD8+ T cells promote favorable prognosis in ovarian cancer. Clin Cancer Res 18:3281-3292.
Petitprez, F., A. de Reynies, E.Z. Keung, T.W. Chen, C.M. Sun, J. Calderaro, Y.M. Jeng, L.P. Hsiao, L. Lacroix, A. Bougouin, M. Moreira, G. Lacroix, I. Natario, J. Adam, C. Lucchesi, Y.H. Laizet, M. Toulmonde, M.A. Burgess, V. Bolejack, D. Reinke, K.M. Wani, W.L. Wang, A.J. Lazar, C.L. Roland, J.A. Wargo, A. Italiano, C. Sautes-Fridman, H.A. Tawbi, and W.H. Fridman. 2020. B cells are associated with survival and immunotherapy response in sarcoma. Nature 577:556-560.
Pituch, K.C., J. Miska, G. Krenciute, W.K. Panek, G. Li, T. Rodriguez-Cruz, M. Wu, Y. Han, M.S. Lesniak, S. Gottschalk, and I. V. Balyasnikova. 2018. Adoptive Transfer of IL 13Ralpha2-Specific Chimeric Antigen Receptor T Cells Creates a Pro-inflammatory Environment in Glioblastoma. Mol Ther 26:986-995.
Prins, R.M., X. Wang, H. Soto, E. Young, D.N. Lisiero, B. Fong, R. Everson, W.H. Yong, A. Lai, G. Li, T.F. Cloughesy, and L.M. Liau. 2013. Comparison of glioma-associated antigen peptide-loaded versus autologous tumor lysate-loaded dendritic cell vaccination in malignant glioma patients. J Immunother 36:152-157.
Raychaudhuri, B., P. Rayman, J. Ireland, J. Ko, B. Rini, E.C. Borden, J. Garcia, M.A. Vogelbaum, and J. Finke. 2011. Myeloid-derived suppressor cell accumulation and function in patients with newly diagnosed glioblastoma. Neuro Oncol 13:591-599.
Sanchez-Perez, L., B.D. Choi, E.A. Reap, E.J. Sayour, P. Norberg, R.J. Schmittling, G.E. Archer, J.E. Herndon, 2nd, D.A. Mitchell, A.B. Heimberger, D.D. Bigner, and J.H. Sampson. 2013. BLyS levels correlate with vaccine-induced antibody titers in patients with glioblastoma lymphodepleted by therapeutic temozolomide. Cancer Immunol Immunother 62:983-987.
Saraswathula, A., E.A. Reap, B.D. Choi, R.J. Schmittling, P.K. Norberg, E.J. Sayour, J.E. Herndon, 2nd, P. Healy, K.L. Congdon, G.E. Archer, L. Sanchez-Perez, and J.H. Sampson. 2016. Serum elevation of B lymphocyte stimulator does not increase regulatory B cells in glioblastoma patients undergoing immunotherapy. Cancer Immunol Immunother 65:205-211.
Schultze, J.L., S. Michalak, M.J. Seamon, G. Dranoff, K. Jung, J. Daley, J.C. Delgado, J.G. Gribben, and L.M. Nadler. 1997. CD40-activated human B cells: an alternative source of highly efficient antigen presenting cells to generate autologous antigen-specific T cells for adoptive immunotherapy. J Clin Invest 100:2757-2765.
Sims, J.S., B. Grinshpun, Y. Feng, T.H. Ung, J.A. Neira, J.L. Samanamud, P. Canoll, Y. Shen, P.A. Sims, and J.N. Bruce. 2016. Diversity and divergence of the glioma-infiltrating T-cell receptor repertoire. Proc Natl Acad Sci U S A 113:E3529-3537.
Stupp, R., S. Taillibert, A. Kanner, W. Read, D. Steinberg, B. Lhermitte, S. Toms, A. Idbaih, M.S. Ahluwalia, K. Fink, F. Di Meco, F. Lieberman, J.J. Zhu, G. Stragliotto, D. Tran, S. Brem, A. Hottinger, E.D. Kirson, G. Lavy-Shahaf, U. Weinberg, C.Y. Kim, S.H. Paek, G. Nicholas, J. Bruna, H. Hirte, M. Weller, Y. Palti, M.E. Hegi, and Z. Ram. 2017. Effect of Tumor-Treating Fields Plus Maintenance Temozolomide vs Maintenance Temozolomide Alone on Survival in Patients With Glioblastoma: A Randomized Clinical Trial. JAMA 318:2306-2316.
Taphoorn, M.J.B., L. Dirven, A.A. Kanner, G. Lavy-Shahaf, U. Weinberg, S. Taillibert, S.A. Toms, J. Honnorat, T.C. Chen, J. Sroubek, C. David, A. Idbaih, J.C. Easaw, C.Y. Kim, J. Bruna, A.F. Hottinger, Y. Kew, P. Roth, R. Desai, J.L. Villano, E.D. Kirson, Z. Ram, and R. Stupp. 2018. Influence of Treatment With Tumor-Treating Fields on Health-Related Quality of Life of Patients With Newly Diagnosed Glioblastoma: A Secondary Analysis of a Randomized Clinical Trial. JAMA Oncol 4:495-504.
Thorsson, V., D.L. Gibbs, S.D. Brown, D. Wolf, D.S. Bortone, T.H. Ou Yang, E. Porta-Pardo, G.F. Gao, C.L. Plaisier, J.A. Eddy, E. Ziv, A.C. Culhane, E.O. Paull, I.K.A. Sivakumar, A.J. Gentles, R. Malhotra, F. Farshidfar, A. Colaprico, J.S. Parker, L.E. Mose, N.S. Vo, J. Liu, Y. Liu, J. Rader, V. Dhankani, S.M. Reynolds, R. Bowlby, A. Califano, A.D. Cherniack, D. Anastassiou, D. Bedognetti, Y. Mokrab, A.M. Newman, A. Rao, K. Chen, A. Krasnitz, H. Hu, T.M. Malta, H. Noushmehr, C.S. Pedamallu, S. Bullman, A.I. Ojesina, A. Lamb, W. Zhou, H. Shen, T.K. Choueiri, J.N. Weinstein, J. Guinney, J. Saltz, R.A. Holt, C.S. Rabkin, N. Cancer Genome Atlas Research, A.J. Lazar, J.S. Serody, E.G. Demicco, M.L. Disis, B.G. Vincent, and I. Shmulevich. 2018. The Immune Landscape of Cancer. Immunity 48:812-830 e814.
Tsou, P., H. Katayama, E.J. Ostrin, and S.M. Hanash. 2016. The Emerging Role of B Cells in Tumor Immunity. Cancer Res 76:5597-5601.
Uno, T., K. Takeda, Y. Kojima, H. Yoshizawa, H. Akiba, R.S. Mittler, F. Gejyo, K. Okumura, H. Yagita, and M.J. Smyth. 2006. Eradication of established tumors in mice by a combination antibody-based therapy. Nat Med 12:693-698.
Vega, E.A., M.W. Graner, and J.H. Sampson. 2008. Combating immunosuppression in glioma. Future Oncol 4:433-442.
Vinay, D.S., and B.S. Kwon. 1998. Role of 4-1BB in immune responses. Semin Immunol 10:481-489.
Wainwright, D.A., I.V. Balyasnikova, A.L. Chang, A.U. Ahmed, K.S. Moon, B. Auffinger, A.L. Tobias, Y. Han, and M.S. Lesniak. 2012a. IDO expression in brain tumors increases the recruitment of regulatory T cells and negatively impacts survival. Clin Cancer Res 18:6110-6121.
Wainwright, D.A., P. Nigam, B. Thaci, M. Dey, and M.S. Lesniak. 2012b. Recent developments on immunotherapy for brain cancer. Expert Opin Emerg Drugs 17:181-202.
Weller, M., N. Butowski, D.D. Tran, L.D. Recht, M. Lim, H. Hirte, L. Ashby, L. Mechtler, S.A. Goldlust, F. Iwamoto, J. Drappatz, D.M. O′Rourke, M. Wong, M.G. Hamilton, G. Finocchiaro, J. Perry, W. Wick, J. Green, Y. He, C.D. Turner, M.J. Yellin, T. Keler, T.A. Davis, R. Stupp, J.H. Sampson, and A.I.t. investigators. 2017. Rindopepimut with temozolomide for patients with newly diagnosed, EGFRvIII-expressing glioblastoma (ACT IV): a randomised, double-blind, international phase 3 trial. Lancet Oncol 18:1373-1385.
Wen, P.Y., D.A. Reardon, T.S. Armstrong, S. Phuphanich, R.D. Aiken, J.C. Landolfi, W.T. Curry, J.J. Zhu, M. Glantz, D.M. Peereboom, J.M. Markert, R. LaRocca, D.M. O′Rourke, K. Fink, L. Kim, M. Gruber, G.J. Lesser, E. Pan, S. Kesari, A. Muzikansky, C. Pinilla, R.G. Santos, and J.S. Yu. 2019. A Randomized Double-Blind Placebo-Controlled Phase II Trial of Dendritic Cell Vaccine ICT-107 in Newly Diagnosed Patients with Glioblastoma. Clin Cancer Res 25:5799-5807.
Wennhold, K., A. Shimabukuro-Vornhagen, and M. von Bergwelt-Baildon. 2019. B Cell-Based Cancer Immunotherapy. Transfus Med Hemother 46:36-46.
Wennhold, K., M. Thelen, H.A. Schlosser, N. Haustein, S. Reuter, M. Garcia-Marquez, A. Lechner, S. Kobold, F. Rataj, O. Utermohlen, G. Chakupurakal, S. Theurich, M. Hallek, H. Abken, A. Shimabukuro-Vornhagen, and M. von Bergwelt-Baildon. 2017. Using Antigen-Specific B Cells to Combine Antibody and T Cell-Based Cancer Immunotherapy. Cancer Immunol Res 5:730-743.
Wilson, E.H., W. Weninger, and C.A. Hunter. 2010. Trafficking of immune cells in the central nervous system. J Clin Invest 120:1368-1379.
Wintterle, S., B. Schreiner, M. Mitsdoerffer, D. Schneider, L. Chen, R. Meyermann, M. Weller, and H. Wiendl. 2003. Expression of the B7-related molecule B7-H1 by glioma cells: a potential mechanism of immune paralysis. Cancer Res 63:7462-7467.
Yoshizaki, A., T. Miyagaki, D.J. DiLillo, T. Matsushita, M. Horikawa, E.I. Kountikov, R. Spolski, J.C. Poe, W.J. Leonard, and T.F. Tedder. 2012. Regulatory B cells control T-cell autoimmunity through IL-21-dependent cognate interactions. Nature 491:264-268.
Zhang, P., J. Miska, C. Lee-Chang, A. Rashidi, W.K. Panek, S. An, M. Zannikou, A. Lopez-Rosas, Y. Han, T. Xiao, K.C. Pituch, D. Kanojia, I.V. Balyasnikova, and M.S. Lesniak. 2019. Therapeutic targeting of tumor-associated myeloid cells synergizes with radiation therapy for glioblastoma. Proc Natl Acad Sci U S A 116:23714-23723.

1. A method of making an anti-cancer composition comprising: (a) collecting 4-1BBL+ (CD137L+) B cells; (b) incubating the B cells with a CD40 agonist; (c) adding IFN-γ to the B cells; and (d) contacting the B cells with tumor-derived antigens.

2. The method of claim 1, wherein the B cells are collected from a subject diagnosed with cancer.

3. The method of claim 2, wherein the cancer is selected from the group consisting of glioblastoma, melanoma, breast cancer and pancreatic cancer.

4. The method of claim 1, wherein the CD40 agonist is selected from CD154 and a CD40 antibody or portion thereof capable of agonizing CD40.

5. The method of claim 1, wherein the B cells are also incubated with BAFF in step (b).

6. The method of claim 1, wherein the B cells are incubated with the CD40 agonist and optionally BAFF for at least 12 hours.

7. The method of claim 1, wherein the IFN-γ is added at a concentration of at least 10 U/ml.

8. The method of claim 1, wherein the IFN-γ is added for at least 12 hours prior to contacting with the tumor-derived antigen.

9. The method of claim 1, wherein the tumor-derived antigen is a tumor cell lysate.

10. (canceled)

11. A composition comprising 4-1BBL+ B cells made by the method of claim 1.

12. A composition comprising 4-1BBL+ B cells activated in vitro with a CD40 agonist and IFN-γ for at least 20 hours and pulsed with tumor-derived antigen.

13. The composition of claim 11, wherein the CD40 agonist is a CD40 antibody or portion thereof.

14. The composition of claim 12, wherein the tumor-derived antigen is a tumor cell lysate.

15. A method of using the B cell composition of claim 12 to treat a subject with cancer, the method comprising administering an effective amount of the composition to the subject.

16. The method of claim 15, further comprising administering radiation therapy to the subject.

17. (canceled)

18. The method of claim 15, further comprising administering a checkpoint inhibitor.

19. The method of claim 18, wherein the checkpoint inhibitor is an inhibitor of PD1 or PD-L1, optionally wherein the inhibitor is a PD1 or PD-L1 antibody or portion thereof.

20. The method of claim 15, further comprising administering additional non-B cell lymphocytes to the subject.

21. (canceled)

22. The method of claim 15, further comprising administering a chemotherapeutic to the subject.

23. (canceled)

24. The method of claim 15, wherein the composition is administered intravenously, sub-cutaneous, intra-lymphatic, intra-dermic or intracranially.
